(19) United States
(12) Patent Application Publication
(10) Pub. No.: US 2024/0207398 A1
(43) Pub. Date: Jun. 27, 2024

(54) METHODS AND COMPOSITIONS FOR TREATING CANCER

(71) Applicants: Genentech, Inc., South San Francisco, CA (US); Hoffmann-La Roche Inc., Little Falls, NJ (US)

(72) Inventors: Qingyuan LIU, Shanghai (CN); Anila TAHIRI, Basel (CH); Zhao ZHANG, Shanghai (CN); Edward Namserk CHA, San Carlos, CA (US)

(21) Appl. No.: 18/423,520

(22) Filed: Jan. 26, 2024

Related U.S. Application Data
(63) Continuation of application No. PCT/US2022/074278, filed on Jul. 28, 2022.

(30) Foreign Application Priority Data
Jul. 28, 2021 (WO) PCT/CN2021/109058

(51) Int. Cl. A61K 39/395; A61K 31/282; A61K 31/513; A61K 31/7068; A61K 38/17

(52) U.S. Cl. CPC A61K 39/3955; A61K 31/282; A61K 31/513; A61K 31/7068; A61K 38/1774

(57) ABSTRACT

The invention provides methods and compositions for use in treating cancer, e.g., gastric cancer (e.g., a gastric carcinoma (GC) or a gastroesophageal junction carcinoma (GEJC) (e.g., inoperable, locally advanced, metastatic, or advanced GC or GEJC)) or rectal cancer (e.g., locally advanced rectal cancer (LARC)) in a subject, for example, by administering to the subject a treatment regimen that includes an anti-TIGIT antagonist antibody (e.g., tiragolumab) in combination with a PD-1 axis binding antagonist (e.g., atezolizumab). The treatment regimen may be administered with chemotherapy or following a neoadjuvant chemotherapy regimen. Also provided are compositions (e.g., compositions comprising a PD-1 axis binding antagonist (e.g., atezolizumab) and/or an anti-TIGIT antagonist antibody (e.g., tiragolumab), including pharmaceutical compositions thereof, kits thereof, and articles of manufacture thereof) for use in treating cancer, e.g., gastric cancer (e.g., a GC or a GEJC (e.g., inoperable, locally advanced, metastatic, or advanced GC or GEJC)) or rectal cancer (e.g., LARC) in a subject.

### SEQUENCE LISTING

The instant application contains a Sequence Listing which has been submitted electronically in XML format and is hereby incorporated by reference in its entirety. Said XML copy, created on Jan. 8, 2024, is named 50474-260003_Sequence_Listing_1_8_24 and is 33,409 bytes in size.

### FIELD OF THE INVENTION

This invention relates to methods and compositions for use in treating cancer, e.g., gastric cancer (e.g., a gastric carcinoma (GC) or a gastroesophageal junction carcinoma (GEJC) (e.g., inoperable, locally advanced, metastatic, or advanced GC or GEJC)) or rectal cancer (e.g., locally advanced rectal cancer (LARC)) in a subject, for example, by administering to the subject a treatment regimen that includes an anti-T-cell immunoreceptor with Ig and ITIM domains (TIGIT) antagonist antibody (e.g., tiragolumab) and a PD-1 axis binding antagonist (e.g., atezolizumab). The treatment regimen may be administered with chemotherapy or following a neoadjuvant chemotherapy regimen.

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

Gastric carcinoma (GC) is the fifth leading cancer and the fourth leading cause of cancer-related deaths globally. In China, GC remains the second in terms of incidence among all malignancies and ranks third among cancers with respect to mortality. In China, more than 80% of patients with GC are already in advanced stages of the disease at the time of diagnosis. New treatment options are needed to improve survival and response as well as decrease toxicity in the first-line treatment setting of GC and gastroesophageal junction carcinoma (GEJC).

Colorectal cancer (CRC) remains a major cause of cancer deaths worldwide, ranking third in terms of incidence and second in terms of mortality. In China, it is the fifth leading cause of cancer deaths among both men and women. Rectal cancer is a malignancy originating from the rectum, accounting for approximately 40% of all CRC cases. The prognosis for patients with metastatic CRC remains poor, with a median 5-year survival of only 12.5%. There is a concerning rise in patients presenting with locally advanced disease, especially rectal cancer. For locally advanced rectal cancer, the pathological complete response rate of capecitabine- or 5-FU-based chemoradiotherapy is approximately 14%.

Thus, there is an unmet need in the field for the development of efficacious immunotherapies for the treatment of gastric cancer and rectal cancer.

### SUMMARY OF THE INVENTION

In one aspect, the invention provides a method for treating a subject having a gastric carcinoma (GC) or a gastroesophageal junction carcinoma (GEJC), the method comprising administering to the subject one or more dosing cycles of an anti-TIGIT antagonist antibody, a PD-1 axis binding antagonist, capecitabine, and oxaliplatin.

In some aspects, the GC or GEJC is an inoperable, locally advanced, metastatic, or advanced GC or GEJC.

In some aspects, the GC or GEJC is human epidermal growth factor receptor 2 (HER2)-negative.

In some aspects, the GC or GEJC is an adenocarcinoma.

In some aspects, the subject has not received a prior systemic therapy for GC or GEJC.

In some aspects, the method comprises administering to the subject: (a) the anti-TIGIT antagonist antibody at a fixed dose of about 600 mg every three weeks; (b) the PD-1 axis binding antagonist at a fixed dose of about 1200 mg every three weeks; (c) capecitabine at a dose of 1000 mg/m² twice daily for two weeks; and (d) oxaliplatin at a dose of 130 mg/m² every three weeks.

In some aspects, the length of each of the one or more dosing cycles is 21 days.

In some aspects, the method comprises administering to the subject the anti-TIGIT antagonist antibody, the PD-1 axis binding antagonist, and oxaliplatin on about Day 1 of each of the one or more dosing cycles.

In some aspects, the method comprises administering to the subject capecitabine on Days 1-14 of each of the one or more dosing cycles.

In some aspects, the method comprises administering to the subject the PD-1 axis binding antagonist before the anti-TIGIT antagonist antibody.

In some aspects, the method comprises administering to the subject the anti-TIGIT antagonist antibody, the PD-1 axis binding antagonist, and the oxaliplatin intravenously.

In some aspects, the method comprises administering to the subject the capecitabine orally.

In some aspects, the treating results in an increase in objective response rate (ORR) as compared to a reference ORR. In some aspects, the reference ORR is an ORR of a population of subjects who have received: (a) a treatment comprising capecitabine and oxaliplatin and not comprising a PD-1 axis binding antagonist and an anti-TIGIT antagonist antibody; and/or (b) a treatment comprising capecitabine, oxaliplatin, and a PD-1 axis binding antagonist and not comprising an anti-TIGIT antagonist antibody.

In some aspects, the treating results in an increase in progression-free survival (PFS) as compared to a reference PFS. In some aspects, the reference PFS is a median PFS of a population of subjects who have received: (a) a treatment comprising capecitabine and oxaliplatin and not comprising a PD-1 axis binding antagonist and an anti-TIGIT antagonist antibody; and/or (b) a treatment comprising capecitabine, oxaliplatin, and a PD-1 axis binding antagonist and not comprising an anti-TIGIT antagonist antibody.

In some aspects, the treating results in an increase in overall survival (OS) as compared to a reference OS. In some aspects, the reference OS is a median OS of a population of subjects who have received: (a) a treatment comprising capecitabine and oxaliplatin and not comprising a PD-1 axis binding antagonist and an anti-TIGIT antagonist antibody; and/or (b) a treatment comprising capecitabine, oxaliplatin, and a PD-1 axis binding antagonist and not comprising an anti-TIGIT antagonist antibody.

In some aspects, the treating results in an increase in duration of response (DOR) as compared to a reference DOR. In some aspects, the reference DOR is a median DOR of a population of subjects who have received: (a) a treatment comprising capecitabine and oxaliplatin and not comprising a PD-1 axis binding antagonist and an anti-TIGIT antagonist antibody; and/or (b) a treatment comprising capecitabine, oxaliplatin, and a PD-1 axis binding antagonist and not comprising an anti-TIGIT antagonist antibody.

In another aspect, the invention provides a method for treating a subject having a rectal cancer, the method comprising administering to the subject one or more dosing cycles of an anti-TIGIT antagonist antibody and a PD-1 axis binding antagonist, wherein the one or more dosing cycles are performed following a neoadjuvant chemotherapy (nCRT) regimen.

In some aspects, the rectal cancer is a stage cT₃N+M₀ or stage cT₄N_{any}M₀ rectal cancer.

In some aspects, the rectal cancer is an adenocarcinoma.

In some aspects, the subject does not have synchronous colon cancer.

In some aspects, the subject has not received a prior therapy for rectal cancer.

In some aspects, the method comprises administering to the subject the anti-TIGIT antagonist antibody at a fixed dose of about 600 mg every three weeks and the PD-1 axis binding antagonist at a fixed dose of about 1200 mg every three weeks.

In some aspects, the length of each of the one or more dosing cycles is 21 days.

In some aspects, the method comprises administering to the subject the anti-TIGIT antagonist antibody and the PD-1 axis binding antagonist on about Day 1 of each of the one or more dosing cycles.

In some aspects, the method comprises administering to the subject the PD-1 axis binding antagonist before the anti-TIGIT antagonist antibody.

In some aspects, the method comprises administering to the subject the anti-TIGIT antagonist antibody and PD-1 axis binding antagonist intravenously.

In some aspects, the one or more dosing cycles are initiated about two weeks after the last cycle of nCRT.

In some aspects, the one or more dosing cycles are initiated within four weeks after the last cycle of nCRT.

In some aspects, the nCRT regimen comprises radiotherapy delivered to the pelvis at a fraction of about 1.8 Gy per treatment. In some aspects, the radiotherapy is administered on Days 1-5 every week.

In some aspects, the nCRT regimen comprises administering a total of between about 45 and about 50.4 Gy of the radiotherapy to the subject.

In some aspects, the radiotherapy is administered in 25 to 28 fractions.

In some aspects, the nCRT regimen comprises a fluoropyrimidine-based chemotherapy.

In some aspects, the fluoropyrimidine-based chemotherapy is capecitabine or 5-fluorouracil (5-FU).

In some aspects, the capecitabine is administered orally at a dose of about 825 mg/m².

In some aspects, the capecitabine is administered orally twice daily on five consecutive days every week.

In some aspects, the capecitabine is administered orally twice daily on seven consecutive days every week.

In some aspects, the 5-FU is administered intravenously at a dose of about 225 mg/m².

In some aspects, the 5-FU is administered on five consecutive days every week.

In some aspects, the 5-FU is administered on seven consecutive days every week.

In some aspects, the nCRT is performed for 5 cycles.

In some aspects, the first dosing cycle of the anti-TIGIT antagonist antibody and PD-1 axis binding antagonist is initiated prior to a surgery.

In some aspects, three dosing cycles are completed prior to the surgery.

In some aspects, the surgery is performed within about four weeks after the last dosing cycle.

In some aspects, the surgery is radical surgical resection using total mesorectal excision (TME) and lymph node dissection.

In some aspects, the treating results in a pathological complete response (pCR) and/or an increase in pCR rate as compared to a reference pCR rate. In some aspects, the reference pCR rate is a pCR rate of population of subjects who have received a treatment comprising: (a) nCRT not followed by treatment with a PD-1 axis binding antagonist and an anti-TIGIT antagonist antibody; and/or (b) nCRT followed by treatment with a PD-1 axis binding antagonist.

In some aspects, the treating results in an increase in R0 resection rate as compared to a reference R0 resection rate. In some aspects, the reference R0 resection rate is an R0 resection rate of a population of subjects who have received a treatment comprising: (a) nCRT not followed by treatment with a PD-1 axis binding antagonist and an anti-TIGIT antagonist antibody; and/or (b) nCRT followed by treatment with a PD-1 axis binding antagonist.

In some aspects, the treating results in an increase in objective response rate (ORR) as compared to a reference ORR. In some aspects, the reference ORR is an ORR of a population of subjects who have received a treatment comprising: (a) nCRT not followed by treatment with a PD-1 axis binding antagonist and an anti-TIGIT antagonist antibody; and/or (b) nCRT followed by treatment with a PD-1 axis binding antagonist.

In some aspects, the treating results in an increase in relapse-free survival (RFS) rate as compared to a reference RFS rate. In some aspects, the reference RFS rate is an RFS rate of a population of subjects who have received a treatment comprising: (a) nCRT not followed by treatment with a PD-1 axis binding antagonist and an anti-TIGIT antagonist antibody; and/or (b) nCRT followed by treatment with a PD-1 axis binding antagonist. In some aspects, the RFS rate is a one-year RFS rate, a two-year RFS rate, or a three-year RFS rate.

In some aspects, the treating results in an increase in event-free survival (EFS) rate as compared to a reference EFS rate. In some aspects, the reference EFS rate is an EFS rate of a population of subjects who have received a treatment comprising: (a) nCRT not followed by treatment with a PD-1 axis binding antagonist and an anti-TIGIT antagonist antibody; and/or (b) nCRT followed by treatment with a PD-1 axis binding antagonist. In some aspects, the EFS rate is a one-year RFS rate, a two-year EFS rate, or a three-year EFS rate.

In some aspects, the anti-TIGIT antagonist antibody comprises the following hypervariable regions (HVRs): an HVR-H1 sequence comprising the amino acid sequence of SNSAAWN (SEQ ID NO: 11); an HVR-H2 sequence comprising the amino acid sequence of KTYYRFKWYSDYAVSVKG (SEQ ID NO: 12); an HVR-H3 sequence comprising the amino acid sequence of ESTTYDLLAGPFDY (SEQ ID NO: 13); an HVR-L1 sequence comprising the amino acid sequence of KSSQTVLYSSNNKKYLA (SEQ ID NO: 14); an HVR-L2 sequence comprising the amino acid sequence of WASTRES (SEQ ID NO: 15); and an HVR-L3 sequence comprising the amino acid sequence of QQYYSTPFT (SEQ ID NO: 16).

In some aspects, the anti-TIGIT antagonist antibody further comprises the following light chain variable region framework regions (FRs): an FR-L1 comprising the amino acid sequence of DIVMTQSPDSLAVSLGERATINC (SEQ ID NO: 17); an FR-L2 comprising the amino acid sequence of WYQQKPGOPPNLLIY (SEQ ID NO: 18); an FR-L3 comprising the amino acid sequence of GVPDRFSGSGSGTDFTLTISSLQAEDVAVYYC (SEQ ID NO: 19); and an FR-L4 comprising the amino acid sequence of FGPGTKVEIK (SEQ ID NO: 20).

In some aspects, the anti-TIGIT antagonist antibody further comprises the following heavy chain variable region FRs: an FR-H1 comprising the amino acid sequence of X₁VQLQQSGPGLVKPSQTLSLTCAISGDSVS (SEQ ID NO: 21), wherein X₁ is E or Q; an FR-H2 comprising the amino acid sequence of WIRQSPSRGLEWLG (SEQ ID NO: 22); an FR-H3 comprising the amino acid sequence of RITINPDTSKNQFSLOLNSVTPEDTAVFYCTR (SEQ ID NO: 23); and an FR-H4 comprising the amino acid sequence of WGQGTLVTVSS (SEQ ID NO: 24). In some aspects, X₁ is E. In some aspects, X₁ is Q.

In some aspects, the anti-TIGIT antagonist antibody comprises: (a) a heavy chain variable (VH) domain comprising an amino acid sequence having at least 95% sequence identity to the amino acid sequence of SEQ ID NO: 27 or 28; (b) a light chain variable (VL) domain comprising an amino acid sequence having at least 95% sequence identity to the amino acid sequence of SEQ ID NO: 29; or (c) a VH domain as in (a) and a VL domain as in (b).

In some aspects, the anti-TIGIT antagonist antibody comprises: (a) a VH domain comprising the amino acid sequence of SEQ ID NO: 27 and a VL domain comprising the amino acid sequence of SEQ ID NO: 29; or (b) VH domain comprising the amino acid sequence of SEQ ID NO: 28 and a VL domain comprising the amino acid sequence of SEQ ID NO: 29.

In some aspects, the anti-TIGIT antagonist antibody is a monoclonal antibody.

In some aspects, the anti-TIGIT antagonist antibody is a human antibody.

In some aspects, the anti-TIGIT antagonist antibody is a full-length antibody.

In some aspects, the anti-TIGIT antagonist antibody is tiragolumab.

In some aspects, the anti-TIGIT antagonist antibody is an antibody fragment that binds TIGIT selected from the group consisting of Fab, Fab′, Fab′-SH, Fv, single chain variable fragment (scFv), and (Fab′)2 fragments.

In some aspects, the anti-TIGIT antagonist antibody is an IgG class antibody.

In some aspects, the IgG class antibody is an IgG1 subclass antibody.

In some aspects, the anti-TIGIT antagonist antibody is tiragolumab, vibostolimab, etigilimab, EOS084448, SGN-TGT, TJ-T6, BGB-A1217, AB308, domvanalimab, BMS-986207, ASP8374, or COM902.

In some aspects, the method comprises administering to the subject the PD-1 axis binding antagonist at a fixed dose of about 1200 mg every three weeks.

In some aspects, the PD-1 axis binding antagonist is selected from the group consisting of a PD-L1 binding antagonist, a PD-1 binding antagonist, and a PD-L2 binding antagonist.

In some aspects, the PD-1 axis binding antagonist is a PD-L1 binding antagonist.

In some aspects, the PD-L1 binding antagonist inhibits the binding of PD-L1 to one or more of its ligand binding partners.

In some aspects, the PD-L1 binding antagonist inhibits the binding of PD-L1 to PD-1, B7-1, or both PD-1 and B7-1.

In some aspects, the PD-L1 binding antagonist is an anti-PD-L1 antagonist antibody.

In some aspects, the anti-PD-L1 antagonist antibody is atezolizumab, MDX-1105, durvalumab, avelumab, SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636.

In some aspects, the anti-PD-L1 antagonist antibody is atezolizumab.

In some aspects, the anti-PD-L1 antagonist antibody comprises the following HVRs: an HVR-H1 sequence comprising the amino acid sequence of GFTFSDSWIH (SEQ ID NO: 3); an HVR-H2 sequence comprising the amino acid sequence of AWISPYGGSTYYADSVKG (SEQ ID NO: 4); an HVR-H3 sequence comprising the amino acid sequence of RHWPGGFDY (SEQ ID NO: 5); an HVR-L1 sequence comprising the amino acid sequence of RASQDVSTAVA (SEQ ID NO: 6); an HVR-L2 sequence comprising the amino acid sequence of SASFLYS (SEQ ID NO: 7); and an HVR-L3 sequence comprising the amino acid sequence of QQYLYHPAT (SEQ ID NO: 8).

In some aspects, the anti-PD-L1 antagonist antibody comprises: (a) a heavy chain variable (VH) domain comprising an amino acid sequence having at least 95% sequence identity to the amino acid sequence of SEQ ID NO: 9; (b) a light chain variable (VL) domain comprising an amino acid sequence having at least 95% sequence identity to the amino acid sequence of SEQ ID NO: 10; or (c) a VH domain as in (a) and a VL domain as in (b).

In some aspects, the anti-PD-L1 antagonist antibody comprises: (a) a VH domain comprising the amino acid sequence of SEQ ID NO: 9; and (b) a VL domain comprising the amino acid sequence of SEQ ID NO: 10.

In some aspects, the anti-PD-L1 antagonist antibody is a monoclonal antibody.

In some aspects, the anti-PD-L1 antagonist antibody is a humanized antibody.

In some aspects, the anti-PD-L1 antagonist antibody is a full-length antibody.

In some aspects, the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

In some aspects, the anti-PD-L1 antagonist antibody is an IgG class antibody.

In some aspects, the IgG class antibody is an IgG1 subclass antibody.

In some aspects, the PD-1 axis binding antagonist is a PD-1 binding antagonist.

In some aspects, the PD-1 binding antagonist inhibits the binding of PD-1 to one or more of its ligand binding partners.

In some aspects, the PD-1 binding antagonist inhibits the binding of PD-1 to PD-L1, PD-L2, or both PD-L1 and PD-L2.

In some aspects, the PD-1 binding antagonist is an anti-PD-1 antagonist antibody.

In some aspects, the anti-PD-1 antagonist antibody is nivolumab, pembrolizumab, MEDI-0680, spartalizumab, cemiplimab, BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, or hAb21.

In some aspects, the PD-1 binding antagonist is an Fc fusion protein.

In some aspects, the Fc fusion protein is AMP-224.

In some aspects, the subject is a human.

### BRIEF DESCRIPTION OF THE DRAWINGS

FIG. 1 is a flow chart showing the study design of the YO43408 Phase Ib/II clinical trial, which enrolls patients with gastric or gastroesophageal junction carcinoma. Atezo=atezolizumab; CAPOX=capecitabine plus oxaliplatin; GC=gastric carcinoma; GEJC=gastroesophageal junction carcinoma; R=randomization; Tira=tiragolumab.

FIG. 2 is a schematic diagram showing an overview of the study schedule and activities in the safety run-in phase and randomization phase (Arm A and Arm B) of the ML43050 Phase II clinical trial, which enrolls patients with locally advanced rectal cancer. Timing of the collection of tumor and blood samples (row titled “Sample required”) is shown. Timing of the administration of chemoradiotherapy (dark gray block), tiragolumab (black triangle), and atezolizumab (light gray triangle), as well as surgery (white triangle) and pathological response evaluation (dark gray triangle) are indicated at various time points. RC=rectal cancer; LARC=locally advanced rectal cancer.

### DETAILED DESCRIPTION OF THE INVENTION

The present invention provides therapeutic methods and compositions for treatment of cancer, for example, gastric cancer (e.g., gastric carcinoma (GC) or gastroesophageal junction carcinoma (GEJC), e.g., inoperable, locally advanced, metastatic, or advanced GC or GEJC) or rectal cancer (e.g., locally advanced rectal cancer (LARC)). The invention is based, at least in part, on the discovery that immunotherapies including an anti-TIGIT antibody (e.g., an anti-TIGIT antagonist antibody, such as tiragolumab) in combination with a PD-1 axis binding antagonist (e.g., an anti-programmed death ligand-1 (PD-L1) antibody (e.g., atezolizumab) or an anti-programmed death-1 (PD-1) antibody) can be useful in the treatment of cancer. In certain aspects, the invention features combinations of an anti-TIGIT antibody (e.g., tiragolumab), a PD-1 axis binding antagonist (e.g., atezolizumab), and one or more chemotherapeutic agents (e.g., a platinum agent (e.g., oxaliplatin) and/or one or more fluoropyrimidine-based chemotherapy agents (e.g., capecitabine or 5-fluorouracil (5-FU))). In certain aspects, the invention features an anti-TIGIT antibody and a PD-1 axis binding antagonist (e.g., atezolizumab) administered following a neoadjuvant chemoradiotherapy (nCRT) regimen. Compositions, uses, and kits involving such combinations and/or dosing regimens are also provided herein.

### I. Definitions

The following abbreviations are used herein:

| | |
|---|---|
| CAS | Chemical Abstracts Service |
| CDR | complementarity determining region |
| CR | complete response |
| DNA | deoxyribonucleic acid |
| DOR | duration of response |
| Fab | fragment antigen-binding |
| Fc | fragment crystallizable |
| FFPE | formalin-fixed and paraffin-embedded |
| FR | framework |
| GC | gastric carcinoma |
| GEJC | gastroesophageal junction carcinoma |
| HVR | hypervariable region |
| IHC | immunohistochemistry |
| nCRT | neoadjuvant chemoradiotherapy |
| ORR | overall response rate/objective response rate |
| OS | overall survival |
| PD-1 | programmed death 1 |
| PD-L1 | programmed death ligand 1 |
| PD-L2 | programmed death ligand 2 |
| PFS | progression-free survival |
| PR | partial response |
| RNA | ribonucleic acid |

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

As used herein, “achieving a clinical response” refers to achieving one or more indicators of therapeutic efficacy for a disease (e.g., a cancer, e.g., a gastric cancer, e.g., gastric carcinoma (GC) or a gastroesophageal junction carcinoma (GEJC) (e.g., inoperable, locally advanced, metastatic, or advanced GC or GEJC), or rectal cancer (e.g., locally advanced rectal cancer (LARC))) in a subject or population of subjects during or following treatment with one or more agents intended to treat the disease (e.g., during or following a dosing regimen comprising one or more agents, e.g., during or following a dosing regimen comprising one or more dosing cycles of tiragolumab and atezolizumab), wherein the improvement is attributed to the treatment. The indicator of therapeutic efficacy may be, e.g., progression-free survival (PFS) (e.g., an increase in PFS as compared to a reference PFS); overall survival (OS) (e.g., an increase in OS as compared to a reference OS); a partial response (PR); a complete response (CR); a pathological complete response (pCR); an increased in the R0 resection rate as compared to a reference R0 resection rate; an increase in event-free survival (EFS) rate as compared to a reference EFS rate; an increase in relapse-free survival (RFS) rate as compared to a reference RFS rate; a reduction in the sum of longest diameters (SLD) of one or more target lesions; an increase in objective response rate (ORR) as compared to a reference ORR; or an increase in duration of response (DOR) as compared to a reference DOR which is a median DOR of a population of subjects.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

As used herein, “tiragolumab” is a fully human IgG1/kappa MAb-derived in Open Monoclonal Technology (OMT) rats that binds TIGIT and comprises the heavy chain sequence of SEQ ID NO: 33 and the light chain sequence of SEQ ID NO: 34. Tiragolumab comprises two N-linked glycosylation sites (N306) in the Fc domain. Tiragolumab is also described in WHO Drug Information (International Nonproprietary Names for Pharmaceutical Substances), Proposed INN: List 117, Vol. 31, No. 2, published Jul. 7, 2017 (see page 343).

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one aspect, the extent of binding of an anti-TIGIT antagonist antibody to an unrelated, non-TIGIT protein is less than about 10% of the binding of the antibody to TIGIT as measured, e.g., by a radioimmunoassay (RIA). In certain aspects, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-TIGIT antagonist antibody binds to an epitope of TIGIT that is conserved among TIGIT from different species or an epitope on TIGIT that allows for cross-species reactivity. In some aspects, the anti-TIGIT binding antibody has intact Fc-mediated effector function (e.g., tiragolumab, vibostolimab, etigilimab, EOS084448, or TJ-T6). In some aspects, the anti-TIGIT binding antibody has enhanced Fc-mediated effector function (e.g., SGN-TGT). In other aspects, the anti-TIGIT binding antibody lacks Fc-mediated effector function (e.g., domvanalimab, BMS-986207, ASP8374, or COM902). In some aspects, the anti-TIGIT binding antibody is an IgG1 class antibody (e.g., tiragolumab, vibostolimab, domvanalimab, BMS-986207, etigilimab, BGB-A1217, SGN-TGT, EOS084448 (EOS-448), TJ-T6, or AB308). In other aspects, the anti-TIGIT binding antibody is an IgG4 class antibody (e.g., ASP8374 or COM902). In one aspect, the anti-TIGIT antagonist antibody is tiragolumab.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1) (SEQ ID NO: 32). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is gastric cancer. In another instance, the cancer is rectal cancer. The cancer may be locally advanced or metastatic. In some instances, the cancer is locally advanced. In other instances, the cancer is metastatic. In some instances, the cancer may be unresectable (e.g., unresectable locally advanced or metastatic cancer). Examples of cancer include, but are not limited to, carcinoma, lymphoma, blastoma, sarcoma, and leukemia or lymphoid malignancies. More particular examples of cancers include, but are not limited to, gastric or stomach cancer, including gastrointestinal cancer (e.g., gastric carcinoma (GC) or a gastroesophageal junction carcinoma (GEJC) (e.g., inoperable, locally advanced, metastatic, or advanced GC or GEJC; gastric adenocarcinoma or gastroesophageal junction adenocarcinoma (e.g., adenocarcinoma of the esophagogastric junction)) or rectal cancer (e.g., locally advanced rectal cancer (LARC); adenocarcinoma of the rectum).

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis binding antagonist and one or more additional therapeutic agents, e.g., an anti-TIGIT antagonist antibody, e.g., tiragolumab and/or a chemotherapeutic agent (e.g., a platinum-based chemotherapeutic agent (e.g., oxaliplatin) and/or fluoropyrimidine-based chemotherapy agent (e.g., capecitabine or 5-fluorouracil (5-FU)))). Treating herein includes, inter alia, adjuvant therapy, neoadjuvant therapy, non-metastatic cancer therapy (e.g., locally advanced cancer therapy), and metastatic cancer therapy. In some aspects, treating comprises a neoadjuvant therapy (e.g., neoadjuvant chemoradiotherapy (nCRT)) followed by treatment with a PD-1 axis binding antagonist (e.g., atezolizumab) and an anti-TIGIT antagonist antibody, e.g., tiragolumab. In some embodiments, the treating further comprises surgery. The treatment may be first-line treatment (e.g., the subject may be previously untreated or not have received prior systemic therapy), or second line or later treatment.

Herein, an “effective amount” refers to the amount of a therapeutic agent (e.g., a PD-1 axis binding antagonist (e.g., atezolizumab) or a combination of therapeutic agents (e.g., a PD-1 axis binding antagonist and an anti-TIGIT antagonist antibody, e.g., atezolizumab and tiragolumab) and/or a chemotherapeutic agent (e.g., a platinum-based chemotherapeutic agent (e.g., oxaliplatin) and/or fluoropyrimidine-based chemotherapy agent (e.g., capecitabine or 5-fluorouracil (5-FU)))), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), and/or progression-free survival (PFS) and/or overall survival (OS)), and/or improved duration of response (DOR). Improvement (e.g., in terms of response rate (e.g., ORR, CR, and/or PR), survival (e.g., PFS and/or OS), or DOR) may be relative to a suitable reference treatment, for example, treatment that does not include the anti-TIGIT antagonist antibody (e.g., tiragolumab).

As used herein, “complete response” and “CR” refers to disappearance of all target lesions. For example, a reduction in the short axis of any pathological lymph nodes to <10 mm may be required for a CR.

As used herein, “pathological complete response” and “pCR” refer to the absence of residual invasive cancer on hematoxylin and eosin evaluation of tissue samples (e.g., surgery-resected samples) removed from primary tumor and lymph nodes after treatment with anti-cancer therapy (e.g., radiation, chemotherapy, immunotherapy, or neoadjuvant therapy). For example, pCR may be defined as no evidence of vital residual tumor cells by hematoxylin and eosin evaluation of the complete resected specimen and all sampled regional lymph nodes following completion of neoadjuvant therapy (ypT0N0 in the current AJCC staging system, 8th edition).

As used herein, “pathological complete response rate” and “pCR rate” refer to the proportion of subjects achieving pathological complete response. For example, the pCR rate may be defined as the proportion of subjects achieving pCR in surgery-resected samples evaluated by the local pathologist at each study site.

As used herein, the “sum of diameters” refers to the longest diameter for non-lymph node lesions (e.g., target lesions) and the short axis for lymph node lesions. For example, the sum of diameters may defined as the sum of all diameters for all target lesions, which may be calculated at baseline and at each tumor assessment.

As used herein, “partial response” and “PR” refers to at least a 30% decrease in the sum of diameters of all target lesions, taking as reference the baseline sum of diameters, in the absence of CR.

As used here, “progressive disease” and “PD” refers to at least a 20% increase in the sum of diameters of target lesions, taking as reference the smallest sum of diameters at prior timepoints, including baseline. The appearance of one or more new lesions may also be considered PD. For example, an absolute increase in the sum of diameters by ≥5 mm may also be required to demonstrate PD.

As used herein, “stable disease” and “SD” refer to neither sufficient shrinkage of target lesions to qualify for CR or PR nor sufficient increase to qualify for PD.

As used herein, “disease control rate” and “DCR” refer to the percentage of subjects who have achieved CR, PR, and stable disease (SD). For example, DCR refers to the proportion of pateints with stable disease for ≥12 weeks, a PR, or a CR, as determined by the investigator according to RECIST v1.1.

As used herein, “overall response rate,” “objective response rate,” and “ORR” refer interchangeably to the sum of CR rate and PR rate. For example, ORR may be defined as the proportion of subjects with a CR or a PR, as determined by the investigator according to RECIST v1.1. In another example, ORR may be defined as the proportion of subjects with a CR or a PR on two consecutive occasions ≥4 weeks apart, as determined by the investigator according to RECIST v1.1.

As used herein, “complete clinical response rate” or “cCR rate” refers to the proportion of patients with no local evidence of residual tumor (yield clinical T0N0, ycT0N0) after neoadjuvant chemoradiotherapy assessed by endoscopy MRI and physical examination.

As used herein, “progression-free survival” and “PFS” refer to the length of time during and after treatment during which the cancer does not get worse. PFS may include the amount of time subjects have experienced a CR or a PR, as well as the amount of time subjects have experienced stable disease. For example, PFS may be defined as the time from randomization to the first occurrence of disease progression or death from any cause, whichever occurs first, as determined by the investigator according to RECIST v1.1.

As used herein, “overall survival” and “OS” refer to the length of time from either the date of diagnosis or the start of treatment for a disease (e.g., cancer) that the subject is still alive. For example, OS may be defined as the defined as the time from randomization to death due to any cause. OS may be defined at specific timepoints (e.g., at 6 or 12 months).

As used herein, the term “duration of response” and “DOR” refer to a length of time from documentation of a tumor response until disease progression or death from any cause, whichever occurs first. For example, DOR may be defined as the time from the first occurrence of a documented objective response to disease progression or death from any cause, whichever occurs first, per RECIST v1.1 as determined by the investigator. DOR may be derived for subjects with a CR or a PR.

As used herein, “R0 resection rate” refers to the proportion of subjects with a microscopically margin-negative resection, in which no gross or microscopic tumor remains in the primary tumor bed and/or sampled regional lymph nodes based on evaluation by a pathologist.

As used herein, “relapse-free survival rate” and “RFS rate” refer to the proportion of subjects who have not experienced disease relapse or death from any cause at a certain timepoint. For example, RFS rate may be defined as the proportion of subjects who have not experienced disease relapse or death from any cause at one, two or three year(s), as determined by the investigator.

As used herein, “event-free survival rate” and “EFS rate” refer to the proportion of subjects who have not experienced certain events after randomization at a certain timepoint. For example, EFS rate may be defined as the proportion of subjects who have not experienced certain events (e.g., progression of disease that precludes surgery, local or distant recurrence, or death due to any cause) at one, two or three year(s) after randomization.

As used herein, the term “chemotherapeutic agent” refers to a compound useful in the treatment of cancer, such as gastric carcinoma (GC), gastroesophageal junction carcinoma (GEJC), or rectal cancer. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chlorambucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretinoic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “patient” refers to a human patient or subject. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an immunohistochemical (IHC) assay, e.g., an IHC assay staining for PD-L1 using the antibody SP142, SP263, 22C3, or 28-8. Accordingly, a PD-L1-positive tumor cell fraction may be calculated using the PD-L1 IHC SP142 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively.

As used herein, the “Ventana SP142 IHC assay” is conducted according to the Ventana PD-L1 (SP142) Assay package insert (Tucson, AZ: Ventana Medical Systems, Inc.), which is incorporated herein by reference in its entirety.

As used herein, the “Ventana SP263 IHC assay” is conducted according to the Ventana PD-L1 (SP263) Assay package insert (Tucson, AZ: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, CA: Dako, Agilent Pathology Solutions), which is incorporated herein by reference in its entirety.

As used herein, the “pharmDx 28-8 IHC assay” is conducted according to the PD-L1 IHC 28-8 pharmDx package insert (Carpinteria, CA: Dako, Agilent Pathology Solutions), which is incorporated herein by reference in its entirety.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and an anti-TIGIT antagonist antibody (e.g., tiragolumab) or a treatment regimen that includes administration of a PD-1 axis binding antagonist (e.g., atezolizumab), an anti-TIGIT antagonist antibody (e.g., tiragolumab), and one or more chemotherapeutic agents (e.g., a platinum-based chemotherapeutic agent (e.g., oxaliplatin) and/or fluoropyrimidine-based chemotherapy agent (e.g., capecitabine or 5-fluorouracil (5-FU))). In some examples, the treatment regimen also includes neoadjuvant chemoradiotherapy. As such, “in combination with” refers to administration of one treatment modality before, during, or after administration of the other treatment modality to the subject.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v4.0 or v5.0 (NIH CTCAE). In some aspects, the AE is a low-grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high-grade AE, e.g., a Grade 3, Grade 4, or Grade 5 AE. In some instances, the AE is a Grade 3 or a Grade 4 AE. Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “treatment-related AE” refers to an AE that is judged by an investigator to have occurred as a result of a treatment, e.g., a PD-1 axis binding antagonist therapy (e.g., atezolizumab therapy) and/or an anti-TIGIT antagonist antibody therapy (e.g., tiragolumab therapy).

### II. Therapeutic Methods and Compositions for Cancer

Provided herein are methods and uses for treating cancer (e.g., gastric carcinoma (GC) or a gastroesophageal junction carcinoma (GEJC) (e.g., inoperable, locally advanced, metastatic, or advanced GC or GEJC), or rectal cancer (e.g., locally advanced rectal cancer (LARC))) in a subject or population of subjects comprising administering to the subject or population of subjects one or more dosing cycles of an effective amount of a combination of both an anti-TIGIT antagonist antibody (e.g., tiragolumab) and an anti-PD-L1 antagonist antibody (e.g., atezolizumab).

### A. Therapeutic Methods and Uses Relating to Gastric Carcinoma and Gastroesophageal Carcinoma

i. Methods for Treating Gastric Carcinoma and Gastroesophageal Carcinoma

In one aspect, provided herein is a method for treating a subject or population of subjects having a gastric carcinoma (GC) or a gastroesophageal junction carcinoma (GEJC) (e.g., an inoperable, locally advanced, metastatic, or advanced GC or GEJC), comprising administering to the subject or population of subjects a dosing regimen comprising one or more dosing cycles of an anti-TIGIT antagonist antibody (e.g., tiragolumab), a PD-1 axis binding antagonist (e.g., atezolizumab), capecitabine, and oxaliplatin. Exemplary anti-TIGIT antagonist antibodies are provided in Section V. Exemplary PD-1 axis binding antagonists are provided in Section VI.

In some instances, a subject or a population of subjects receiving an anti-TIGIT antagonist antibody (e.g., an anti-TIGIT antagonist antibody as disclosed herein, e.g., tiragolumab), a PD-1 axis binding antagonist (e.g., anti-PD-L1 antagonist antibody, such as atezolizumab), and a chemotherapy (e.g., a platinum agent (e.g., oxaliplatin) and/or one or more fluoropyrimidine-based chemotherapy agents (e.g., capecitabine or 5-fluorouracil (5-FU))) is being treated for a GC or a GEJC (e.g., inoperable, locally advanced, metastatic, or advanced GC or GEJC). In some instances, the anti-TIGIT antagonist antibody is tiragolumab. In some instances, the PD-1 axis binding antagonist is an anti-PD-L1 antagonist antibody. In some instances, the anti-PD-L1 antagonist antibody is atezolizumab. In some instances, the platinum agent is oxaliplatin. In some instances, the fluoropyrimidine-based chemotherapy agent is capecitabine. In some instances, the fluoropyrimidine-based chemotherapy agent is 5-FU. In some instances, the chemotherapy is capecitabine and oxaliplatin (CAPOX). In some instances, the GC is gastric adenocarcinoma. In some instances, the GEJC is gastroesophageal junction adenocarcinoma (e.g., adenocarcinoma of the esophagogastric junction).

In some aspects, the GC or GEJC is human epidermal growth factor receptor 2 (HER2)-negative.

In some aspects, the GC or GEJC is an adenocarcinoma.

In some aspects, the subject or population of subjects has not received prior systemic therapy for GC or GEJC. In some embodiments, the subject or population of subjects has not received a prior treatment for the non-advanced GC or GEJC comprising a chemoradiotherapy or a chemotherapy (e.g., chemoradiotherapy or chemotherapy administered with curative intent or in an adjuvant or neoadjuvant setting).

In some aspects, the method comprises administering to the subject or population of subjects: (a) the anti-TIGIT antagonist antibody at a fixed dose of about 600 mg every three weeks; (b) the PD-1 axis binding antagonist at a fixed dose of about 1200 mg every three weeks; (c) capecitabine at a dose of 1000 mg/m² twice daily for two weeks; and (d) oxaliplatin at a dose of 130 mg/m² every three weeks.

In some aspects, the length of each of the one or more dosing cycles is 21 days.

In some aspects, the method comprises administering to the subject or population of subjects the anti-TIGIT antagonist antibody (e.g., tiragolumab), the PD-1 axis binding antagonist (e.g., atezolizumab), and oxaliplatin on about Day 1 of each of the one or more dosing cycles.

Other exemplary dosing regimens for anti-TIGIT antagonist antibodies and PD-1 axis binding antagonists are provided in Section IIC. In some aspects, the anti-TIGIT antagonist antibody and/or the PD-1 axis binding antagonist are administered in a dosing regimen provided in Section IIC.

In some embodiments, oxaliplatin is administered once per week, once every two weeks, once every three weeks, twice every three weeks, once every four weeks, twice every four weeks, or three times every four weeks. In some embodiments, capecitabine and oxaliplatin are administered in a 21-day cycle with capecitabine administered twice daily on Days 1-14 and oxaliplatin administered on Day 1 of each cycle.

In some aspects, the method comprises administering to the subject or population of subjects capecitabine on Days 1-14 of each of the one or more dosing cycles. In some aspects, the method comprises administering to the subject or population of subjects the capecitabine orally.

In some embodiments, capecitabine is administered once daily for one week, twice daily for one week, or three times daily for one week. In some embodiments, capecitabine is administered once daily for two weeks, twice daily for two weeks, or three times daily for two weeks. In some embodiments, capecitabine is administered in a 21-day cycle. For example, in a 21-day cycle, capecitabine may be administered twice daily for two weeks, followed by 1 week of rest.

In some aspects, the method comprises administering to the subject or population of subjects the PD-1 axis binding antagonist (e.g., atezolizumab) before the anti-TIGIT antagonist antibody (e.g., tiragolumab). In some embodiments, the method comprises administering to the subject or population of subjects the anti-TIGIT antagonist antibody before the PD-1 axis binding antagonist. In some embodiments, capecitabine is administered after the PD-1 axis binding antagonist and the anti-TIGIT antagonist antibody. In some embodiments, oxaliplatin is administered after the PD-1 axis binding antagonist and the anti-TIGIT antagonist antibody. In some embodiments, capecitabine and oxaliplatin are administered after the PD-1 axis binding antagonist and the anti-TIGIT antagonist antibody.

In some aspects, the method comprises administering to the subject or population of subjects the anti-TIGIT antagonist antibody (e.g., tiragolumab), the PD-1 axis binding antagonist (e.g., atezolizumab), and the oxaliplatin intravenously.

In some aspects, the treating results in an increase in objective response rate (ORR) as compared to a reference ORR. In some aspects, the reference ORR is an ORR of a population of subjects who have received: (a) a treatment comprising capecitabine and oxaliplatin and not comprising a PD-1 axis binding antagonist (e.g., atezolizumab) and an anti-TIGIT antagonist antibody (e.g., tiragolumab); and/or (b) a treatment comprising capecitabine, oxaliplatin, and a PD-1 axis binding antagonist (e.g., atezolizumab) and not comprising an anti-TIGIT antagonist antibody (e.g., tiragolumab).

In some aspects, the treating results in an increase in progression-free survival (PFS) as compared to a reference PFS. In some aspects, the reference PFS is a median PFS of a population of subjects who have received: (a) a treatment comprising capecitabine and oxaliplatin and not comprising a PD-1 axis binding antagonist (e.g., atezolizumab) and an anti-TIGIT antagonist antibody (e.g., tiragolumab); and/or (b) a treatment comprising capecitabine, oxaliplatin, and a PD-1 axis binding antagonist (e.g., atezolizumab) and not comprising an anti-TIGIT antagonist antibody (e.g., tiragolumab).

In some aspects, the treating results in an increase in overall survival (OS) as compared to a reference OS. In some aspects, the reference OS is a median OS of a population of subjects who have received: (a) a treatment comprising capecitabine and oxaliplatin and not comprising a PD-1 axis binding antagonist (e.g., atezolizumab) and an anti-TIGIT antagonist antibody (e.g., tiragolumab); and/or (b) a treatment comprising capecitabine, oxaliplatin, and a PD-1 axis binding antagonist (e.g., atezolizumab) and not comprising an anti-TIGIT antagonist antibody (e.g., tiragolumab).

In some aspects, the treating results in an increase in duration of response (DOR) as compared to a reference DOR. In some aspects, the reference DOR is a median DOR of a population of subjects who have received: (a) a treatment comprising capecitabine and oxaliplatin and not comprising a PD-1 axis binding antagonist (e.g., atezolizumab) and an anti-TIGIT antagonist antibody (e.g., tiragolumab); and/or (b) a treatment comprising capecitabine, oxaliplatin, and a PD-1 axis binding antagonist (e.g., atezolizumab) and not comprising an anti-TIGIT antagonist antibody (e.g., tiragolumab).

In some embodiments, surgery is unsuitable for the subject or population of subjects.

ii. Prior Therapy

In some embodiments, the subject has not been previously treated with an anti-cancer therapy (e.g., a cancer immunotherapy and/or a chemotherapeutic agent) for the cancer (e.g., GC or GEJC, e.g., inoperable, locally advanced, metastatic, or advanced GC or GEJC). In some embodiments, the subject has received prior treatment with an anti-cancer therapy (e.g., a cancer immunotherapy and/or a chemotherapeutic agent) for the cancer (e.g., GC or GEJC, e.g., inoperable, locally advanced, metastatic, or advanced GC or GEJC). In some instances, the subject has received at least one line of prior therapy. In some instances, the subject has received two or more prior anti-cancer therapies for the cancer (e.g., GC or GEJC). In some instances, the subject has received three or more prior anti-cancer therapies for the cancer (e.g., GC or GEJC). In some instances, the subject has received two lines of prior therapy. In some instances, the subject has received three lines of prior therapy. In some instances, the subject has received four lines of prior therapy. In some instances, the subject has received more than four lines of prior therapy. In some instances, the subject experienced disease progression during or following treatment with the prior anti-cancer therapy. In some instances, the prior therapy is chemotherapy, surgery, and/or radiotherapy.

In some instances, the subject has not received prior systemic therapy (e.g., prior systemic therapy with curative intent, e.g., chemotherapy) within at least the month prior to the administration with the PD-1 axis binding antagonist and the anti-TIGIT antagonist antibody (e.g., within the two months prior, three months prior, four months prior, six months prior, one year prior, two years prior, three years prior, four years prior, five years prior, or ten years prior to the administration with the PD-1 axis binding antagonist and the anti-TIGIT antagonist antibody). In some instances, the subject is chemotherapy naïve. In some instances, the subject has not received prior immunotherapy.

iii. Lack of Treatment-Related Adverse Events

In some embodiments, the subject does not experience a treatment-related adverse event (AE) (e.g., a Grade 1, Grade 2, Grade 3, or Grade 4 treatment-related adverse event) during or following the one or more dosing cycles of tiragolumab and atezolizumab. In some embodiments, the subject experiences a treatment-related Grade 1 or Grade 2 adverse event during or following the one or more dosing cycles of tiragolumab and atezolizumab. In some embodiments, the subject does not experience a treatment-related Grade 3 or Grade 4 adverse event during or following the one or more dosing cycles of tiragolumab and atezolizumab. Treatment-related adverse events include, e.g., tiragolumab-related adverse events and/or atezolizumab-related adverse events. Adverse events are graded according to the National Cancer Institute Common Terminology Criteria for Adverse Events (NCI CTCAE), Version 4.0.

Causality of adverse events (e.g., determination of whether an adverse event is treatment-related) may be based on the following guidance:
Temporal relationship of event onset to the initiation of study drug.
Course of the event, considering especially the effects of dose reduction, discontinuation of study drug, or reintroduction of study drug (as applicable).
Known association of the event with the study drug or with similar treatments.
Known association of the event with the disease under study.
Presence of risk factors in the subject or use of concomitant medications known to increase the occurrence of the event.
Presence of non-treatment-related factors that are known to be associated with the occurrence of the event.

In general, an adverse event may be attributed to the study drug (e.g., tiragolumab and/or atezolizumab) if there is a plausible temporal relationship between the onset of the adverse event and administration of the study drug, and the adverse event cannot be readily explained by the subject's clinical state, intercurrent illness, or concomitant therapies; and/or the adverse event follows a known pattern of response to the study drug; and/or the adverse event abates or resolves upon discontinuation of the study drug or dose reduction and, if applicable, reappears upon re-challenge.

An adverse event may be identified as non-treatment-related if evidence exists that the adverse event has an etiology other than the study drug (e.g., preexisting medical condition, underlying disease, intercurrent illness, or concomitant medication); and/or the adverse event has no plausible temporal relationship to administration of the study drug (e.g., cancer diagnosed 2 days after first dose of study drug).

Several potential risks exist for tiragolumab based on the mechanism of action, known effect of similar checkpoint inhibitors, and nonclinical data. As an antagonist of TIGIT, tiragolumab is anticipated to enhance T-cell and NK cell proliferation, survival, and function. Therefore, tiragolumab may increase the risk of autoimmune inflammation (also described as immune-mediated adverse events). In addition, due to the intact Fc-effector function of tiragolumab, lymphopenia via antibody-dependent cellular cytotoxicity (ADCC) is a theoretical risk. Particular adverse events associated with itagolumab include infusion-related reactions (IRRs), immune-mediated adverse events, and lymphopenia.

Atezolizumab has been associated with risks such as the following: IRRs and immune-mediated hepatitis, pneumonitis, colitis, pancreatitis, diabetes mellitus, hypothyroidism, hyperthyroidism, adrenal insufficiency, hypophysitis, Guillain-Barre syndrome, myasthenic syndrome or myasthenia gravis, meningoencephalitis, myocarditis, myositis and nephritis. Immune-mediated adverse reactions may involve any organ system and may lead to hemophagocytic lymphohistiocytosis (HLH) and macrophage activation syndrome (MAS).

In any of the preceding examples, each dosing cycle may have any suitable length, e.g., about 7 days, about 14 days, about 21 days, about 28 days, or longer. In some instances, each dosing cycle is about 21 days. In some instances, tiragolumab is administered every three weeks (e.g., on Day 1 of each 21-day dosing cycle) and atezolizumab is administered every three weeks (e.g., on Day 1 of each 21-day dosing cycle).

The subject is preferably a human.

iv. Responses to Treatment

In some embodiments of any of the methods described herein, the response to the treatment (e.g., atezolizumab and tiragolumab) of a subject or population of subjects having a GC or GEJC (e.g., an inoperable, locally advanced, metastatic, or advanced GC or GEJC) can be characterized by one or more measures. In some embodiments, the treatment results in an increase in PFS, OS, or DOR in the subject. In some embodiments, the treatment results in an increase in the ORR in the population of subjects. In some embodiments, the treatment results in disease control, SD, a CR, or a PR in the subject.

For example, in embodiments in which an anti-TIGIT antagonist antibody (e.g., tiragolumab) in combination with a PD-1 axis binding antagonist (e.g., atezolizumab), capecitabine, and oxaliplatin is administered, the treatment may result in an increase in PFS of the subject, e.g., as compared to the median PFS from a population of subjects treated with the PD-1 axis binding antagonist, capecitabine, and oxaliplatin without the anti-TIGIT antagonist antibody.

For example, in embodiments in which an anti-TIGIT antagonist antibody (e.g., tiragolumab) in combination with a PD-1 axis binding antagonist (e.g., atezolizumab), capecitabine, and oxaliplatin is administered, the treatment may result in an increase in OS of the subject, e.g., as compared to the median OS from a population of subjects treated with the PD-1 axis binding antagonist, capecitabine, and oxaliplatin without the anti-TIGIT antagonist antibody.

Progression-free survival of the subject can be measured according to RECIST v1.1 criteria, as described in Eisenhauer et al., Eur. J. Cancer. 2009, 45:228-47. PFS refers to the length of time during and after treatment during which a subject's cancer (e.g., a GC or GEJC cancer) does not get worse. PFS may include the amount of time subjects have experienced a CR, a PR, or SD.

In some embodiments, PFS is measured as the period of time from randomization to the first occurrence of disease progression or death from any cause as determined by RECIST v1.1 criteria. In some embodiments, PFS is measured as the time from randomization to the time of death. In some embodiments, PFS is measured as the time from the initiation of the stage of the study to the first occurrence of disease progression or death from any cause, whichever occurs first, as determined by RECIST v1.1 criteria. In some embodiments, a treatment described herein results in an increase in PFS as compared to a reference PFS by at least about 1 month (e.g., 1 month, 2 months, 3.0 months, 4.0 months, 5.0 months, 6.0 months, 7.0 months, 8.0 months, 9.0 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the reference PFS is a PFS of a population of subjects who have received a treatment comprising capecitabine and oxaliplatin and not comprising a PD-1 axis binding antagonist (e.g., atezolizumab) and an anti-TIGIT antagonist antibody (e.g., tiragolumab); and/or a treatment comprising capecitabine, oxaliplatin, and a PD-1 axis binding antagonist (e.g., atezolizumab) and not comprising an anti-TIGIT antagonist antibody (e.g., tiragolumab).

In some embodiments, a treatment described herein results in an increase in OS as compared to a reference OS by at least about 1 month (e.g., 1 month, 2 months, 3.0 months, 4.0 months, 5.0 months, 6.0 months, 7.0 months, 8.0 months, 9.0 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the reference OS is an OS of a population of subjects who have received a treatment comprising capecitabine and oxaliplatin and not comprising a PD-1 axis binding antagonist and an anti-TIGIT antagonist antibody; and/or a treatment comprising capecitabine, oxaliplatin, and a PD-1 axis binding antagonist and not comprising an anti-TIGIT antagonist antibody.

In some embodiments of any of the methods described herein, a population of subjects' response to the treatment (e.g., atezolizumab, tiragolumab, capecitabine, and oxaliplatin) can be characterized by one or more measures.

In some instances, the treatment results in an increase in ORR as compared to a reference ORR in a population of subjects, e.g., as compared to a treatment comprising capecitabine and oxaliplatin and not comprising a PD-1 axis binding antagonist and an anti-TIGIT antagonist antibody, and/or as compared to a treatment comprising capecitabine, oxaliplatin, and a PD-1 axis binding antagonist and not comprising an anti-TIGIT antagonist antibody. For example, in embodiments in which an anti-TIGIT antagonist antibody (e.g., tiragolumab) in combination with a PD-1 axis binding antagonist (e.g., atezolizumab), capecitabine, and oxaliplatin is administered, the treatment may result in an increase in ORR of the population of subjects, e.g., as compared to a reference ORR from a population of subjects treated with the PD-1 axis binding antagonist, capecitabine, and oxaliplatin without the anti-TIGIT antagonist antibody. In some embodiments, a treatment described herein results in an increase in ORR as compared to a reference ORR by at least about 1% (e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%). In some embodiments, the reference ORR is an ORR of a population of subjects who have received a treatment comprising capecitabine and oxaliplatin and not comprising a PD-1 axis binding antagonist and an anti-TIGIT antagonist antibody; and/or a treatment comprising capecitabine, oxaliplatin, and a PD-1 axis binding antagonist and not comprising an anti-TIGIT antagonist antibody.

In some aspects, the clinical response to the treatment is a reduction in the sum of diameters of one or more target lesions (e.g., GC or GEJC tumors). In some aspects, the sum of diameters is decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or the sum of diameters is decreased by 100% (e.g., target lesions disappear) during or following administration of the one or more dosing cycles of an anti-TIGIT antagonist antibody (e.g., tiragolumab) and a PD-1 axis binding antagonist (e.g., atezolizumab), e.g., is decreased relative to a measurement taken before administration of the one or more dosing cycles of tiragolumab and atezolizumab.

In some aspects, the treating results in a clinical response that is maintained for at least 1 month, at least 2 months, at least 3 months, at least 4 months, at least 5 months, at least 6 months, at least 7 months, at least 8 months, at least 9 months, at least 10 months, at least 11 months, at least 1 year, at least 1 year and 1 month, at least 1 year and 2 months, at least 1 year and 3 months, at least 1 year and 4 months, at least 1 year and 5 months, at least 1 year and 6 months, at least 1 year and 7 months, at least 1 year and 8 months, at least 1 year and 9 months, at least 1 year and 10 months, at least 1 year and 11 months, at least 2 years, at least 2 years and 1 month, at least 2 years and 2 months, at least 2 years and 3 months, at least 2 years and 4 months, at least 2 years and 5 months, at least 2 years and 6 months, at least 2 years and 7 months, at least 2 years and 8 months, at least 2 years and 9 months, at least 2 years and 10 months, at least two years and 11 months, at least 3 years, at least 3.5 years, at least 4 years, at least 4.5 years, at least 5 years, at least 5.5 years, at least 6 years, at least 6.5 years, at least 7 years, at least 7.5 years, at least 8 years, at least 8.5 years, at least 9 years, at least 9.5 years, at least 10 years, or more than 10 years (e.g., the clinical response is maintained for 1-2 months, 2-4 months, 4-6 months, 6-8 months, 8-10 months, 10-12 months, 1 year to 1.5 years, 1.5 years to 2 years, 2 years to 2.5 years, 2.5 years to 3 years, 3 years to 3.5 years, 3.5 years to 4 years, 4 years to 4.5 years, 4.5 years-5 years, 5 years to 6 years, 6 years to 7 years, 7 years to 8 years, 8 years to 9 years, or 9 years to 10 years). For example, in some aspects, the clinical response is maintained for 1 month to 10 years, 6 months to 5 years, 1 year to 4 years, 1 year to 3 years, or 1 year to 2 years.

In some aspects, the treating results in a clinical response that is maintained for at least 1 year. In some aspects, the clinical response is maintained for at least 2 years.

### B. Therapeutic Methods and Uses Relating to Rectal Cancer

i. Methods for Treating Rectal Cancer

In one aspect, provided herein is a method for treating a subject or population of subjects having a rectal cancer (e.g., locally advanced rectal cancer (LARC)), comprising administering to the subject or population of subjects a dosing regimen comprising one or more dosing cycles of an anti-TIGIT antagonist antibody (e.g., tiragolumab) and a PD-1 axis binding antagonist (e.g., an anti-PD-L1 antagonist antibody, e.g., atezolizumab), wherein the one or more dosing cycles are performed following a neoadjuvant chemotherapy (nCRT) regimen. In some embodiments, the rectal cancer is a resectable

LARC. Exemplary anti-TIGIT antagonist antibodies are provided in Section V. Exemplary PD-1 axis binding antagonists are provided in Section VI.

In some aspects, the rectal cancer is a stage cT₃N+M₀ or stage cT₄N_{any}M₀ rectal cancer. In some embodiments, the rectal cancer is a resectable LARC with a clinical stage of cT₃N+M₀ or cT₄N_{any}M₀.

In some aspects, the rectal cancer is an adenocarcinoma.

In some aspects, the subject or population of subjects does not have synchronous colon cancer.

In some aspects, the subject or population of subjects has not received a prior therapy for rectal cancer.

In some aspects, the method comprises administering to the subject or population of subjects the anti-TIGIT antagonist antibody (e.g., tiragolumab) at a fixed dose of about 600 mg every three weeks and the PD-1 axis binding antagonist (e.g., atezolizumab) at a fixed dose of about 1200 mg every three weeks.

In some aspects, the length of each of the one or more dosing cycles is 21 days.

In some aspects, the method comprises administering to the subject or population of subjects the anti-TIGIT antagonist antibody (e.g., tiragolumab) and the PD-1 axis binding antagonist (e.g., atezolizumab) on about Day 1 of each of the one or more dosing cycles.

Other exemplary dosing regimens for anti-TIGIT antagonist antibodies and PD-1 axis binding antagonists are provided in Section IIC. In some aspects, the anti-TIGIT antagonist antibody and/or the PD-1 axis binding antagonist are administered in a dosing regimen provided in Section IIC.

In some aspects, the method comprises administering to the subject or population of subjects the PD-1 axis binding antagonist (e.g., atezolizumab) before the anti-TIGIT antagonist antibody (e.g., tiragolumab).

In some aspects, the method comprises administering to the subject or population of subjects the anti-TIGIT antagonist antibody (e.g., tiragolumab) and PD-1 axis binding antagonist (e.g., atezolizumab) intravenously.

In some aspects, the one or more dosing cycles are initiated about two weeks after the last cycle of nCRT.

In some aspects, the one or more dosing cycles are initiated within four weeks after the last cycle of nCRT.

In some aspects, the nCRT regimen comprises radiotherapy delivered to the pelvis at a fraction of about 1.8 Gy per treatment. In some aspects, the radiotherapy is administered on Days 1-5 every week.

In some aspects, the nCRT regimen comprises administering a total of between about 45 and about 50.4 Gy of the radiotherapy to the subject or population of subjects.

In some aspects, the radiotherapy is administered in 25 to 28 fractions.

In some aspects, the nCRT regimen comprises a fluoropyrimidine-based chemotherapy.

In some aspects, the fluoropyrimidine-based chemotherapy is capecitabine or 5-fluorouracil (5-FU).

In some aspects, the capecitabine is administered orally at a dose of about 825 mg/m².

In some aspects, the capecitabine is administered orally twice daily on five consecutive days every week.

In some aspects, the capecitabine is administered orally twice daily on seven consecutive days every week.

In some aspects, the 5-FU is administered intravenously at a dose of about 225 mg/m².

In some aspects, the 5-FU is administered on five consecutive days every week.

In some aspects, the 5-FU is administered on seven consecutive days every week.

In some aspects, the nCRT is performed for 5 cycles.

In some aspects, the first dosing cycle of the anti-TIGIT antagonist antibody (e.g., tiragolumab) and PD-1 axis binding antagonist (e.g., atezolizumab) is initiated prior to a surgery.

In some aspects, three dosing cycles are completed prior to the surgery.

In some aspects, the surgery is performed within about four weeks after the last dosing cycle.

In some aspects, the surgery is radical surgical resection using total mesorectal excision (TME) and lymph node dissection.

In some aspects, the treating results in a pathological complete response (pCR) and/or an increase in pCR rate as compared to a reference pCR rate. In some aspects, the reference pCR rate is a pCR rate of population of subjects who have received a treatment comprising: (a) nCRT not followed by treatment with a PD-1 axis binding antagonist (e.g., atezolizumab) and an anti-TIGIT antagonist antibody (e.g., tiragolumab); and/or (b) nCRT followed by treatment with a PD-1 axis binding antagonist (e.g., atezolizumab).

In some aspects, the treating results in an increase in R0 resection rate as compared to a reference R0 resection rate. In some aspects, the reference R0 resection rate is an R0 resection rate of a population of subjects who have received a treatment comprising: (a) nCRT not followed by treatment with a PD-1 axis binding antagonist (e.g., atezolizumab) and an anti-TIGIT antagonist antibody (e.g., tiragolumab); and/or (b) nCRT followed by treatment with a PD-1 axis binding antagonist (e.g., atezolizumab).

In some aspects, the treating results in an increase in objective response rate (ORR) as compared to a reference ORR. In some aspects, the reference ORR is an ORR of a population of subjects who have received a treatment comprising: (a) nCRT not followed by treatment with a PD-1 axis binding antagonist (e.g., atezolizumab) and an anti-TIGIT antagonist antibody (e.g., tiragolumab); and/or (b) nCRT followed by treatment with a PD-1 axis binding antagonist (e.g., atezolizumab).

In some aspects, the treating results in an increase in relapse-free survival (RFS) rate as compared to a reference RFS rate. In some aspects, the reference RFS rate is an RFS rate of a population of subjects who have received a treatment comprising: (a) nCRT not followed by treatment with a PD-1 axis binding antagonist (e.g., atezolizumab) and an anti-TIGIT antagonist antibody (e.g., tiragolumab); and/or (b) nCRT followed by treatment with a PD-1 axis binding antagonist (e.g., atezolizumab). In some aspects, the RFS rate is a one-year RFS rate, a two-year RFS rate, or a three-year RFS rate.

In some aspects, the treating results in an increase in event-free survival (EFS) rate as compared to a reference EFS rate. In some aspects, the reference EFS rate is an EFS rate of a population of subjects who have received a treatment comprising: (a) nCRT not followed by treatment with a PD-1 axis binding antagonist (e.g., atezolizumab) and an anti-TIGIT antagonist antibody (e.g., tiragolumab); and/or (b) nCRT followed by treatment with a PD-1 axis binding antagonist (e.g., atezolizumab). In some aspects, the EFS rate is a one-year RFS rate, a two-year EFS rate, or a three-year EFS rate.

In one aspect, the invention provides a method for treating a subject or population of subjects having a rectal cancer (e.g., LARC), wherein the method comprises administering to the subject or population of subjects one or more dosing cycles of anti-TIGIT antagonist antibody (e.g., at a fixed dose of about 30 mg to about 1200 mg every three weeks (e.g., at a fixed dose of about 30 mg to about 800 mg every three weeks, e.g., at a fixed dose of about 600 mg every three weeks)) and a PD-1 axis binding antagonist (e.g., at a fixed dose of about 80 mg to about 1600 mg every three weeks (e.g., at a fixed dose of about 800 mg to about 1400 mg, e.g., at a fixed dose of about 1200 mg)).

In some embodiments, the subject or population of subjects has received no prior therapy for rectal cancer (e.g., LARC). In some embodiments, the subject or population of subjects has received no prior systemic treatment for rectal cancer (e.g., LARC). In some embodiments, the subject or population of subjects has received no prior local-regional treatment for rectal cancer (e.g., LARC). In some embodiments, the subject or population of subjects has received no prior local-regional and systemic treatment for rectal cancer (e.g., LARC).

In some embodiments, the anti-TIGIT antagonist antibody is administered at a fixed dose of about 600 mg every three weeks and the PD-1 axis binding antagonist is administered at a fixed dose of about 1200 mg every three weeks. In some embodiments, the dosing cycle comprises 21 days. In some embodiments, the one or more dosing cycles are initiated about two weeks after the last cycle of nCRT. In some embodiments, the one or more dosing cycles are initiated within four weeks after the last cycle of nCRT.

In some embodiments, the nCRT regimen comprises radiotherapy delivered to the pelvis at a fraction of about 1.8 Gy per treatment. In some embodiments, the radiotherapy is administered on Days 1-5 every week. In some embodiments, the nCRT regimen comprises administering a total of between about 45 and about 50.4 Gy of the radiotherapy to the subject or population of subjects.

In some embodiments, the radiotherapy is administered in about 25 to 28 fractions (e.g., 15 to 35 fractions, 15 to 33 fractions, 15 to 31 fractions, 15 to 29 fractions, 15 to 27 fractions, 15 to 25 fractions, 15 to 23 fractions, 15 to 21 fractions, 15 to 19 fractions, 15 to 17 fractions, 17 to 35 fractions, 19 to 35 fractions, 21 to 35 fractions, 23 to 35 fractions, 25 to 35 fractions, 27 to 35 fractions, 29 to 35 fractions, 31 to 35 fractions, 33 to 35 fractions, 17 to 33 fractions, 19 to 31 fractions, 21 to 29 fractions, 23 to 27 fractions, or 25 to 27 fractions). In some embodiments, the radiotherapy is administered in 25 to 28 fractions.

In some embodiments, the nCRT regimen comprises a fluoropyrimidine-based chemotherapy. In some embodiments, the fluoropyrimidine-based chemotherapy is capecitabine or 5-fluorouracil (5-FU). In some embodiments, the capecitabine is administered orally at a dose of about 825 mg/m². In some embodiments, the capecitabine is administered orally twice daily on five consecutive days every week. In some embodiments, the capecitabine is administered orally twice daily on seven consecutive days every week. In some embodiments, the 5-FU is administered intravenously at a dose of about 225 mg/m². In some embodiments, the 5-FU is administered on five consecutive days every week. In some embodiments, the 5-FU is administered on seven consecutive days every week. In some embodiments, the nCRT is performed for 5 cycles.

In some embodiments, the first dosing cycle of the anti-TIGIT antagonist antibody and PD-1 axis binding antagonist is initiated prior to a surgery. In some embodiments, three dosing cycles are completed prior to the surgery. In some embodiments, the surgery is performed within about four weeks after the last dosing cycle. In some embodiments, the surgery is radical surgical resection using total mesorectal excision (TME) and lymph node dissection.

In some embodiments, the method comprises administering to the subject or population of subjects the anti-TIGIT antagonist antibody and the PD-1 axis binding antagonist intravenously. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks) and the PD-1 axis binding antagonist (e.g., an anti-PD-L1 antagonist antibody (e.g., atezolizumab)) is administered intravenously at a fixed dose of about 1200 mg on Day 1 of each 21-day cycle (i.e., at a fixed dose of about 1200 mg every three weeks).

In some embodiments, the method comprises administering to the subject or population of subjects the PD-1 axis binding antagonist before the anti-TIGIT antagonist antibody. In some embodiments, the method comprises administering to the subject or population of subjects the anti-TIGIT antagonist antibody before the PD-1 axis binding antagonist.

In some aspects, the subject is a human.

ii. Prior Therapy

In some embodiments, the subject has not been previously treated with an anti-cancer therapy (e.g., a cancer immunotherapy and/or a chemotherapeutic agent) for the cancer (e.g., rectal cancer, e.g., locally advanced rectal cancer (LARC)). In some embodiments, the subject has received prior treatment with an anti-cancer therapy (e.g., a cancer immunotherapy and/or a chemotherapeutic agent) for the cancer (e.g., rectal cancer, e.g., LARC). In some instances, the subject has received at least one line of prior therapy. In some instances, the subject has received two or more prior anti-cancer therapies for the cancer (e.g., rectal cancer, e.g., LARC). In some instances, the subject has received three or more prior anti-cancer therapies for the cancer (e.g., rectal cancer, e.g., LARC). In some instances, the subject has received two lines of prior therapy. In some instances, the subject has received three lines of prior therapy. In some instances, the subject has received four lines of prior therapy. In some instances, the subject has received more than four lines of prior therapy. In some instances, the subject experienced disease progression during or following treatment with the prior anti-cancer therapy. In some instances, the prior therapy is chemotherapy, surgery, and/or radiotherapy.

In some instances, the subject has not received prior systemic therapy (e.g., prior systemic therapy with curative intent, e.g., chemotherapy) within at least the month prior to the administration with the PD-1 axis binding antagonist and the anti-TIGIT antagonist antibody (e.g., within the two months prior, three months prior, four months prior, six months prior, one year prior, two years prior, three years prior, four years prior, five years prior, or ten years prior to the administration with the PD-1 axis binding antagonist and the anti-TIGIT antagonist antibody). In some instances, the subject is chemotherapy naïve. In some instances, the subject has not received prior immunotherapy.

iii. Lack of Treatment-Related Adverse Events

In some embodiments, the subject does not experience a treatment-related adverse event (AE) (e.g., a Grade 1, Grade 2, Grade 3, or Grade 4 treatment-related adverse event) during or following the one or more dosing cycles of tiragolumab and atezolizumab. In some embodiments, the subject experiences a treatment-related Grade 1 or Grade 2 adverse event during or following the one or more dosing cycles of tiragolumab and atezolizumab. In some embodiments, the subject does not experience a treatment-related Grade 3 or Grade 4 adverse event during or following the one or more dosing cycles of tiragolumab and atezolizumab. Treatment-related adverse events include, e.g., tiragolumab-related adverse events and/or atezolizumab-related adverse events. Adverse events are graded according to the National Cancer Institute Common Terminology Criteria for Adverse Events (NCI CTCAE), Version 4.0.

Causality of adverse events (e.g., determination of whether an adverse event is treatment-related) may be based on the following guidance:
Temporal relationship of event onset to the initiation of study drug.
Course of the event, considering especially the effects of dose reduction, discontinuation of study drug, or reintroduction of study drug (as applicable).
Known association of the event with the study drug or with similar treatments.
Known association of the event with the disease under study.
Presence of risk factors in the subject or use of concomitant medications known to increase the occurrence of the event.
Presence of non-treatment-related factors that are known to be associated with the occurrence of the event.
   In general, an adverse event may be attributed to the study drug (e.g., tiragolumab and/or atezolizumab) if there is a plausible temporal relationship between the onset of the adverse event and administration of the study drug, and the adverse event cannot be readily explained by the subject's clinical state, intercurrent illness, or concomitant therapies; and/or the adverse event follows a known pattern of response to the study drug; and/or the adverse event abates or resolves upon discontinuation of the study drug or dose reduction and, if applicable, reappears upon re-challenge.

An adverse event may be identified as non-treatment-related if evidence exists that the adverse event has an etiology other than the study drug (e.g., preexisting medical condition, underlying disease, intercurrent illness, or concomitant medication); and/or the adverse event has no plausible temporal relationship to administration of the study drug (e.g., cancer diagnosed 2 days after first dose of study drug).

Several potential risks exist for tiragolumab based on the mechanism of action, known effect of similar checkpoint inhibitors, and nonclinical data. As an antagonist of TIGIT, tiragolumab is anticipated to enhance T-cell and NK cell proliferation, survival, and function. Therefore, tiragolumab may increase the risk of autoimmune inflammation (also described as immune-mediated adverse events). In addition, due to the intact Fc-effector function of tiragolumab, lymphopenia via antibody-dependent cellular cytotoxicity (ADCC) is a theoretical risk. Particular adverse events associated with itagolumab include infusion-related reactions (IRRs), immune-mediated adverse events, and lymphopenia.

Atezolizumab has been associated with risks such as the following: IRRs and immune-mediated hepatitis, pneumonitis, colitis, pancreatitis, diabetes mellitus, hypothyroidism, hyperthyroidism, adrenal insufficiency, hypophysitis, Guillain-Barre syndrome, myasthenic syndrome or myasthenia gravis, meningoencephalitis, myocarditis, myositis and nephritis. Immune-mediated adverse reactions may involve any organ system and may lead to hemophagocytic lymphohistiocytosis (HLH) and macrophage activation syndrome (MAS).

iv. Responses to Treatment

In some embodiments of any of the methods described herein, the response to the treatment (e.g., atezolizumab and tiragolumab following an nCRT regimen) of a subject or population of subjects having a rectal cancer (e.g., a locally advanced rectal cancer (LARC)) can be characterized by one or more measures. In some embodiments, the treatment results in an increase in pCR, RFS, or EFS in the subject. In some embodiments, the treatment results in an increase in the ORR, pCR rate, RFS rate, EFS rate, or R0 resection rate in the population of subjects. In some embodiments, the treatment results in SD, a CR, or a PR in the subject.

For example, in embodiments in which an anti-TIGIT antagonist antibody (e.g., tiragolumab) in combination with a PD-1 axis binding antagonist (e.g., atezolizumab) is administered to a subject or population of subjects following a nCRT regimen, the treatment may result in pCR in the subject or population of subjects. In another example, in embodiments in which an anti-TIGIT antagonist antibody (e.g., tiragolumab) in combination with a PD-1 axis binding antagonist (e.g., atezolizumab) is administered to a population of subjects following a nCRT regimen, the treatment may result in an increase in pCR rate as compared to a reference pCR rate, e.g., as compared to nCRT not followed by treatment with a PD-1 axis binding antagonist (e.g., atezolizumab) and an anti-TIGIT antagonist antibody (e.g., tiragolumab), and/or as compared to nCRT followed by treatment with a PD-1 axis binding antagonist.

In some embodiments, a treatment described herein results in an increase in pCR rate as compared to a reference pCR rate by at least about 1% (e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%). In some embodiments, the reference pCR rate is an R0 resection rate of a population of subjects who have received a treatment comprising nCRT not followed by treatment with a PD-1 axis binding antagonist (e.g., atezolizumab) and an anti-TIGIT antagonist antibody (e.g., tiragolumab), and/or a treatment comprising nCRT followed by treatment with a PD-1 axis binding antagonist.

For example, in embodiments in which an anti-TIGIT antagonist antibody (e.g., tiragolumab) in combination with a PD-1 axis binding antagonist (e.g., atezolizumab) is administered to a population of subjects following a nCRT regimen, the treatment may result in an increase in R0 resection rate as compared to a reference R0 resection rate.

In some embodiments, a treatment described herein results in an increase in R0 resection rate as compared to a reference R0 resection by at least about 1% (e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%). In some embodiments, the reference R0 resection rate is an R0 resection rate of a population of subjects who have received a treatment comprising nCRT not followed by treatment with a PD-1 axis binding antagonist and an anti-TIGIT antagonist antibody, and/or a treatment comprising nCRT followed by treatment with a PD-1 axis binding antagonist.

For example, in embodiments in which an anti-TIGIT antagonist antibody (e.g., tiragolumab) in combination with a PD-1 axis binding antagonist (e.g., atezolizumab) is administered to a population of subjects following a nCRT regimen, the treatment may result in an increase in RFS rate as compared to a reference RFS rate.

In some embodiments, a treatment described herein results in an increase in PFS as compared to a reference PFS by at least about 1 month (e.g., 1 month, 2 months, 3.0 months, 4.0 months, 5.0 months, 6.0 months, 7.0 months, 8.0 months, 9.0 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the reference PFS is a PFS of a population of subjects who have received a treatment comprising capecitabine and oxaliplatin and not comprising a PD-1 axis binding antagonist (e.g., atezolizumab) and an anti-TIGIT antagonist antibody (e.g., tiragolumab); and/or a treatment comprising capecitabine, oxaliplatin, and a PD-1 axis binding antagonist (e.g., atezolizumab) and not comprising an anti-TIGIT antagonist antibody (e.g., tiragolumab).

In some embodiments, a treatment described herein results in an increase in OS as compared to a reference OS by at least about 1 month (e.g., 1 month, 2 months, 3.0 months, 4.0 months, 5.0 months, 6.0 months, 7.0 months, 8.0 months, 9.0 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the reference OS is an OS of a population of subjects who have received a treatment comprising capecitabine and oxaliplatin and not comprising a PD-1 axis binding antagonist (e.g., atezolizumab) and an anti-TIGIT antagonist antibody (e.g., tiragolumab); and/or a treatment comprising capecitabine, oxaliplatin, and a PD-1 axis binding antagonist (e.g., atezolizumab) and not comprising an anti-TIGIT antagonist antibody (e.g., tiragolumab).

For example, in embodiments in which an anti-TIGIT antagonist antibody (e.g., tiragolumab) in combination with a PD-1 axis binding antagonist (e.g., atezolizumab) is administered to a population of subjects following a nCRT regimen, the treatment may result in an increase in ORR of the population of subjects, e.g., as compared to a reference ORR from a population of subjects who received nCRT not followed by treatment with a PD-1 axis binding antagonist (e.g., atezolizumab) and an anti-TIGIT antagonist antibody (e.g., tiragolumab), and/or as compared to a reference ORR from a population of subjects who received nCRT followed by treatment with a PD-1 axis binding antagonist (e.g., atezolizumab). In some embodiments, a treatment described herein results in an increase in ORR as compared to a reference ORR by at least about 1% (e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%). In some embodiments, the reference ORR is an ORR of a population of subjects who have received a treatment comprising nCRT not followed by treatment with a PD-1 axis binding antagonist (e.g., atezolizumab) and an anti-TIGIT antagonist antibody (e.g., tiragolumab), and/or a treatment comprising nCRT followed by treatment with a PD-1 axis binding antagonist.

In some aspects, the clinical response is a partial response (PR).

In some aspects, the clinical response is a compete response (CR).

In some aspects, the clinical response is a reduction in the sum of diameters of one or more target lesions (e.g., rectal cancer tumors). In some aspects, the sum of diameters is decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or the sum of diameters is decreased by 100% (e.g., target lesions disappear) during or following administration of the one or more dosing cycles of an anti-TIGIT antagonist antibody (e.g., tiragolumab) and a PD-1 axis binding antagonist (e.g., atezolizumab), e.g., is decreased relative to a measurement taken before administration of the one or more dosing cycles of tiragolumab and atezolizumab.

In some aspects, the clinical response is maintained for at least 1 month, at least 2 months, at least 3 months, at least 4 months, at least 5 months, at least 6 months, at least 7 months, at least 8 months, at least 9 months, at least 10 months, at least 11 months, at least 1 year, at least 1 year and 1 month, at least 1 year and 2 months, at least 1 year and 3 months, at least 1 year and 4 months, at least 1 year and 5 months, at least 1 year and 6 months, at least 1 year and 7 months, at least 1 year and 8 months, at least 1 year and 9 months, at least 1 year and 10 months, at least 1 year and 11 months, at least 2 years, at least 2 years and 1 month, at least 2 years and 2 months, at least 2 years and 3 months, at least 2 years and 4 months, at least 2 years and 5 months, at least 2 years and 6 months, at least 2 years and 7 months, at least 2 years and 8 months, at least 2 years and 9 months, at least 2 years and 10 months, at least two years and 11 months, at least 3 years, at least 3.5 years, at least 4 years, at least 4.5 years, at least 5 years, at least 5.5 years, at least 6 years, at least 6.5 years, at least 7 years, at least 7.5 years, at least 8 years, at least 8.5 years, at least 9 years, at least 9.5 years, at least 10 years, or more than 10 years (e.g., the clinical response is maintained for 1-2 months, 2-4 months, 4-6 months, 6-8 months, 8-10 months, 10-12 months, 1 year to 1.5 years, 1.5 years to 2 years, 2 years to 2.5 years, 2.5 years to 3 years, 3 years to 3.5 years, 3.5 years to 4 years, 4 years to 4.5 years, 4.5 years-5 years, 5 years to 6 years, 6 years to 7 years, 7 years to 8 years, 8 years to 9 years, or 9 years to 10 years). For example, in some aspects, the clinical response is maintained for 1 month to 10 years, 6 months to 5 years, 1 year to 4 years, 1 year to 3 years, or 1 year to 2 years.

In some aspects, the clinical response is maintained for at least 1 year. In some aspects, the clinical response is maintained for at least 2 years.

### C. Combination Dosing of Anti-TIGIT Antagonist Antibodies and PD-1 Axis Binding Antagonists

In some instances, a dose of an effective amount of an anti-TIGIT antagonist antibody (e.g., an anti-TIGIT antagonist antibody as disclosed herein, e.g., tiragolumab) is administered with a dose of an effective amount of a PD-1 axis binding antagonist (e.g., anti-PD-L1 antagonist antibody (e.g., atezolizumab)) in a combination therapy (e.g., a combination treatment of an anti-TIGIT antagonist antibody (e.g., an anti-TIGIT antagonist antibody as disclosed herein, e.g., tiragolumab) with a PD-1 axis binding antagonist (e.g., anti-PD-L1 antagonist antibody (e.g., atezolizumab)) for treatment of a subject having a cancer (e.g., GC or GEJC (e.g., inoperable, locally advanced, metastatic, or advanced GC or GEJC) or rectal cancer (e.g., LARC)).

The present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every three weeks (e.g., on Day 1 of each 21-day dosing cycle). In some instances, the anti-TIGIT antagonist antibody (e.g., an anti-TIGIT antagonist antibody as disclosed herein, e.g., tiragolumab) is administered every three weeks (e.g., on Day 1 of each 21-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In certain instances, the present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every three weeks (e.g., on Day 1 of each 21-day dosing cycle). In some instances, the present invention includes a method of treating a subject or population of subjects having a cancer, the method comprising administering to the subject or population of subjects a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent twice daily for two weeks. In some instances, the method comprises administering to the subject or population of subjects a dosing regimen comprising one or more dosing cycles of an anti-TIGIT antagonist antibody at a dose of 500 mg to 700 mg every three weeks, a PD-1 axis binding antagonist at a dose of 900 mg to 1500 mg every three weeks, a platinum-based chemotherapeutic agent every three weeks, and a non-platinum-based chemotherapeutic agent twice daily every two weeks.

In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from about 0.01 mg/kg to about 50 mg/kg (e.g., about 15 mg/kg) up to 1200 mg, e.g., every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from 0.01 mg/kg to 50 mg/kg (e.g., 15 mg/kg) up to 1200 mg, e.g., every three weeks. Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biostimulation studies based on extrapolations of pharmacokinetic parameters estimated from adult data. In some instances, the dose (e.g., about 600 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW)>40 kg: 600 mg, BW>15 kg and ≤40 kg: 400 mg, and BW≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW)>40 kg: 600 mg, BW>15 kg and ≤40 kg: 400 mg, and BW≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body surface area (e.g., BSA>1.25 m²: 600 mg, BSA>0.75 m² and ≤1.25 m²: 450 mg, BSA>0.5 m² and ≤0.75 m²: 350 mg, and BSA≤0.5 m²: 300 mg) every three weeks. In some embodiments, the PD-1 axis binding antagonist (e.g., anti-PD-L1 antagonist antibody (e.g., atezolizumab)) is administered at a maximum dose of 1200 mg every three weeks.

i. Dosing of Anti-TIGIT Antagonist Antibodies

As a general proposition, the therapeutically effective amount of an anti-TIGIT antagonist antibody (e.g., an anti-TIGIT antagonist antibody as disclosed herein, e.g., tiragolumab) administered to a subject having a cancer (e.g., GC or GEJC (e.g., inoperable, locally advanced, metastatic, or advanced GC or GEJC) or rectal cancer (e.g., LARC)) will be in the range of about 0.01 to about 50 mg/kg of subject body weight, whether by one or more administrations. In some embodiments, the therapeutically effective amount of an anti-TIGIT antagonist antibody (e.g., an anti-TIGIT antagonist antibody as disclosed herein, e.g., tiragolumab) administered to a subject is in the range of 0.01 to 50 mg/kg of subject body weight, whether by one or more administrations.

In some exemplary embodiments, the anti-TIGIT antagonist antibody (e.g., an anti-TIGIT antagonist antibody as disclosed herein, e.g., tiragolumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In exemplary embodiments, the anti-TIGIT antagonist antibody (e.g., an anti-TIGIT antagonist antibody as disclosed herein, e.g., tiragolumab) is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the anti-TIGIT antagonist antibody (e.g., an anti-TIGIT antagonist antibody as disclosed herein, e.g., tiragolumab) is administered on about Day 1 (e.g., Day −3, Day −2, Day −1, Day 1, Day 2, or Day 3) of a dosing cycle.

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W). In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab)) may be reduced as compared to a standard dose of the anti-TIGIT antagonist antibody administered as a monotherapy.

In some instances, the effective amount of the anti-TIGIT antagonist antibody (e.g., an anti-TIGIT antagonist antibody as disclosed herein, e.g., tiragolumab) is a fixed dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a fixed dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks).

In some instances, the effective amount of the anti-TIGIT antagonist antibody (e.g., an anti-TIGIT antagonist antibody as disclosed herein, e.g., tiragolumab) is a fixed dose of between about 200 mg to about 2000 mg (e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every four weeks (Q4W). In some instances, the effective amount of anti-TIGIT antagonist antibody (e.g., an anti-TIGIT antagonist antibody as disclosed herein, e.g., tiragolumab) is a fixed dose of about 840 mg every four weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every four weeks).

In some instances, the dose of the anti-TIGIT antagonist antibody (e.g., an anti-TIGIT antagonist antibody as disclosed herein, e.g., tiragolumab) is a tiered dose based on a subject's body weight (e.g., body weight (BW)>40 kg: 600 mg, BW>15 kg and ≤40 kg: 400 mg, and BW≤15 kg: 300 mg).

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)) may be reduced as compared to a standard dose of the anti-TIGIT antagonist antibody administered as a monotherapy.

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, tiragolumab is administered to the subject intravenously at a dose of about 420 mg every 2 weeks, about 600 mg every 3 weeks, or about 840 mg of every 4 weeks. In some instances, tiragolumab is administered to the subject intravenously at a dose of 420 mg every 2 weeks, 600 mg every 3 weeks, or 840 mg of every 4 weeks.

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody administered as a monotherapy. In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel or nab-paclitaxel), and/or a topoisomerase II inhibitor (e.g., doxorubicin))) and/or G-CSF or GM-CSF, may be reduced as compared to a standard dose of the anti-TIGIT antagonist antibody administered as a monotherapy.

In some instances, a subject is administered a total of 1 to 60 doses of an anti-TIGIT antagonist antibody (e.g., an anti-TIGIT antagonist antibody as disclosed herein, e.g., tiragolumab), e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 doses. In some instances, a subject is administered a total of 1 to 60 doses of an anti-TIGIT antagonist antibody (e.g., an anti-TIGIT antagonist antibody as disclosed herein, e.g., tiragolumab), e.g., 1 to 60 doses, 1 to 55 doses, 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 60 doses, 2 to 55 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 60 doses, 3 to 55 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 60 doses, 4 to 55 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 60 doses, 5 to 55 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 60 doses, 10 to 55 doses, 10 to 50 doses, 10 to 45 doses, 10 to 40 doses, 10 to 35 doses, 10 to 30 doses, 10 to 25 doses, 10 to 20 doses, 10 to 15 doses, 15 to 60 doses, 15 to 55 doses, 15 to 50 doses, 15 to 45 doses, 15 to 40 doses, 15 to 35 doses, 15 to 30 doses, 15 to 25 doses, 15 to 20 doses, 20 to 60 doses, 20 to 55 doses, 20 to 50 doses, 20 to 45 doses, 20 to 40 doses, 20 to 35 doses, 20 to 30 doses, 20 to 25 doses, 25 to 60 doses, 25 to 55 doses, 25 to 50 doses, 25 to 45 doses, 25 to 40 doses, 25 to 35 doses, 25 to 30 doses, 30 to 60 doses, 30 to 55 doses, 30 to 50 doses, 30 to 45 doses, 30 to 40 doses, 30 to 35 doses, 35 to 60 doses, 35 to 55 doses, 35 to 50 doses, 35 to 45 doses, 35 to 40 doses, 40 to 60 doses, 40 to 55 doses, 40 to 50 doses, 40 to 45 doses, 45 to 50 doses, 50 to 60 doses, or 55 to 60 doses. In particular instances, the doses may be administered intravenously.

In some instances, atezolizumab is administered to the subject intravenously at a dose of about 840 mg every 2 weeks, about 1200 mg every 3 weeks, or about 1680 mg of every 4 weeks.

The PD-1 axis binding antagonist and anti-TIGIT antagonist antibody may be administered in any suitable manner known in the art. For example, the PD-1 axis binding antagonist and anti-TIGIT antagonist antibody may be administered sequentially (on different days) or concurrently (on the same day or during the same treatment cycle). In some instances, the anti-TIGIT antagonist antibody and/or the PD-1 axis binding antagonist are administered on about Day 1 (e.g., Day −3, Day −2, Day −1, Day 1, Day 2, or Day 3) of a dosing cycle. In some instances, the PD-1 axis binding antagonist and anti-TIGIT antagonist antibody may be administered on the same day. In some instances, the PD-1 axis binding antagonist is administered before the anti-TIGIT antagonist antibody. In some instances, the PD-1 axis binding antagonist is administered after the anti-TIGIT antagonist antibody. In some instances, the PD-1 axis binding antagonist is administered simultaneously with the anti-TIGIT antagonist antibody. In some instances, the PD-1 axis binding antagonist may be administered prior to an anti-TIGIT antagonist antibody that is administered on the same day. In some instances, the PD-1 axis binding antagonist may be administered after to an anti-TIGIT antagonist antibody that is administered on the same day. In yet other instances, the PD-1 axis binding antagonist is administered at the same time as the anti-TIGIT antagonist antibody. In some instances, the PD-1 axis binding antagonist is in a separate composition as the anti-TIGIT antagonist antibody. In some instances, the PD-1 axis binding antagonist is in the same composition as the anti-TIGIT antagonist antibody. In some instances, the PD-1 axis binding antagonist is administered through a separate intravenous line from any other therapeutic agent administered to the subject on the same day. The PD-1 axis binding antagonist and anti-TIGIT antagonist antibody may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the PD-1 axis binding antagonist is administered intravenously. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously. In some instances, there is a first observation period following administration of the PD-1 axis binding antagonist. In some instances, there is a second observation period following administration of the PD-1 axis binding antagonist. In some instances, there is a first observation period following administration of the anti-TIGIT antagonist antibody. In some instances, there is a second observation period following administration of the anti-TIGIT antagonist antibody. In some instances, the observation period is between about 30 minutes to about 60 minutes in length. In some instances, the anti-TIGIT antagonist antibody and/or PD-1 axis binding antagonist are administered intravenously or subcutaneously.

In some instances, the intravenous infusion is over 30±10 minutes and/or over 60±10 minutes. In one example, atezolizumab may be administered intravenously over 60 minutes; if the first infusion is tolerated, all subsequent infusions may be delivered over 30 minutes. In one example, tiragolumab may be administered intravenously over 60 minutes; if the first infusion is tolerated, all subsequent infusions may be delivered over 30 minutes.

In some examples, the PD-1 axis binding antagonist is not administered as an intravenous push or bolus. In some examples, the anti-TIGIT antagonist antibody is not administered as an intravenous push or bolus.

In any of the preceding examples, each dosing cycle may have any suitable length, e.g., about 7 days (about 5, 6, 7, 8, or 9 days), about 14 days (e.g., about 12, 13, 14, 15, or 16 days), about 21 days (e.g., about 18, 19, 20, 21, 22, 23, or 24 days), about 28 days (about 25, 26, 27, 28, 29, 30, or 31 days), or longer. In some instances, each dosing cycle is about 21 days.

ii. Dosing of PD-1 Axis Binding Antagonists

As a general proposition, the therapeutically effective amount of a PD-1 axis binding antagonist (e.g., atezolizumab) administered to a subject having a cancer (e.g., GC or GEJC (e.g., inoperable, locally advanced, metastatic, or advanced GC or GEJC) or rectal cancer (e.g., LARC)) will be in the range of about 0.01 to about 50 mg/kg of subject body weight, whether by one or more administrations.

In some exemplary embodiments, the PD-1 axis binding antagonist (e.g., atezolizumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In some instances, the dose of the PD-1 axis binding antagonist (e.g., anti-PD-L1 antagonist antibody (e.g., atezolizumab)) is a dose based on a subject's body weight (e.g., 15 mg/kg). In some instances, the dose of the PD-1 axis binding antagonist (e.g., anti-PD-L1 antagonist antibody (e.g., atezolizumab)) is a dose based on a subject's body surface area (e.g., body surface area (BSA)>1.25 m²: 600 mg, BSA>0.75 m² and ≤1.25 m²: 450 mg, BSA>0.5 m² and ≤0.75 m²: 350 mg, and BSA≤0.5 m²: 300 mg).

In some instances, the effective amount of the PD-1 axis binding antagonist (e.g., an anti-PD-L1 antagonist antibody (e.g., atezolizumab)) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some embodiments, the effective amount of the PD-1 axis binding antagonist is atezolizumab at a fixed dose of about 1200 mg every three weeks. In some embodiments, the effective amount of the PD-1 axis binding antagonist is pembrolizumab at a fixed dose of about 200 mg every three weeks or, alternatively, pembrolizumab at a fixed dose of about 400 mg every six weeks.

In some instances, the fixed dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., an anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., an anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of PD-1 axis binding antagonist (e.g., an anti-PD-L1 antagonist antibody (e.g., atezolizumab)) is a dose of about 15 mg/kg administered every three weeks. In some instances, the dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., anti-PD-L1 antagonist antibody (e.g., atezolizumab)) is a fixed dose of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist is atezolizumab at a fixed dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some embodiments, the effective amount of the PD-1 axis binding antagonist is avelumab at a fixed dose of about 800 mg every two weeks. In some embodiments, the effective amount of the PD-1 axis binding antagonist is nivolumab at a fixed dose of about 240 mg every two weeks.

In some instances, the effective amount of the PD-1 axis binding antagonist (e.g., anti-PD-L1 antagonist antibody (e.g., atezolizumab)) is a fixed dose of between about 500 mg to about 3000 mg (e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab)) is a fixed dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680±0.5 mg, e.g., 1680 mg every four weeks). In some embodiments, the effective amount of the PD-1 axis binding antagonist is nivolumab at a fixed dose of about 480 mg every four weeks.

In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein (e.g., tiragolumab) may be reduced as compared to a standard dose of the anti-PD-L1 antagonist antibody administered as a monotherapy. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF may be reduced as compared to a standard dose of the PD-1 axis binding antagonist administered as a monotherapy.

In some instances, a subject is administered a total of 1 to 60 doses of a PD-1 axis binding antagonist (e.g., atezolizumab), e.g., 1 to 60 doses, 1 to 55 doses, 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 60 doses, 2 to 55 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 60 doses, 3 to 55 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 60 doses, 4 to 55 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 60 doses, 5 to 55 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 60 doses, 10 to 55 doses, 10 to 50 doses, 10 to 45 doses, 10 to 40 doses, 10 to 35 doses, 10 to 30 doses, 10 to 25 doses, 10 to 20 doses, 10 to 15 doses, 15 to 60 doses, 15 to 55 doses, 15 to 50 doses, 15 to 45 doses, 15 to 40 doses, 15 to 35 doses, 15 to 30 doses, 15 to 25 doses, 15 to 20 doses, 20 to 60 doses, 20 to 55 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 60 doses, 30 to 55 doses, 30 to 50 doses, 30 to 45 doses, 30 to 40 doses, 30 to 35 doses, 35 to 60 doses, 35 to 55 doses, 35 to 50 doses, 35 to 45 doses, 35 to 40 doses, 40 to 60 doses, 40 to 55 doses, 40 to 50 doses, 40 to 45 doses, 45 to 50 doses, 50 to 60 doses, or 55 to 60 doses. In particular instances, the doses may be administered intravenously.

In some instances, atezolizumab is administered to the subject intravenously at a dose of about 840 mg every 2 weeks, about 1200 mg every 3 weeks, or about 1680 mg of every 4 weeks. For example, in some aspects, atezolizumab is administered to the subject intravenously at a dose of 1200 mg every 3 weeks. In some aspects, atezolizumab is administered to the subject intravenously at a dose of 840 mg every 2 weeks. In some aspects, atezolizumab is administered to the subject intravenously at a dose of 1680 mg every 4 weeks.

The PD-1 axis binding antagonist and/or any additional therapeutic agent(s) may be administered in any suitable manner known in the art. For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the subject on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating a cancer (e.g., GC or GEJC (e.g., inoperable, locally advanced, metastatic, or advanced GC or GEJC) or rectal cancer (e.g., LARC)) in a subject comprising administering to the subject a treatment regimen comprising effective amounts of a PD-1 axis binding antagonist (e.g., atezolizumab) and/or an anti-TIGIT antagonist antibody (e.g., tiragolumab) in combination with another anti-cancer agent or cancer therapy (e.g., capecitabine and oxaliplatin). For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with capecitabine, oxaliplatin, bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

iii. Dosing Cycles for Anti-TIGIT Antagonist Antibodies and PD-1 Axis Binding Antagonists

In any of the methods and uses of the invention, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) may be administered to the subject having a cancer (e.g., GC or GEJC (e.g., inoperable, locally advanced, metastatic, or advanced GC or GEJC) or rectal cancer (e.g., LARC)) in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more dosing cycles). In some aspects, the one or more dosing cycles comprise administration of one or more doses of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) as described in Sections B(i) and B(ii), respectively, to the subject having a cancer (e.g., GC or GEJC (e.g., inoperable, locally advanced, metastatic, or advanced GC or GEJC) or rectal cancer (e.g., LARC)). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) continue until there is a loss of clinical benefit (e.g., confirmed disease progression, drug resistance, death, or unacceptable toxicity). In some instances, the length of each dosing cycle is about 7 to 42 days (e.g., 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 41 days, 42 days). In some instances, the length of each dosing cycle is about 14 days. In some instances, the length of each dosing cycle is about 21 days. In some instances, the length of each dosing cycle is about 28 days. In some instances, the length of each dosing cycle is about 42 days. In some instances, the length of each dosing cycle is about 7 days.

In some instances, the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are administered on about Day 1 (e.g., Day 1±3 days) of each dosing cycle. In some instances, the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are administered on about Day 15 (e.g., Day 15±3 days) of each dosing cycle.

In some instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered on about Day 1 (e.g., Day 1±3 days) of each dosing cycle.

In some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks) and the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks) and the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In some instances, the anti-TIGIT antagonist antibody (e.g., an anti-TIGIT antagonist antibody as disclosed herein, e.g., tiragolumab) is administered intravenously at a dose of about 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of about 840 mg on Day 1 of each 14-day cycle (i.e., at a dose of about 840 mg every two weeks).

In some instances, the anti-TIGIT antagonist antibody (e.g., an anti-TIGIT antagonist antibody as disclosed herein, e.g., tiragolumab) is administered intravenously at a dose of 840 mg on Day 1 of each 28-day cycle (i.e., at a dose of 840 mg every four weeks) and the PD-1 axis binding antagonist (e.g., anti-PD-L1 antagonist antibody (e.g., atezolizumab)) is administered intravenously at a dose of 1680 mg on Day 1 of each 28-day cycle (i.e., at a dose of 1680 mg every four weeks).

iv. Intravenous Infusion and Subcutaneous Administration of Anti-TIGIT Antagonist Antibodies and PD-1 Axis Binding Antagonists

In some instances, the anti-TIGIT antagonist antibody (e.g., an anti-TIGIT antagonist antibody as disclosed herein, e.g., tiragolumab) is administered intravenously to the subject having a cancer (e.g., GC or GEJC (e.g., inoperable, locally advanced, metastatic, or advanced GC or GEJC) or rectal cancer (e.g., LARC)). Alternatively, in some embodiments, the anti-TIGIT antagonist antibody (e.g., an anti-TIGIT antagonist antibody as disclosed herein, e.g., tiragolumab) is administered subcutaneously. In some instances, the PD-1 axis binding antagonist (e.g., anti-PD-L1 antagonist antibody (e.g., atezolizumab)) is administered intravenously. Alternatively, in some embodiments, the PD-1 axis binding antagonist (e.g., anti-PD-L1 antagonist antibody (e.g., atezolizumab)) is administered subcutaneously.

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject or population of subjects by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab) is administered to the subject by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the PD-1 axis binding antagonist (e.g., anti-PD-L1 antagonist antibody (e.g., atezolizumab)) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

v. Administration Order and Observation Periods

In some instances in which both an anti-TIGIT antagonist antibody and PD-1 axis binding antagonist are administered to a subject or population of subjects having cancer (e.g., GC or GEJC (e.g., inoperable, locally advanced, metastatic, or advanced GC or GEJC) or rectal cancer (e.g., LARC)), the anti-TIGIT antagonist antibody (e.g., an anti-TIGIT antagonist antibody as disclosed herein, e.g., tiragolumab) is administered to the subject before the PD-1 axis binding antagonist (e.g., anti-PD-L1 antagonist antibody (e.g., atezolizumab)).

In some instances, for example, following administration of the anti-TIGIT antagonist antibody and before administration of the PD-1 axis binding antagonist the method includes an intervening first observation period. In some instances, for example, following administration of the anti-TIGIT antagonist antibody, the PD-1 axis binding antagonist (e.g., anti-PD-L1 antagonist antibody (e.g., atezolizumab)) is administered to the subject. In some instances, the anti-TIGIT antagonist antibody (e.g., an anti-TIGIT antagonist antibody as disclosed herein, e.g., tiragolumab) is first administered to the subject and the PD-1 axis binding antagonist (e.g., anti-PD-L1 antagonist antibody (e.g., atezolizumab)) is administered to the subject following administration of the anti-TIGIT antagonist antibody (e.g., an anti-TIGIT antagonist antibody as disclosed herein, e.g., tiragolumab).

In some instances, the method further includes a second observation period following administration of the PD-1 axis binding antagonist (e.g., anti-PD-L1 antagonist antibody (e.g., atezolizumab)).

In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab)). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody, the PD-1 axis binding antagonist (e.g., anti-PD-L1 antagonist antibody (e.g., atezolizumab)) during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody or the PD-1 axis binding antagonist (e.g., anti-PD-L1 antagonist antibody (e.g., atezolizumab)) during the first or second.

In some instances, the PD-1 axis binding antagonist (e.g., anti-PD-L1 antagonist antibody (e.g., atezolizumab)) is administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab)) and before administration of the anti-TIGIT antagonist antibody (e.g., an anti-TIGIT antagonist antibody as disclosed herein, e.g., tiragolumab), the method includes an intervening first observation period.

In some instances, the method further includes a second observation period following administration of the anti-TIGIT antagonist antibody (e.g., an anti-TIGIT antagonist antibody as disclosed herein, e.g., tiragolumab).

In some instances, the method includes both a first observation period following administration of the PD-1 axis binding antagonist (e.g., anti-PD-L1 antagonist antibody (e.g., atezolizumab)) and second observation period following administration of the anti-TIGIT antagonist antibody (e.g., an anti-TIGIT antagonist antibody as disclosed herein, e.g., tiragolumab). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab)) or the anti-TIGIT antagonist antibody (e.g., an anti-TIGIT antagonist antibody as disclosed herein, e.g., tiragolumab), during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), during the first or second observation periods.

### III. Assessment of PD-L1 Expression

The expression of PD-L1 may be assessed in a subject treated according to any of the methods and compositions for use described herein. The methods and compositions for use may include determining the expression level of PD-L1 in a biological sample (e.g., a tumor sample) obtained from the subject having a cancer (e.g., GC or GEJC (e.g., inoperable, locally advanced, metastatic, or advanced GC or GEJC) or rectal cancer (e.g., LARC)). In other examples, the expression level of PD-L1 in a biological sample (e.g., a tumor sample) obtained from the subject has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the subject has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the subject has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some aspects, the GC or GEJC (e.g., inoperable, locally advanced, metastatic, or advanced GC or GEJC) or rectal cancer (e.g., LARC) of a subject treated according to any of the methods provided herein has a PD-L1-positive tumor cell (TC) fraction or tumor-infiltrating immune cell (IC) fraction of <5%. In some aspects, the GC, GEJC, or rectal cancer has a PD-L1-positive TC fraction of <1%. In other aspects, the GC, GEJC, or rectal cancer of a subject treated according to any of the methods provided herein has a PD-L1-positive TC fraction or IC fraction of ≥ 5%. In some aspects, PD-L1 is detected using a Ventana SP142 IHC assay, a Ventana SP263 IHC assay, a pharmDx 22C3 IHC assay, or a pharmDx 28-8 IHC assay.

In some examples, tumor samples may be scored for PD-L1 positivity in tumor-infiltrating immune cells and/or in tumor cells according to the criteria for diagnostic assessment shown in Table A and/or Table B, respectively.

TABLE A
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor cells, | |
| associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by tumor | |
| cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor cells, | |
| associated intratumoral stroma, and contiguous | |
| peri-tumoral desmoplastic stroma | |

TABLE B
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### IV. Assessment of TIGIT Expression

The expression level of TIGIT may be assessed in a subject having a cancer (e.g., GC or GEJC (e.g., inoperable, locally advanced, metastatic, or advanced GC or GEJC) or rectal cancer (e.g., LARC)) who has been treated according to any of the methods, uses, and compositions for use described herein. The methods, uses, and compositions for use may include determining the expression level of TIGIT in a biological sample (e.g., a tumor sample) obtained from the subject. In other examples, the expression level of TIGIT in a biological sample (e.g., a tumor sample) obtained from the subject has been determined prior to initiation of treatment or after initiation of treatment. TIGIT expression may be determined using any suitable approach. Any suitable tumor sample may be used, e.g., a formalin-fixed and paraffin-embedded (FFPE) tumor sample, an archival tumor sample, a fresh tumor sample, or a frozen tumor sample.

For example, TIGIT expression may be determined in terms of the percentage of a tumor sample comprised by tumor-infiltrating immune cells expressing a detectable expression level of TIGIT, as the percentage of tumor-infiltrating immune cells in a tumor sample expressing a detectable expression level of TIGIT, and/or as the percentage of tumor cells in a tumor sample expressing a detectable expression level of TIGIT. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-TIGIT antagonist antibody. Any suitable anti-TIGIT antagonist antibody may be used. In some examples, the anti-TIGIT antagonist antibody is 10A7 (WO 2009/126688A3; U.S. Pat. No. 9,499,596).

### V. Anti-TIGIT Antagonist Antibodies

The invention provides anti-TIGIT antagonist antibodies useful for treating cancer in a subject (e.g., a human) having a cancer (e.g., GC or GEJC (e.g., inoperable, locally advanced, metastatic, or advanced GC or GEJC) or rectal cancer (e.g., LARC)).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14), (e) an HVR-L2 comprising the amino acid sequence of WASTRES (SEQ ID NO: 15); and/or (f) an HVR-L3 comprising the amino acid sequence of QQYYSTPFT (SEQ ID NO: 16), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 11-16.

In some instances, anti-TIGIT antagonist antibodies may include (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14); (e) an HVR-L2 comprising the amino acid sequence of WASTRES (SEQ ID NO: 15); and (f) an HVR-L3 comprising the amino acid sequence of QQYYSTPFT (SEQ ID NO: 16). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 27) or an amino acid sequence having at least 90% sequence identity (e.g., at least 91%, 92%, 93%, 94%, 95%, 96%, 97%, 98%, or 99% sequence identity) to, or the sequence of, QVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 28); and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIK (SEQ ID NO: 29). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 27 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 29. In some instances, the anti-TIGIT antagonist antibody has a VH domain comprising the amino acid sequence of SEQ ID NO: 27 and a VL domain comprising the amino acid sequence of SEQ ID NO: 29. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 28 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 29. In some instances, the anti-TIGIT antagonist antibody has a VH domain comprising the amino acid sequence of SEQ ID NO: 28 and a VL domain comprising the amino acid sequence of SEQ ID NO: 29.

In some instances, the anti-TIGIT antagonist antibody includes a heavy chain and a light chain sequence, wherein:

(a) the heavy chain comprises the amino acid sequence:
(SEQ ID NO: 33)
EVQLQQSGPGLVKPSQTLSLTCAISGDSVSSNSAAWNWIRQSPSRGLEWLGKTYYRFKWYSDYAVSVK
GRITINPDTSKNQFSLQLNSVTPEDTAVFYCTRESTTYDLLAGPFDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK;
and
(b) the light chain comprises the amino acid sequence:
(SEQ ID NO: 34)
DIVMTQSPDSLAVSLGERATINCKSSQTVLYSSNNKKYLAWYQQKPGQPPNLLIYWASTRESGVPDRFS
GSGSGTDFTLTISSLQAEDVAVYYCQQYYSTPFTFGPGTKVEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSS
PVTKSFNRGEC.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 17); an FR-L2 comprising the amino acid sequence of WYQQKPGQPPNLLIY (SEQ ID NO: 18); an FR-L3 comprising the amino acid sequence of GVPDRFSGSGSGTDFTLTISSLQAEDVAVYYC (SEQ ID NO: 19); and/or an FR-L4 comprising the amino acid sequence of FGPGTKVEIK (SEQ ID NO: 20), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 17-20. In some instances, for example, the antibody further comprises an FR-L1 comprising the amino acid sequence of DIVMTQSPDSLAVSLGERATINC (SEQ ID NO: 17); an FR-L2 comprising the amino acid sequence of WYQQKPGQPPNLLIY (SEQ ID NO: 18); an FR-L3 comprising the amino acid sequence of GVPDRFSGSGSGTDFTLTISSLQAEDVAVYYC (SEQ ID NO: 19); and an FR-L4 comprising the amino acid sequence of FGPGTKVEIK (SEQ ID NO: 20).

In some instances, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 21), wherein X₁ is E or Q; an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 21-24. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 25); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 22-25. In some instances, the anti-TIGIT antagonist antibody includes an FR-H1 comprising the amino acid sequence of EVQLQQSGPGLVKPSQTLSLTCAISGDSVS (SEQ ID NO: 25); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and an FR-H4 comprising the amino acid sequence of WGQGTLVTVSS (SEQ ID NO: 24). In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 26); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 22-24 and 26. In some instances, the anti-TIGIT antagonist antibody includes an FR-H1 comprising the amino acid sequence of QVQLQQSGPGLVKPSQTLSLTCAISGDSVS (SEQ ID NO: 26); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and an FR-H4 comprising the amino acid sequence of WGQGTLVTVSS (SEQ ID NO: 24).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a KD of about 10 nM or lower and cyno TIGIT with a KD of about 10 nM or lower (e.g., binds human TIGIT with a KD of about 0.1 nM to about 1 nM and cyno TIGIT with a KD of about 0.5 nM to about 1 nM, e.g., binds human TIGIT with a KD of about 0.1 nM or lower and cyno TIGIT with a KD of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization.

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14), (e) an HVR-L2 comprising the amino acid sequence of WASTRES (SEQ ID NO: 15); and (f) an HVR-L3 comprising the amino acid sequence of QQYYSTPFT (SEQ ID NO: 16). The methods described herein may also include administering an isolated anti-TIGIT antagonist antibody that binds to the same epitope as an anti-TIGIT antagonist antibody described above.

In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or RO7092284), vibostolimab (MK-7684), ASP8374 (PTZ-201), EOS884448 (EOS-448), SEA-TGT (SGN-TGT)), BGB-A1217, BMS-986207 (ONO-4686), COM902 (CGEN-15137), IBI939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or RO7092284), vibostolimab (MK-7684), ASP8374 (PTZ-201), EOS-448, and SEA-TGT (SGN-TGT). The anti-TIGIT antibody may be tiragolumab (MTIG7192A, RG6058 or RO7092284).

Non-limiting examples of anti-TIGIT antibodies that are useful for the methods disclosed herein, and methods for making thereof are described in PCT Pub. Nos. WO2018183889A1, WO2019129261A1, WO2016106302A9, WO2018033798A1, WO2020020281A1, WO2019023504A1, WO2017152088A1, WO2016028656A1, WO2017030823A2, WO2018204405A1, WO2019152574A1, and WO2020041541A2; U.S. Pat. Nos. 10,189,902, 10,213,505, 10,124,061, 10,537,633, and 10,618,958; and U.S. Pub. Nos. 2020/0095324, 2019/0112375, 2018/0371083, and 2020/0062859, each of which is incorporated herein by reference in its entirety. Additional non-limiting examples of anti-TIGIT antibodies, useful for the methods of disclosed herein, and methods for making thereof are described in PCT Pub. Nos. WO2018204363A1, WO2018047139A1, WO2019175799A2, WO2018022946A1, WO2015143343A2, WO2018218056A1, WO2019232484A1, WO2019079777A1, WO2018128939A1, WO2017196867A1, WO2019154415A1, WO2019062832A1, WO2018234793A3, WO2018102536A1, WO2019137548A1, WO2019129221A1, WO2018102746A1, WO2018160704A9, WO2020041541A2, WO2019094637A9, WO2017037707A1, WO2019168382A1, WO2006124667A3, WO2017021526A1, WO2017184619A2, WO2017048824A1, WO2019032619A9, WO2018157162A1, WO2020176718A1, WO2020047329A1, WO2020047329A1, WO2018220446A9; U.S. Pat. Nos. 9,617,338, 9,567,399, 10,604,576, and 9,994,637; and Pub. Nos. US 2018/0355040, US 2019/0175654, US 2019/0040154, US 2019/0382477, US 2019/0010246, US 2020/0164071, US 2020/0131267, US 2019/0338032, US 2019/0330351, US 2019/0202917, US 2019/0284269, US 2018/0155422, US 2020/0040082, US 2019/0263909, US 2018/0185480, US 2019/0375843, US 2017/0037133, US 2019/0077869, US 2019/0367579, US 2020/0222503, US 2020/0283496, CN109734806A, and CN110818795A, each of which is incorporated herein by reference in its entirety.

The anti-TIGIT antibodies useful in the methods disclosed herein include ASP8374 (PTZ-201), BGB-A1217, BMS-986207 (ONO-4686), COM902 (CGEN-15137), M6223, IBI939, EOS-448, domvanalimab (AB154), vibostolimab (MK-7684), and SEA-TGT (SGN-TGT). Additional anti-TIGIT antibodies useful in the methods disclosed herein include AGEN1307; AGEN1777; antibody clones pab2197 and pab2196 (Agenus Inc.); antibody clones TBB8, TDC8, 3TB3, 5TB10, and D1Y1A (Anhui Anke Biotechnology Group Co. Ltd.), antibody clones MAB1, MAB2, MAB3, MAB4, MAB5, MAB6, MAB 7, MAB8, MAB9, MAB 10, MAB 11, MAB 12, MAB13, MAB 14, MAB 15, MAB 16, MAB 17, MAB 18, MAB19, MAB20, MAB21 (Astellas Pharma/Potenza Therapeutics), antibody clones hu1217-1-1 and hu1217-2-2 (BeiGene), antibody clones 4D4 and 19G (Brigham & Women's Hospital), antibody clones 11G11, 10D7, 15A6, 22G2, TIGIT G2a, and TIGIT G1 D265A, including such antibodies with modified heavy chain constant regions (Bristol-Myers Squibb); antibody clones 10A7, CPA.9.086, CPA.9.083.H4(S241P), CPA.9.086.H4(S241P), CHA.9.547.7.H4(S241P) and CHA.9.547.13.H4(S241P) (Compugen); anti-PVRIG/anti-TIGIT bispecific antibodies (Compugen), antibody clones 315293, 328189, 350426, 326504, and 331672 (Fred Hutchinson Cancer Research Center); antibody clones T-01, T-02, T-03, T-04, T-05, T-06, T-07, T-08, T-09, and T-10 (Gensun BioPharma Inc.); antibody clones 1H6, 2B11, 3A10, 4A5, 4A9, 4H5, 6A2, 6B7, 7F4, 8E1, 8G3, 9F4, 9G6, 10C1, 10F10, 11G4, 12B7, 12C8, 15E9, 16C11, 16D6, and 16E10 (Hefei Ruida Immunological Drugs Research Institute Co. Ltd.); antibody clones h3C5H1, h3C5H2, h3C5H3, h3C5H4, h3C5H3-1, h3C5H3-2, h3C5H3-3, h3C5L1, and h3C5L2 (IGM Biosciences Inc.); antibody clones 90D9, 101E1, 116H8, 118A12, 131A12, 143B6, 167F7, 221F11, 222H4, 327C9, 342A9, 344F2, 349H6, and 350D10 (I-Mab Biopharma); antibody clones ADI-27238, ADI-30263, ADI-30267, ADI-30268, ADI-27243, ADI-30302, ADI-30336, ADI-27278, ADI-30193, ADI-30296, ADI-27291, ADI-30283, ADI-30286, ADI-30288, ADI27297, ADI-30272, ADI-30278, ADI-27301, ADI-30306, and ADI-30311 (Innovent Biologics, Inc.); antibody clones 26518, 29478, 26452, 29487, 29489, 31282, 26486, 29494, 29499, 26521, 29513, 26493, 29520, 29523, 29527, 31288, 32919, 32931, 26432, and 32959 (iTeos Therapeutics); antibody clones m1707, m1708, m1709, m1710, m1711, h1707, h1708, h1709, h1710, and h1711 (Jiangsu Hengrui Medicine Co. Ltd.); antibody clones TIG1, TIG2, and TIG3 (JN Biosciences LLC); antibody clones (e.g., KY01, KY02, KY03, KY04, KY05, KY06, KY07, KY08, KY09, KY10, K11, K12, K13, K14, K15, K16, K17, K18, K19, K20, K21, K22, K23 Kymab TIGIT (Antibody 2), and Tool TIGIT (Antibody 4) (Kymab Limited); bispecific antibodies 1D05/in-house anti-TIGIT with 1D05 (anti-PD-L1) Native variable domain and Kymab TIGIT antigen binding site (ABS) domain (Bispecific 1), In-house anti-TIGIT/1D05 with Kymab TIGIT Native variable domain and 1D05 ABS domain (Bispecific 2), Tool anti-TIGIT/Tool anti-PD-L1 with Toon anti-TIGIT Native variable domain and Tool anti-PD-L1 ABS domain (Bispecific 3), Tool anti-PD-L1/Tool anti-TIGIT with Tool anti-PD-L1 Native variable domain and Tool anti-TIGIT ABS domain (Bispecific 4) (Kymab Limited); antibody clones and clone variants 14D7, 26B10, Hu14D7, Hu26B10, 14A6, Hu14A6, 28H5, 31C6, Hu31C6, 25G10, MBS43, 37D10, 18G10, 11A11, c18G10, and LB155.14A6.G2.A8 (Merck); etigilimab (OMP-313M32) (Mereo BioPharma); antibody clones 64G1E9B4, 100C4E7D11, 83G5H11C12, 92E9D4B4, 104G12E12G2, 121C2F10B5, 128E3F10F3F2, 70A11A8E6, 11D8E124A, 16F10H12C11, 8F2D8E7, 48B5G4E12, 139E2C2D2, 128E3G7F5, AS19584, AS19852, AS19858, AS19886, AS19887, AS19888, AS20160, AS19584VH26, AS19584VH29, AS19584VH30, AS19584VH31, AS19886VH5, AS19886VH8, AS19886VH9, AS19886VH10, AS19886VH19, AS19886VH20, AS19584VH28-Fc, AS19886VH5-Fc, AS19886VH8-Fc, AS19584-Fc, and AS19886-Fc (Nanjing Legend Biotechnology Co. Ltd.); antibody clones ARE clones: Ab58, Ab69, Ab75, Ab133, Ab177, Ab122, Ab86, Ab180, Ab83, Ab26, Ab20, Ab147, Ab12, Ab66, Ab176, Ab96, Ab123, Ab109, Ab149, Ab34, Ab61, Ab64, Ab105, Ab108, Ab178, Ab166, Ab29, Ab135, Ab171, Ab194, Ab184, Ab164, Ab183, Ab158, Ab55, Ab136, Ab39, Ab159, Ab151, Ab139, Ab107, Ab36, Ab193, Ab115, Ab106, Ab13f8, Ab127, Ab165, Ab155, Ab19, Ab6, Ab187, Ab179, Ab65, Ab114, Ab102, Ab94, Ab163, Ab110, Ab80, Ab92, Ab117, Ab162, Ab121, Ab195, Ab84, Ab161, Ab198, Ab24, Ab98, Ab116, Ab174, Ab196, Ab51, Ab91, Ab185, Ab23, Ab7, Ab95, Ab100, Ab140, Ab145, Ab150, Ab168, Ab54, Ab77, Ab43, Ab160, Ab82, Ab189, Ab17, Ab103, Ab18, Ab130, Ab132, Ab134, Ab144; ARG Clones: Ab2, Ab47, Ab49, Ab31, Ab53, Ab40, Ab5, Ab9, Ab48, Ab4, Ab10, Ab37, Ab33, Ab42, Ab45; ARV Clones: Ab44, Ab97, Ab81, Ab188, Ab186, Ab62, Ab57, Ab192, Ab73, Ab60, Ab28, Ab32, Ab78, Ab14, Ab152, Ab72, Ab137, Ab128, Ab169, Ab87, Ab74, Ab172, Ab153, Ab120, Ab13, Ab113, Ab16, Ab56, Ab129, Ab50, Ab90, Ab99, Ab3, Ab148, Ab124, Ab22, Ab41, Ab119, Ab157, Ab27, Ab15, Ab191, Ab190, Ab79, Ab181, Ab146, Ab167, Ab88, Ab199, Ab71, Ab85, Ab59, Ab141, Ab68, Ab143, Ab46, Ab197, Ab175, Ab156, Ab63, Ab11, Ab182, Ab89, Ab8, Ab101, Ab25, Ab154, Ab21, Ab111, Ab118, Ab173, Ab38, Ab76, Ab131, Ab1, Ab67, Ab70, Ab170, Ab30, Ab93, Ab142, Ab104, Ab112, Ab35, Ab126, and Ab125 (Rigel Pharmaceuticals, Inc.); CASC-674 (Seattle Genetics); antibody clones 2, 20, 3, 5, 13, 13A, 13B, 13C, 13D, 14, 16, 16C, 16D, 16E, 18, 21, 22, 25, 25A, 25B, 25C, 25D, 25E, 27, 54, 13 IgG2a afucosylated, 13 hIgG1 wild-type, and 13 LALA-PG (Seattle Genetics); JS006 (Shanghai Junshi Biosciences Ltd.); anti-TIGIT Fc antibody and bispecific antibody PD1×TIGIT (Xencor), antibody clone VSIG9 #1 (Vsig9.01) and 258-CS1 #4 (#4) (Yissum Research Development Company of The Hebrew University Of Jerusalem Ltd.); YH29143 (Yuhan Co, Ltd.); antibody clones S02, S03, S04, S05, S06, S11, S12, S14, S19, S32, S39, S43, S62, S64, F01, F02, F03, F04, 32D7, 101H3, 10A7, and 1F4 (Yuhan Co, Ltd.); anti-zB7R1 clones 318.4.1.1 (E9310), 318.28.2.1 (E9296), 318.39.1.1 (E9311), 318.59.3.1 (E9400), and 318.77.1.10 (ZymoGenetics, Inc).

In some embodiments, the anti-TIGIT antibody is selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT). ASP874 (PTZ-201) is an anti-TIGIT monoclonal antibody described in PCT Pub. No. WO2018183889A1 and US Pub. No. 2020/0095324. BGB-A1217 is an anti-TIGIT antibody as described in PCT Pub. No. WO2019129261A1. BMS-986207 (ONO-4686) is an anti-TIGIT antibody as described in PCT Pub. No. WO2016106302A9, U.S. Pat. No. 10,189,902 and US Pub. No. 2019/0112375. COM902 (CGEN-15137) is an anti-TIGIT antibody as described in PCT Pub. No. WO2018033798A1 and U.S. Pat. Nos. 10,213,505 and 10,124,061. IBI939 is an anti-TIGIT antibody as described in PCT Pub. No. WO2020020281A1. EOS884448 (EOS-448) is an anti-TIGIT antibody described in PCT Pub. No. WO2019023504A1. Domvanalimab (AB154) is an anti-TIGIT monoclonal antibody as described in PCT Pub. No. WO2017152088A1 and U.S. Pat. No. 10,537,633. Vibostolimab (MK-7684) is an anti-TIGIT antibody described in PCT Pub. Nos. WO2016028656A1, WO2017030823A2, WO2018204405A1, and/or WO2019152574A1, U.S. Pat. No. 10,618,958, and US Pub. No. 2018/0371083. SEA-TGT (SGN-TGT) is an anti-TIGIT antibody as described in PCT Pub. No. WO2020041541A2 and US Pub. No. 2020/0062859.

In some embodiments, the anti-TIGIT antagonist antibody is tiragolumab (CAS Registry Number: 1918185-84-8). Tiragolumab (Genentech) is also known as MTIG7192A, RG6058 or RO7092284. Tiragolumab is an anti-TIGIT antagonistic monoclonal antibody described in PCT Pub. No. WO2003072305A8, WO2004024068A3, WO2004024072A3, WO2009126688A2, WO2015009856A2, WO2016011264A1, WO2016109546A2, WO2017053748A2, and WO2019165434A1, and US Pub. Nos. 2017/0044256, 2017/0037127, 2017/0145093, 2017/260594, 2017/0088613, 2018/0186875, 2019/0119376 and U.S. Pat. Nos. 9,873,740B2, 10,626,174B2, 10,611,836B2, 9,499,596B2, 8,431,350B2, 10,047,158B2, and 10,017,572B2.

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, IBI939, EOS884448 (EOS-448), domvanalimab (AB154), vibostolimab (MK-7684), and SEA-TGT (SGN-TGT).

### VI. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used for treating a subject having a cancer (e.g., GC or GEJC (e.g., inoperable, locally advanced, metastatic, or advanced GC or GEJC) or rectal cancer (e.g., LARC)).

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:

(a) a heavy chain variable region (VH) comprising the amino acid sequence:
(SEQ ID NO: 9)
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSS,
and
(b) the light chain variable region (VL) comprising the amino acid sequence:
(SEQ ID NO: 10)
DIQMTQSPSSLSASVGDRVTITCRASQDVSTAVAWYQQKPGKAPKLLIYSASFLYSGVPSRFSGSGSGTD
FTLTISSLQPEDFATYYCQQYLYHPATFGQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKFN
WYVDGVEVHNAKTKPREEQYASTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPG,
and
(b) the light chain amino acid sequence:
(SEQ ID NO: 2)
DIQMTQSPSSLSASVGDRVTITCRASQDVSTAVAWYQQKPGKAPKLLIYSASFLYSGVPSRFSGSGSGTD
FTLTISSLQPEDFATYYCQQYLYHPATFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### VII. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. Further provided herein are pharmaceutical compositions and formulations comprising an anti-TIGIT antagonist antibody (e.g., tiragolumab) and, optionally, a pharmaceutically acceptable carrier. The disclosure also provides pharmaceutical compositions and formulations comprising a PD-1 axis binding antagonist (e.g., atezolizumab) and an anti-TIGIT antagonist antibody, and optionally, a pharmaceutically acceptable carrier.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary tiragolumab formulation comprises a histidine solution containing polysorbate 20, sucrose, L-methionine, and WFI. Tiragolumab may be provided in a 15-mL vial containing 10 ml of tiragolumab drug product at an approximate concentration of tiragolumab antibody of 60 mg/mL.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VIII. Articles of Manufacture or Kits

In another aspect, provided herein is an article of manufacture or a kit comprising a PD-1 axis binding antagonist (e.g., atezolizumab) and/or an anti-TIGIT antagonist antibody (e.g., tiragolumab). In some instances, the article of manufacture or kit further comprises a package insert comprising instructions for using the anti-TIGIT antagonist antibody in combination with the PD-1 axis binding antagonist to treat or delay progression of GC or GEJC (e.g., inoperable, locally advanced, metastatic, or advanced GC or GEJC) or rectal cancer (e.g., LARC) in a subject. In some instances, the kit further comprises capecitabine and oxaliplatin; for example, in some aspects, provided herein is an article of manufacture or a kit comprising a PD-1 axis binding antagonist (e.g., atezolizumab), an anti-TIGIT antagonist antibody (e.g., tiragolumab), capecitabine, and oxaliplatin, e.g., wherein the article of manufacture or kit further comprises a package insert comprising instructions for using the anti-TIGIT antagonist antibody in combination with the PD-1 axis binding antagonist, capecitabine, and oxaliplatin to treat or delay progression of GC or GEJC (e.g., inoperable, locally advanced, metastatic, or advanced GC or GEJC) in a subject. Any of the PD-1 axis binding antagonists and/or anti-TIGIT antagonist antibodies described herein may be included in the article of manufacture or kits.

In another embodiment of the invention, a kit is provided comprising a PD-1 axis binding antagonist for use in combination with an anti-TIGIT antagonist antibody for treating a subject having a cancer according to any of the methods described herein. In some instances, the kit further comprises the anti-TIGIT antagonist antibody. In some instances, the article of manufacture or kit further comprises a package insert comprising instructions for using the PD-1 axis binding antagonist in combination with anti-TIGIT antagonist antibody (e.g., tiragolumab) to treat or delay progression of a cancer in a subject. In some instances, the kit further comprises capecitabine and oxaliplatin.

In another embodiment, a kit comprises tiragolumab for use in combination with atezolizumab for treating a subject having a cancer according to any of the methods described herein. In some embodiments, the kit further comprises atezolizumab. In some instances, the article of manufacture or kit further comprises package insert comprising instructions for using tiragolumab in combination with atezolizumab to treat or delay progression of a cancer in a subject. In some instances, the kit further comprises capecitabine and oxaliplatin.

In another embodiment, a kit comprises atezolizumab for use in combination with tiragolumab for treating a subject having a cancer according to any of the methods described herein. In some embodiments, the kit further comprises tiragolumab. In some instances, the article of manufacture or kit further comprises package insert comprising instructions for using atezolizumab in combination with tiragolumab to treat or delay progression of cancer in a subject. In some instances, the kit further comprises capecitabine and oxaliplatin.

In some instances, the PD-1 axis binding antagonist and the anti-TIGIT antagonist antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the PD-1 axis binding antagonists and/or anti-TIGIT antagonist antibodies described herein may be included in the article of manufacture or kits. Any of the articles of manufacture or kits may include instructions to administer a PD-1 axis binding antagonist and/or an anti-TIGIT antagonist antibody to a subject in accordance with any of the methods described herein, e.g., any of the methods set forth in Section II above.

### EXAMPLES

### Example 1: A Phase Ib/II, Open-Label, Multicenter, Randomized Umbrella Study Evaluating the Efficacy and Safety of Multiple Treatment Combinations in Patients with Gastric or Gastroesophageal Junction Carcinoma

Gastric carcinoma (GC) is the fifth leading cancer and the fourth leading cause of cancer-related deaths globally. In China, GC remains the second in terms of incidence among all malignancies, just below lung cancer (Chen et al. Chin J Cancer Res. 30: 1-12, 2018). With respect to mortality, GC is ranked third, preceded by lung cancer and liver cancer, whereas the mortality among males and females is ranked third and second, respectively. New treatment options are needed to improve survival and response as well as decrease toxicity in the first-line treatment setting of GC and gastroesophageal junction carcinoma (GEJC).

This example describes a Phase Ib/II, open-label, multicenter, randomized umbrella study (YO43408) in patients with advanced GC or GEJC. The study is designed to accelerate the development of treatment combinations by identifying early signals and establishing proof-of-concept clinical data in patients with GC or GEJC. The study is designed with the flexibility to open new treatment arms as new treatments become available, close existing treatment arms that demonstrate minimal clinical activity or unacceptable toxicity, and modify the patient population (e.g., with regard to prior anti-cancer treatment or biomarker status).

### A. Overview of Study Design

The study evaluates the efficacy, safety, and pharmacokinetics of multiple treatment combinations in patients with advanced GC or GEJC. Specific objectives and corresponding endpoints for the study are outlined below for Stage 1 (see Table 1) and Stage 2 (see Table 2).

TABLE 1
Objectives and Corresponding Endpoints for Stage 1

| | Corresponding Endpoints |
|---|---|
| Primary Efficacy Objective | |
| To evaluate the efficacy of | ORR, defined as the proportion of patients with a complete response |
| treatment combinations | or a partial response on two consecutive occasions ≥4 weeks apart |
| during Stage 1 | during Stage 1, as determined by the investigator according to |
| | RECIST v1.1. |
| Secondary Efficacy | |
| Objective | |
| To evaluate the efficacy of | PFS after randomization, defined as the time from randomization to |
| treatment combinations | the first occurrence of disease progression or death from any cause |
| during Stage 1 | (whichever occurs first) in Stage 1, as determined by the investigator |
| | according to RECIST v1.1. |
| | OS after randomization, defined as the time from randomization to |
| | death from any cause. |
| | OS at specific timepoints (e.g., 6 and 12 months). |
| | DOR, defined as the time from the first occurrence of a documented |
| | objective response to disease progression or death from any cause |
| | (whichever occurs first) in Stage 1, as determined by the investigator |
| | according to RECIST v1.1. |
| | Disease control, defined as stable disease for ≥12 weeks or a |
| | complete or a partial response, as determined by the investigator |
| | according to RECIST v1.1. |
| | Objective response in patients with PD-L1-positive and/or TIGIT- |
| | positive tumors, as assessed by IHC. |
| Safety Objective | |
| To evaluate the safety of | Incidence and severity of adverse events, with severity of all events |
| treatment combinations | graded according to NCI CTCAE v5.0 and severity for CRS also |
| during Stage 1 | graded according to the ASTCT CRS Consensus Grading Scale. |
| Exploratory | |
| Pharmacokinetic | |
| Objectives | |
| To characterize the PK | Plasma or serum concentrations of each drug (as appropriate) at |
| profile of drugs that are | specified timepoints. |
| administered as part of a | |
| treatment combination | |
| during Stage 1 | |
| To evaluate potential | Relationship between plasma or serum concentrations or PK |
| relationships between drug | parameters for each drug (as appropriate on the basis of available |
| exposure during Stage 1 and | data) and efficacy endpoints. |
| the efficacy and safety of | Relationship between plasma or serum concentrations or PK |
| treatment combinations | parameters for each drug (as appropriate on the basis of available |
| | data) and safety endpoints. |
| Exploratory | |
| Immunogenicity | |
| Objectives | |
| To evaluate the immune | For drugs for which ADA formation is measured: presence of ADAs |
| response to drugs that are | during the study relative to the presence of ADAs at baseline. |
| administered as part of a | |
| treatment combination | |
| during Stage 1 | |
| To evaluate potential effects | For drugs for which ADA formation is measured: relationship |
| of ADAs during Stage 1 | between ADA status and efficacy, safety, or PK endpoints |
| Biomarker Objective | |
| To identify biomarkers during | Relationship between biomarkers in blood and tumor tissue and |
| Stage 1 that are predictive of | efficacy, safety, PK, immunogenicity, or other biomarker endpoints |
| response to study treatment | |
| (i.e., predictive biomarkers), | |
| are associated with | |
| progression to a more | |
| severe disease state (i.e., | |
| prognostic biomarkers), are | |
| associated with resistance to | |
| study treatment, are | |
| associated with susceptibility | |
| to developing adverse | |
| events (i.e., safety | |
| biomarkers), can provide | |
| evidence of study treatment | |
| activity (i.e., | |
| pharmacodynamic | |
| biomarkers), or can increase | |
| the knowledge and | |
| understanding of disease | |
| biology | |

ADA = anti-drug antibody; ASTCT = American Society for Transplantation and Cellular Therapy; CRS = cytokine-release syndrome; DOR = duration of response; DOR = duration of response; NCI CTCAE v5.0 = National Cancer Institute Common Terminology Criteria for Adverse Events, Version 5.0; ORR = objective response rate; OS = overall survival; PD-L1 = programmed death-ligand 1; PFS = progression-free survival; PK = pharmacokinetic; RECIST v1.1 = Response Evaluation Criteria in Solid Tumors, Version 1.1; TIGIT = T-cell immunoreceptor with Ig and ITIM domains.
Note:
Overall response at a single timepoint is assessed by the investigator using RECIST v1.1.

TABLE 2
Objectives and Corresponding Endpoints for Stage 2

| | Corresponding Endpoints |
|---|---|
| Exploratory Efficacy | |
| Objective | |
| To evaluate the efficacy of | Pathological ORR, defined as a complete response or a partial |
| treatment combinations | response on two consecutive occasions ≥4 weeks apart during |
| during Stage 2 | Stage 2, as determined by the investigator according to RECIST |
| | v1.1. |
| | PFS after initiation of Stage 2, defined as the time from initiation of |
| | Stage 2 to the first occurrence of disease progression or death from |
| | any cause (whichever occurs first), as determined by the investigator |
| | according to RECIST v1.1. |
| | OS after initiation of Stage 2, defined as the time from initiation of |
| | Stage 2 to death from any cause |
| | DOR, defined as the time from the first occurrence of a documented |
| | objective response during Stage 2 to disease progression or death |
| | from any cause (whichever occurs first), as determined by the |
| | investigator according to RECIST v1.1. |
| | Disease control, defined as stable disease for ≥12 weeks or a |
| | complete or a partial response, as determined by the investigator |
| | according to RECIST v1.1. |
| Safety Objective | |
| To evaluate the safety of | Incidence and severity of adverse events, with severity of all events |
| treatment combinations | graded according to NCI CTCAE v5.0 and severity for CRS also |
| during Stage 2 | graded according to the ASTCT CRS Consensus Grading Scale. |
| Exploratory | |
| Pharmacokinetic | |
| Objectives | |
| To characterize the PK | Plasma or serum concentrations of each (as appropriate) drug at |
| profile of drugs that are | specified timepoints. |
| administered as part of a | |
| treatment combination during | |
| Stage 2 | |
| To evaluate potential | Relationship between plasma or serum concentrations or PK |
| relationships between drug | parameters for each drug (as appropriate on the basis of available |
| exposure during Stage 2 and | data) and efficacy endpoints. |
| the efficacy and safety of | Relationship between plasma or serum concentrations or PK |
| immunotherapy-based | parameters for each drug (as appropriate on the basis of available |
| treatment combinations | data) and safety endpoints. |
| Exploratory | |
| Immunogenicity | |
| Objectives | |
| To evaluate the immune | For drugs for which ADA formation is measured: presence of ADAs |
| response to drugs that are | during the study relative to the presence of ADAs at baseline. |
| administered as part of a | |
| treatment combination during | |
| Stage 2 | |
| To evaluate potential effects | For drugs for which ADA formation is measured: relationship |
| of ADAs during Stage 2 | between ADA status and efficacy, safety, or PK endpoints. |
| Biomarker Objective | |
| To identify biomarkers during | Relationship between biomarkers in blood and tumor tissue and |
| Stage 2 that are predictive of | efficacy, safety, PK, immunogenicity, or other biomarker endpoints. |
| response to study treatment | |
| (i.e., predictive biomarkers), | |
| are associated with | |
| progression to a more severe | |
| disease state (i.e., prognostic | |
| biomarkers), are associated | |
| with resistance to study | |
| treatment, are associated | |
| with susceptibility to | |
| developing adverse events | |
| (i.e., safety biomarkers), can | |
| provide evidence of study | |
| treatment activity (i.e., | |
| pharmacodynamic | |
| biomarkers), or can increase | |
| the knowledge and | |
| understanding of disease | |
| biology | |

ADA = anti-drug antibody; ASTCT = American Society for Transplantation and Cellular Therapy; CRS = cytokine-release syndrome; DOR = duration of response; DOR = duration of response; NCI CTCAE v5.0 = National Cancer Institute Common Terminology Criteria for Adverse Events, Version 5.0; ORR = objective response rate; OS = overall survival; PD-L1 = programmed death-ligand 1; PFS = progression-free survival; PK = pharmacokinetic; RECIST v1.1 = Response Evaluation Criteria in Solid Tumors, Version 1.1.
Note:
Overall response at a single timepoint is assessed by the investigator using RECIST v1.1.

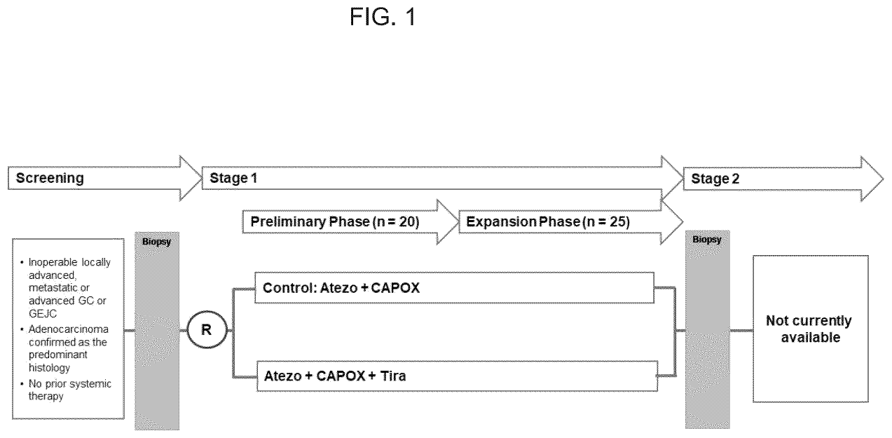

Cohort 1 enrolls patients with inoperable, locally advanced, metastatic, or advanced GC or GEJC, with adenocarcinoma confirmed as the predominant histology, who have not received prior systemic therapy for advanced or metastatic disease (see FIG. 1). Eligible patients are initially randomly assigned to one of the two treatment arms (Stage 1). Patients who experience loss of clinical benefit or unacceptable toxicity during Stage 1 may be eligible to receive treatment with a different treatment combination (Stage 2).

### Stage 1

During Stage 1, patients are randomly assigned to a control arm (atezolizumab in combination with capecitabine and oxaliplatin [CAPOX] [Atezo+CAPOX]) or an experimental arm consisting of atezolizumab and CAPOX in combination with tiragolumab (Atezo+CAPOX+Tira). Details on the treatment regimens for Stage 1 are provided in Table 3.

Approximately 40-90 patients are enrolled during Stage 1. Enrollment in the experimental arm takes place in two phases: a preliminary phase, followed by an expansion phase. Approximately 20 patients are enrolled during the preliminary phase. If clinical activity is observed in the experimental arm during the preliminary phase, approximately 25 additional patients may be enrolled in that arm during the expansion phase. The Sponsor may decide to delay or suspend enrollment within a given treatment arm. If the experimental arm shows minimal clinical activity or unacceptable toxicity, then it does not undergo expansion. Additional patients may be enrolled to ensure balance across treatment arms with respect to demographic and baseline characteristics, including potential predictive biomarkers, to enable further subgroup analyses. New experimental arms may be added during the study.

Patients in Stage 1 are randomly assigned to experimental arms or the control arm, and the randomization ratio depends on the number of experimental arms that are open for enrollment (e.g., if an arm is added or enrollment in an arm is suspended pending analysis of results from the preliminary phase), with the stipulation that the likelihood of being allocated to the control arm is no more than 50%. The treatment regimen in the control arm may change with emerging data to reflect the evolving standard-of-care treatments. Randomization takes into account arm-specific exclusion criteria. Patients are ineligible for a specific arm if they meet any of the exclusion criteria outlined for that arm.

TABLE 3
Stage 1 Treatment Regimens

| | | Number of Patients | |
|---|---|---|---|
| | | Preliminary | Expansion |
| Arm Name | Study Treatment ^{a} | Phase | Phase ^{b} |
| Control (Atezo + | Atezolizumab plus | Variable ^{d} | |
| CAPOX) ^{c} | capecitabine plus | | |
| | oxaliplatin | | |
| Atezo + | Atezolizumab plus | 20 | 25 |
| CAPOX + Tira | capecitabine plus | | |
| | oxaliplatin plus | | |
| | tiragolumab | | |

Atezo = atezolizumab; CAPOX = capecitabine + oxaliplatin; Tira = tiragolumab
^{a} The Sponsor may decide to delay or suspend enrollment within a given treatment arm. Thus, all listed experimental arms may not be open for enrollment at the same time.
^{b} If clinical activity is observed in an experimental arm during the preliminary phase, approximately 25 additional patients may be enrolled in that arm during the expansion phase. Experimental arms with minimal clinical activity or unacceptable toxicity do not undergo expansion.
^{c} The treatment regimen in the control arm may change with emerging data to reflect the evolving standard-of-care treatments.
^{d} The randomization ratio depends on the number of experimental arms that are open for enrollment (e.g., if an arm is added or enrollment in an arm is suspended pending analysis of results from the preliminary phase), with the stipulation that the likelihood of being allocated to the control arm is no more than 50%.

Patients in the control and experimental arms continue to receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease).

Because of the possibility of an initial increase in tumor burden caused by immune cell infiltration in the setting of a T-cell response (termed pseudoprogression) with atezolizumab and other cancer immunotherapies (CITs), radiographic progression per Response Evaluation Criteria in Solid Tumors, Version 1.1 (RECIST v1.1) may not be indicative of true disease progression. In the absence of unacceptable toxicity, patients who meet criteria for disease progression per RECIST v1.1 while receiving treatment with a CIT-based combination are permitted to continue treatment if they meet all of the following criteria:
Evidence of clinical benefit, as determined by the investigator following a review of all available data.
Absence of symptoms and signs (including laboratory values, such as new or worsening hypercalcemia) indicating unequivocal progression of disease.
Absence of decline in Eastern Cooperative Oncology Group (ECOG) Performance Status that can be attributed to disease progression.
Absence of tumor progression at critical anatomical sites (e.g., leptomeningeal disease) that cannot be managed by protocol-allowed medical interventions.

### Stage 2

Patients in a control or experimental arm who experience loss of clinical benefit as determined by the investigator (as described above) during Stage 1 are given the option of receiving a different treatment combination during Stage 2, as outlined in Table 4, provided they meet eligibility criteria (as listed above) and a Stage 2 arm is open for enrollment. Patients who experience unacceptable toxicity during Stage 1 may be eligible to receive treatment during Stage 2.

TABLE 4
Stage 2 Treatment Regimens
Study Treatment

No Stage 2 treatment currently available

Stage 2 treatment must begin within 3 months after a patient has experienced loss of clinical benefit or unacceptable toxicity in Stage 1 and continues until unacceptable toxicity or loss of clinical benefit as determined by the investigator. However, it is recommended that patients begin Stage 2 treatment as soon as possible. The Sponsor may also decide to discontinue enrollment in the Stage 2 treatment arms on the basis of a review of all available safety data, preliminary efficacy data, and supportive information (e.g., biomarker research data), as appropriate.

### B. End of Study and Length of Study

The end of this study is defined as the date when the last patient completes the last visit, including survival follow-up visits conducted by telephone or in the clinic. In addition, the Sponsor may decide to terminate the study at any time.

The total length of the study, from screening of the first patient to the end of the study, is expected to be approximately 3-5 years.

### C. Rationale for Study Design

### Rationale for Patient Population

This study enrolls patients with inoperable locally advanced, metastatic, or advanced GC or

GEJC, with adenocarcinoma confirmed as the predominant histology, who have not received prior systemic therapy for their advanced or metastatic disease.

The current standard-of-care treatment for the first-line treatment of patients with HER2-negative GC and GEJC with cytotoxic chemotherapy is typically palliative in intent and results in poor prognosis, with median OS duration of 8 to 14 months across Asia, the United States, and Europe (Cunningham et al. N Engl J Med. 358: 36-46, 2008; Ohtsu et al. J Clin Oncol. 30: 3968-3976, 2011; Yamada et al. Ann Oncol. 26: 141-148, 2015). In patients with GC and GEJC with a PD-L1 CPS≥5, nivolumab in combination with chemotherapy resulted in additional survival benefit compared with chemotherapy alone. However, the survival benefit in the all-randomized population was marginal and the treatment effect in the PD-L1 CPS<5 population remained unclear (Moehler et al. Ann Oncol. 31: S1191, 2020). Moreover, cytotoxic agents given as treatment backbone are accompanied by high toxicity, with Grade 3 and 4 toxicities reported in up to 77% of patients treated with platinum-doublet regimens and >80% of patients with triplet regimens (Van Cutsem et al. J Clin Oncol. 24: 4991-4997, 2006; Cunningham et al. N Engl J Med. 358: 36-46, 2008; Ohtsu et al. J Clin Oncol. 30: 3968-3976, 2011; Lordick et al. Lancet Oncol. 14: 490-499, 2013). Therefore, despite the recently demonstrated clinical benefit of nivolumab plus chemotherapy in patients with a PD-L1 CPS≥5, there remains a high unmet medical need for treatment-naive patients with inoperable, locally advanced, metastatic, or advanced GC and GEJC, requiring further evaluation of novel, more efficacious treatment combinations to improve survival and response as well as decreased toxicity in the first-line setting.

### Rationale for Immunotherapy-Based Treatment Beyond Initial Radiographic Progression

In studies of immunotherapeutic agents, complete response, partial response, and stable disease have each been shown to occur after radiographic evidence of an apparent increase in tumor burden. This initial increase in tumor burden caused by immune cell infiltration in the setting of a T-cell response has been termed pseudoprogression (Hales et al. Ann Oncol. 21: 1944-1951, 2010). In Study PCD4989g, evidence of tumor growth followed by a response was observed in several tumor types. In addition, in some responding patients with radiographic evidence of progression, biopsies of new lesions or areas of new growth in existing lesions revealed immune cells and no viable cancer cells. Because of the potential for a response after pseudoprogression, this study allows patients randomly allocated to immunotherapy-based treatment arms to continue combination treatment after apparent radiographic progression per RECIST v1.1, provided the benefit-risk ratio is judged by the investigator to be favorable. Patients should be discontinued for unacceptable toxicity or loss of clinical benefit as determined by the investigator after an integrated assessment of radiographic and biochemical data, local biopsy results (if available), and clinical status.

### D. Inclusion Criteria

### Inclusion Criteria for Stage 1

Patients must meet all of the following criteria to qualify for Stage 1:
Age ≥18 years at the time of signing Informed Consent Form.
ECOG Performance Status of 0 or 1.
Inoperable, locally advanced, metastatic, or advanced GC or GEJC, with adenocarcinoma confirmed as the predominant histology.
No prior systemic treatment (including systemic investigational agents or HER2 inhibitors) for advanced or metastatic disease.
   Prior adjuvant or neoadjuvant chemotherapy, radiotherapy, or chemoradiotherapy for GC and GEJC are permitted as long as the last administration of the last dose (whichever was given last) occurred at least 6 months prior to randomization. Palliative radiotherapy is allowed and must be completed 2 weeks prior to randomization.
   Prior treatment with herbal therapies, including traditional Chinese medicines, with anti-cancer activity noted in the label are allowed, provided that these medications are discontinued prior to randomization.
Life expectancy ≥3 months, as determined by the investigator.
Availability of a representative tumor specimen that is suitable for determination of programmed death-ligand 1 (PD-L1) and/or additional biomarker status by central testing.
   Baseline tumor tissue samples are collected from all patients, preferably by means of a biopsy performed at study entry. If a biopsy is not deemed feasible by the investigator, archival tumor tissue may be submitted after Medical Monitor approval has been obtained, provided that the tissue was obtained from a previous surgery or biopsy within 6 months prior to enrollment and that the patient has not received any anti-cancer therapy since the time of the procedure. Patients with archival tumor specimens ≥6 months old available at baseline may be eligible upon discussion with the Medical Monitor if a recent biopsy is not clinically feasible.
   A formalin-fixed, paraffin-embedded tumor specimen at least 18 slides containing unstained, freshly cut serial sections must be submitted along with an associated pathology report. Patients with <18 slides available may still be eligible for the study.
Patients whose tumors are without HER2 amplification documented by fluorescence in situ hybridization or in situ hybridization or are negative by immunohistochemistry (IHC) 0 or +1 on previously collected and assessed tumor tissue at initial diagnosis of disease by local laboratory testing.

### Inclusion Criteria for Stage 1 and Stage 2

Patients must meet all of the following criteria to qualify for Stage 1 and to qualify for Stage 2:
Ability to comply with the study protocol, in the investigator's judgment.
Measurable disease (at least one target lesion) according to RECIST v1.1.
Adequate hematologic and end-organ function, defined by the following laboratory test results, obtained within 14 days prior to initiation of study treatment:
   Absolute neutrophil count (ANC)≥1.5×10⁹/L (1500/μL) without granulocyte colony-stimulating factor support.
   Lymphocyte count ≥0.5×10⁹/L (500/μL).
   Platelet count ≥100×10⁹/L (100,000/μL) without transfusion.
   Hemoglobin ≥90 g/L (9.0 g/dL) without transfusion.
   Aspartate aminotransferase (AST), alanine aminotransferase (ALT), and alkaline phosphatase (ALP)≤2.5× upper limit of normal (ULN) with the following exceptions:
      Patients with documented liver metastases: AST and/or ALT≤5×ULN.
      Patients with documented liver or bone metastases: ALP≤5×ULN.
   Bilirubin ≤1.5×ULN.
   Creatinine clearance ≥30 mL/min (calculated using the Cockcroft-Gault formula).
   Serum albumin ≥25 g/L (2.5 g/dL).
   For patients not receiving anticoagulation: international normalized ratio (INR) and activated partial thromboplastin time (aPTT)≤1.5×ULN.
   For patients receiving therapeutic anticoagulation: stable anticoagulant regimen.
Patients without hepatitis B virus (HBV) infection at screening.
   For patients with a positive hepatitis B surface antigen (HBsAg) test and/or a positive total hepatitis B core antibody test in the absence of a positive hepatitis B surface antibody test at screening: HBV DNA<500 IU/mL.
   Patients with detectable HBV DNA should be managed per institutional guidelines. Initiation of anti-HBV therapy should be ≥14 days prior to initiation of study treatment, and patients should be willing to continue anti-HBV therapy for the duration of study treatment and longer per institutional guidelines.
Negative hepatitis C virus (HCV) antibody test at screening, or positive HCV antibody test followed by a negative HCV RNA test at screening.
   The HCV RNA test is performed only for patients who have a positive HCV antibody test.
Negative human immunodeficiency virus (HIV) test at screening.
For women of childbearing potential: agreement to remain abstinent (refrain from heterosexual intercourse) or use contraception, and agreement to refrain from donating eggs.
For men: agreement to remain abstinent (refrain from heterosexual intercourse) or use contraception, and agreement to refrain from donating sperm.

### Inclusion Criteria for Stage 2

Patients must meet all of the following criteria to qualify for Stage 2:
ECOG Performance Status of 0 or 1.
Ability to initiate Stage 2 treatment within 3 months after experiencing unacceptable toxicity not related to atezolizumab or loss of clinical benefit as determined by the investigator (while receiving Stage 1 treatment.
Availability of a tumor specimen from a biopsy performed upon discontinuation of Stage 1 (if deemed clinically feasible by the investigator) because of unacceptable toxicity, disease progression per RECIST v1.1, or loss of clinical benefit as determined by the investigator.

### E. Exclusion Criteria

Patients are excluded from enrollment in specific arms during Stage 1 or enrollment during Stage 2 if they meet any of the following criteria, as specified by treatment arm below:

### Exclusion Criteria for Stage 1

Patients who meet any of the following criteria are excluded from Stage 1:
Prior treatment with CD137 agonists or immune checkpoint blockade therapies, including but not limited to, anti-CTLA-4, anti-PD-1, anti-PD-L1, and anti-TIGIT therapeutic antibodies.
Treatment with investigational therapy within 28 days prior to initiation of study treatment.
Any contraindications to any of the study drugs of the chemotherapy regimen.
   Investigators should refer to local package insert of the chemotherapy drugs.
Eligible only for the control arm.

### Exclusion Criteria for Stage 1 and Stage 2

Patients who meet any of the following criteria are excluded from Stage 1 and from Stage 2:
Patients with a signet ring cells dominant carcinoma (>50% of the tumor).
Symptomatic, untreated, or actively progressing central nervous system (CNS) metastases. Asymptomatic patients with treated CNS lesions are eligible, provided that all of the following criteria are met:
   Measurable disease, per RECIST v1.1, must be present outside the CNS.
   The patient has no history of intracranial hemorrhage or spinal cord hemorrhage.
   The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
   The patient has no ongoing requirement for corticosteroids as therapy for CNS disease. Anti-convulsant therapy at a stable dose is permitted.
   Metastases are limited to the cerebellum or the supratentorial region (i.e., no metastases to the midbrain, pons, medulla, or spinal cord).
   There is no evidence of interim progression between completion of CNS-directed therapy and initiation of study treatment.
   Asymptomatic patients with CNS metastases newly detected at screening are eligible for the study after receiving radiotherapy or surgery, with no need to repeat the screening brain scan.
History of leptomeningeal disease.
Uncontrolled tumor-related pain.
   Patients requiring pain medication must be on a stable regimen at study entry.
   Symptomatic lesions (e.g., bone metastases or metastases causing nerve impingement) amenable to palliative radiotherapy should be treated prior to enrollment. Patients should be recovered from the side effects of radiation. There is no required minimum recovery period.
   Asymptomatic metastatic lesions that would likely cause functional deficits or intractable pain with further growth (e.g., epidural metastasis that is not currently associated with spinal cord compression) should be considered for locoregional therapy if appropriate prior to enrollment.
Uncontrolled pleural effusion, pericardial effusion, or ascites requiring recurrent drainage procedures (once monthly or more frequently).
   Patients with indwelling catheters (e.g., PLEURX®) are allowed.
Uncontrolled or symptomatic hypercalcemia (ionized calcium >1.5 mmol/L, calcium >12 mg/dl, or corrected calcium greater than ULN).
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barre syndrome, or multiple sclerosis, with the following exceptions:
   Patients with a history of autoimmune-related hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met:
      Rash must cover <10% of body surface area.
      Disease is well controlled at baseline and requires only low-potency topical corticosteroids.
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on the screening chest computed tomography (CT) scan.
   History of radiation pneumonitis in the radiation field (fibrosis) is permitted.
Active tuberculosis.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Major surgical procedure, other than for diagnosis, within 4 weeks prior to initiation of study treatment, or anticipation of need for a major surgical procedure during the study.
History of malignancy other than GC or GEJC within 2 years prior to initiation of study treatment, with the exception of malignancies with a negligible risk of metastasis or death (e.g., 5-year overall survival rate >90%), such as adequately treated carcinoma in situ of the cervix, non-melanoma skin carcinoma, localized prostate cancer, ductal carcinoma in situ, or Stage I uterine cancer.
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to initiation of study treatment.
   Patients receiving prophylactic antibiotics (e.g., to prevent a urinary tract infection or chronic obstructive pulmonary disease exacerbation) are eligible for the study.
Prior allogeneic stem cell or solid organ transplantation.
Any other disease, metabolic dysfunction, physical examination finding, or clinical laboratory finding that contraindicates the use of an investigational drug, may affect the interpretation of the results, or may render the patient at high risk from treatment complications.
Pregnancy or breastfeeding, or intending to become pregnant during the study.
   Women of childbearing potential must have a negative serum pregnancy test result within 14 days prior to initiation of study treatment.
Treatment with a live, attenuated vaccine within 4 weeks prior to initiation of study treatment, or anticipation of need for such a vaccine during atezolizumab or tiragolumab treatment, within 5 months after the final dose of atezolizumab, or 90 days after the final dose of tiragolumab, whichever is later.
History of severe allergic anaphylactic reactions to chimeric or humanized antibodies or fusion proteins.
Known hypersensitivity to Chinese hamster ovary cell products or recombinant human antibodies.
Known allergy or hypersensitivity to any of the study drugs or any of their excipients.
Treatment with systemic immunostimulatory agents (including, but not limited to, interferon and interleukin-2) within 4 weeks or 5 drug-elimination half-lives (whichever is longer) prior to initiation of study treatment.
Treatment with systemic immunosuppressive medication (including, but not limited to, corticosteroids, cyclophosphamide, azathioprine, methotrexate, thalidomide, and anti-tumor necrosis factor-α agents) within 2 weeks prior to initiation of study treatment or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study after Medical Monitor confirmation has been obtained.
   Patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
For patients entering Stage 2: immunotherapy-related adverse events that have not resolved to Grade 1 or better or to baseline at the time of consent with the following exception:
   Patients with ongoing endocrine events that are adequately managed with supplemental therapy are eligible.

### Exclusion Criteria for Tiragolumab-Containing Arm

Patients who meet any of the following criteria are excluded from the tiragolumab-containing arm during Stage 1:
Prior treatment with an anti-TIGIT agent.
Active Epstein-Barr virus (EBV) infection or known or suspected chronic active EBV infection at screening.
   Patients with a positive EBV viral capsid antigen IgM test at screening are excluded from this arm. An EBV polymerase chain reaction (PCR) test should be performed as clinically indicated to screen for active infection or suspected chronic active infection. Patients with a positive EBV PCR test are excluded from this arm.

### F. Study Treatment

The investigational medicinal products for the study are atezolizumab and tiragolumab. The study is a randomized, open-label study.

For Stage 1, the study employs a permuted-block randomization method with dynamically changing randomization ratios to account for fluctuation in the number of treatment arms that are open for enrollment during the study. The randomization ratio depends on the number of experimental arms that are open for enrollment (e.g., if an arm is added or enrollment in an arm is suspended pending analysis of results from the preliminary phase), with the stipulation that the likelihood of being allocated to the control arm is no more than 50%. The randomization ratios may be altered to increase enrollment in a particular experimental arm that has demonstrated promising clinical activity.

Randomization takes into account general exclusion criteria and arm-specific exclusion criteria. If a patient is eligible only for the control arm, the patient is not enrolled in the study.

Patients who do not receive at least one dose of each drug for their assigned treatment regimen are not included in the efficacy analyses. Additional patients may be enrolled in Stage 1 to reach the target number of treated patients planned for analysis.

### Control Arm (Atezolizumab+CAPOX)

Patients in the control arm (atezolizumab plus CAPOX (Atezo+Capecitabine+Oxaliplatin)) receive treatment as outlined in Table 5 until unacceptable toxicity or loss of clinical benefit as determined by the investigator after an integrated assessment of radiographic and biochemical data, local biopsy results (if available), and clinical status (e.g., symptomatic deterioration such as pain secondary to disease). It is recommended that treatment is initiated no later than 7 calendar days after randomization.

TABLE 5
Treatment Regimen for Atezo + CAPOX Arm

| Cycle | Dose, Route, and Regimen |
|---|---|
| Length | (drugs listed in order of administration) |
| 21 days | Atezolizumab 1200 mg by IV infusion on Day 1 |
| | Capecitabine 1000 mg/m² orally twice daily on Days 1-14 ^{a} |
| | Oxaliplatin 130 mg/m² by IV infusion on Day 1 ^{a} |

Atezo = atezolizumab; CAPOX = capecitabine plus oxaliplatin
^{a} Treatment for up to six cycles.

Treatment with CAPOX continues for up to six cycles and patients are offered continued treatment with atezolizumab as long as they are experiencing clinical benefit in the opinion of the investigator. CAPOX or atezolizumab treatment may be temporarily suspended in patients who experience toxicity considered to be related to study treatment. If one component of the combination (atezolizumab or CAPOX) is discontinued, the other component may be continued if the patient is likely to derive clinical benefit. If oxaliplatin treatment is discontinued during the first six cycles, patients are encouraged to continue chemotherapy with capecitabine and atezolizumab, as long as they are experiencing clinical benefit in the opinion of the investigator and Medical Monitor. If capecitabine treatment is discontinued during the first six cycles, patients are encouraged to continue chemotherapy with oxaliplatin and atezolizumab, provided they are experiencing clinical benefit in the opinion of the investigator and Medical Monitor.

### Atezolizumab+CAPOX+Tira

Patients in atezolizumab plus CAPOX plus tiragolumab (Atezo+Capecitabine+Oxaliplatin+Tira) arm receive treatment as outlined in Table 6 until unacceptable toxicity or loss of clinical benefit as determined by the investigator after an integrated assessment of radiographic and biochemical data, local biopsy results (if available), and clinical status (e.g., symptomatic deterioration such as pain secondary to disease). It is recommended that treatment be initiated no later than 7 calendar days after randomization.

TABLE 6
Treatment Regimen for Atezo + CAPOX + Tira Arm

| Cycle | Dose, Route, and Regimen |
|---|---|
| Length | (drugs listed in order of administration) |
| 21 days | Atezolizumab 1200 mg by IV infusion on Day 1 |
| | Tiragolumab 600 mg by IV infusion on Day 1 |
| | Capecitabine 1000 mg/m² orally twice daily on Days 1-14 ^{a} |
| | Oxaliplatin 130 mg/m² by IV infusion on Day 1 ^{a} |

Atezo = atezolizumab; CAPOX = capecitabine plus oxaliplatin; Tira = tiragolumab
^{a} Treatment for up to six cycles.

Treatment with CAPOX continues for up to six cycles and patients are offered continued treatment with atezolizumab as long as they are experiencing clinical benefit in the opinion of the investigator. CAPOX, atezolizumab, and/or tiragolumab treatment may be temporarily suspended in patients experiencing toxicity considered to be related to study treatment. If atezolizumab is withheld or discontinued, tiragolumab should also be withheld or discontinued, but CAPOX may be continued if the patient is likely to derive clinical benefit. If CAPOX or tiragolumab is discontinued, the other drugs can be continued if the patient is likely to derive clinical benefit. If oxaliplatin treatment is discontinued during the first six cycles, patients are encouraged to continue capecitabine, atezolizumab, and tiragolumab as long as they are experiencing clinical benefit in the opinion of the investigator and Medical Monitor. If capecitabine treatment is discontinued during the first six cycles, patients are encouraged to continue oxaliplatin, atezolizumab, and tiragolumab as long as they are experiencing clinical benefit in the opinion of the investigator and Medical Monitor.

### G. Concomitant Therapy

Concomitant therapy consists of any medication (e.g., prescription drugs, over-the-counter drugs, vaccines, herbal or homeopathic remedies, nutritional supplements) used by a patient in addition to protocol-mandated study treatment from 7 days prior to initiation of study treatment to the treatment discontinuation visit.

Premedication with antihistamines, antipyretic medications, and/or analgesics may be administered for the second and subsequent atezolizumab infusions only, at the discretion of the investigator.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., oxygen saturation, or respiratory distress should be managed with supportive therapies as clinically indicated (e.g., supplemental oxygen and ß2-adrenergic agonists).

### Permitted Therapy

Patients are permitted to use the following therapies during the study:
Oral contraceptives with a failure rate of <1% per year.
Hormone-replacement therapy.
Prophylactic or therapeutic anticoagulation therapy.
Inactivated vaccinations.
Megestrol acetate administered as an appetite stimulant.
Mineralocorticoids (e.g., fludrocortisone).
Inhaled corticosteroids administered for chronic obstructive pulmonary disease or asthma.
Low-dose corticosteroids administered for orthostatic hypotension or adrenocortical insufficiency.
Palliative radiotherapy (e.g., treatment of known bony metastases or symptomatic relief of pain) as outlined below:
   Palliative radiotherapy is permitted, provided it does not interfere with the assessment of tumor target lesions (e.g., the lesion to be irradiated must not be the only site of measurable disease). Treatment with atezolizumab and CAPOX may be continued during palliative radiotherapy.
Radiotherapy to the brain as outlined below:
   Patients whose extracranial tumor burden is stable or responding to study treatment and who are subsequently found to have three or fewer brain metastases may receive radiotherapy to the brain (either stereotactic radiosurgery or whole-brain radiation therapy) provided that all of the following criteria are met:
      The patient has no evidence of progression or hemorrhage after completion of central nervous system (CNS)-directed therapy.
      The patient has no ongoing requirement for corticosteroids as therapy for CNS disease.
         Patients who require corticosteroid therapy for more than 7 days after completion of radiotherapy must be discontinued from study treatment.
      Anti-convulsant therapy, if required, is administered at a stable dose.

### H. Assessments

All patients are closely monitored for adverse events throughout the study, and adverse events are graded according to the National Cancer Institute Common Terminology Criteria for Adverse Events, Version 5.0 (NCI CTCAE v5.0). Severity for CRS is also graded according to the American Society for Transplantation and Cellular Therapy (ASTCT) CRS Consensus Grading Scale.

### Tumor and Response Evaluations

Patients undergo tumor assessments every 6 weeks (±1 week) during the first 48 weeks (from Day 1 of Cycle 1) and then every 12 weeks (±2 weeks) thereafter, regardless of dose delays, except in the case of patients who continue treatment after radiographic disease progression; such patients undergo tumor assessments every 6 weeks (±1 week) until loss of clinical benefit as determined by the investigator. Thus, tumor assessments continue according to schedule in patients who discontinue treatment for reasons other than loss of clinical benefit, even if they start a new non-protocol-specified anti-cancer therapy. Tumor assessments may be repeated at any time if progressive disease is suspected.

Baseline tumor assessments for Stage 2 must be performed within 28 days prior to initiation of Stage 2 treatment (i.e., Day 1 of Cycle 1). Tumor assessments performed prior to or at the time of unacceptable toxicity or loss of clinical benefit during Stage 1 may serve as baseline assessments for Stage 2, provided the tumor assessments are performed within 28 days prior to initiation of Stage 2 treatment.

All measurable and/or evaluable lesions are assessed and documented at screening (in both Stage 1 and Stage 2). Brain metastases treated with radiotherapy or surgery are not considered measurable or evaluable but are documented at screening as a site of metastatic disease. Tumor assessments performed as standard of care prior to obtaining informed consent and within 28 days prior to initiation of study treatment do not have to be repeated at screening.

All measurable and/or evaluable lesions identified at baseline should be re-assessed at subsequent tumor evaluations according to the schedule described above. Brain metastases identified at baseline that have been treated with radiotherapy or surgery are not considered measurable or evaluable unless there is suspected disease progression in the brain (i.e., the patient becomes symptomatic). Thus, subsequent head scans are not required unless clinically indicated. The same radiographic procedures used to assess disease sites at screening should be used for subsequent tumor assessments (e.g., the same contrast protocol for CT scans). Tumor assessments must be continued after disease progression per RECIST v1.1 for patients who receive treatment beyond progression. This includes continued measurement of target lesions, evaluation of non-target lesions (including monitoring for further worsening of any non-target lesions that have shown unequivocal progression), and evaluation of any newly identified lesions (including measurements, if lesions are measurable) at all subsequent assessments.

Overall response at a single timepoint is assessed by the investigator using RECIST v1.1.

### Biomarker Assessments

Biomarker research may include, but not be limited to, analysis of genes or gene signatures associated with tumor molecular subtype and tumor immunobiology, PD-L1, expression of targets specific to each drug combination, EBV, tumor mutation load, MSI status, lymphocyte subpopulations, T cell-receptor repertoire, or cytokines associated with T-cell activation. Research may involve DNA or RNA extraction, analysis of somatic mutations, and use of next-generation sequencing (NGS) (including whole exome sequencing (WES)). Biomarker analyses are performed in an effort to understand the association of these biomarkers with response to study drugs, taking into account efficacy and safety endpoints.

### I. Analysis

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

The analysis results are summarized by the treatment regimen that patients actually receive, as well as by stage (Stage 1 or Stage 2). Data are described and summarized as warranted by sample size. Continuous variables are summarized through use of means, standard deviations, medians, and ranges. Categorical variables are summarized through use of counts and percentages. Listings are used in lieu of tables in the event of small sample sizes.

New baseline values are established for the Stage 2 efficacy and safety analyses. For evaluation of tumor response, new baseline tumor assessments are established. For other endpoints (e.g., change from baseline in vital signs or laboratory test results), the last non-missing value prior to a patient's first dose during Stage 2 serves as the new baseline.

### Determination of Sample Size

This study is not designed to make explicit power and type I error considerations for a hypothesis test. Instead, this study is designed to obtain preliminary efficacy, safety, and PK data on immunotherapy-based treatment combinations when administered to patients with GC or GEJC. Cohort 1 enrolls patients with inoperable, locally advanced, metastatic, or advanced GC or GEJC with adenocarcinoma confirmed as the predominant histology who have not received prior systemic therapy for advanced or metastatic disease.

Approximately 40-90 patients are randomly allocated to the control and experimental arms during the study.

### Primary Efficacy Endpoint

The primary efficacy endpoint is overall response rate (ORR) (defined as the proportion of patients with an objective response (a complete or a partial response)) during Stage 1 (see Table 1). Patients with missing or no response assessments are classified as non-responders.

The ORR is calculated for each arm, along with 90% CIs (Clopper-Pearson method). The difference in ORR between the experimental arms and the control arm is also calculated, along with 90% CIs. CIs are estimated by the exact method or the Wald method, depending on the sample size.

### Secondary Efficacy Endpoints

The secondary efficacy endpoints are PFS, OS, OS at specific timepoints (e.g., 6 months and 12 months), duration of response (DOR), objective response in patients with PD-L1-positive and or TIGIT-positive tumors as assessed by IHC, and disease control during Stage 1 (see Table 1). PFS, DOR, and disease control are determined by the investigator according to RECIST v1.1.

DOR is derived for efficacy-evaluable patients with a complete response or a partial response.

For patients who do not have documented disease progression or die, PFS and DOR is censored at the day of the last tumor assessment.

Patients who are still alive at the time of OS analysis are censored at the last date they were known to be alive.

The Kaplan-Meier method is used to estimate the median for PFS, OS, and DOR, with 90% CIs constructed through use of the Brookmeyer and Crowley method. The OS rate at specific timepoints is also estimated using the Kaplan-Meier method, with 90% CIs calculated based on Greenwood's estimate for variance.

Disease control rate (the proportion of patients with stable disease for ≥ 12 weeks), a partial response, or a complete response, is calculated for each treatment arm, with 90% CIs estimated using the Clopper-Pearson's exact method.

### Safety Analyses

Verbatim adverse event terms are mapped to Medical Dictionary for Regulatory Activities thesaurus terms, and adverse event severity is graded according to NCI CTCAE v5.0 (and also according to the ASTCT CRS Consensus Grading Scale for CRS).

Safety is assessed through summaries of adverse events, changes in laboratory test results, changes in vital signs and ECGs, and exposure to study drugs. Exposure to combination treatment and length of safety follow-up is summarized by treatment arm within each stage.

All verbatim adverse event terms are mapped to Medical Dictionary for Regulatory Activities thesaurus terms, and adverse event severity is graded according to NCI CTCAE v5.0, and severity of CRS is graded by the investigator according to the ASTCT Consensus Grading (Lee et al. Biol Blood Marrow Transplant. 25: 625-638, 2019). All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and causes of death are summarized.

Relevant laboratory, vital sign (pulse rate, respiratory rate, blood pressure, pulse oximetry, and temperature), and ECG data are displayed by time, with grades identified where appropriate. Additionally, a shift table of selected laboratory tests is used to summarize the baseline and maximum post-baseline severity grade. Changes in vital signs and ECGs are summarized.

### Pharmacokinetic Analyses

Sparse samples are collected for potential PK analyses of atezolizumab (patients who receive at least one dose of atezolizumab) and specified drugs given in combination with atezolizumab (patients who receive at least one dose of the drug). Serum or plasma concentrations of the various study drugs may be reported as individual values and summarized (mean, standard deviation, coefficient of variation, median, range, geometric mean, and geometric mean coefficient of variation) by treatment arm, and by cycle and day when appropriate and as data allow. Individual and median serum or plasma concentrations of the various study drugs may be plotted by treatment arm and by cycle and day when appropriate and as data allow. PK data for combination drugs may be compared with available historical data from internal and published previous studies. Atezolizumab or other study drug concentration data may be pooled with data from other studies using an established population PK model to derive PK parameters such as clearance, volume of distribution, and area under the concentration-time curve.

### Immunogenicity Analyses

Immunogenicity may be assessed for atezolizumab and other study treatments as appropriate. The immunogenicity analyses include all patients with at least one anti-drug antibody (ADA) assessment. Patients are grouped according to treatment received or, if no treatment is received prior to study discontinuation, according to treatment assigned.

For atezolizumab, the numbers and proportions of ADA-positive patients and ADA-negative patients at baseline (baseline prevalence) and after baseline (post-baseline incidence) are summarized by treatment group. When determining post-baseline incidence, patients are considered to be ADA positive if they are ADA negative or are missing data at baseline but develop an ADA response following study drug exposure (treatment-induced ADA response), or if they are ADA positive at baseline and the titer of one or more post-baseline samples is at least 0.60-titer units greater than the titer of the baseline sample (treatment-enhanced ADA response).

Patients are considered to be ADA negative if they are ADA negative or are missing data at baseline and all post-baseline samples are negative, or if they are ADA positive at baseline but do not have any post-baseline samples with a titer that is at least 0.60-titer units greater than the titer of the baseline sample (treatment unaffected).

For other study treatments for which ADAs are tested, positivity is determined according to standard methods established for previous studies of these drugs.

The relationship between ADA status and safety, efficacy, PK, and biomarker endpoints may be analyzed and reported using descriptive statistics.

### Interim Analyses

Given the exploratory nature of this study, it is anticipated that interim analyses are conducted during the study, with the earliest (Stage 1) interim analysis taking place when at least one experimental arm has completed enrollment in the preliminary phase and patients have been followed for a minimum of 6 weeks. A posterior probability may be used to guide further enrollment in a treatment arm based on an interim analysis of clinical activity in the experimental arm compared with that in the control arm. If the interim analysis suggests that the activity in an experimental arm is higher than that in the control arm, there may be further enrollment of an additional 25 patients in the experimental arm.

### Example 2: A Phase II, Randomized, Open Label, Parallel-Group Study of Atezolizumab with or without Tiragolumab Following Neoadjuvant Chemoradiotherapy in Patients with Locally Advanced Rectal Cancer

Colorectal cancer (CRC) remains a major cause of cancer deaths worldwide. In China, it is the fifth leading cause of cancer deaths among both men and women and accounted for approximately 8% of all new cancers in 2015 (Chen et al., CA Cancer J Clin. 66: 115-132, 2016), accounting for an estimated 376,300 new cases of colorectal cancer and 191,000 colorectal cancer deaths. The prognosis for patients with metastatic CRC remains poor, with a median 5-year survival of only 12.5% (Siegel et al., CA Cancer J Clin. 64:104-117, 2014). Though the overall incidence and mortality were improved due to the cancer prevention, earlier diagnosis through screening and better treatment modalities, there is still concerning rise in patients presenting with locally advanced disease, especially in rectal cancer (Wolf A et al. 2018). The current standard of care for locally advanced rectal cancer (LARC) is a multimodal approach incorporating neoadjuvant long-course chemoradiotherapy (LCRT) or neoadjuvant hypofractionated short-course radiotherapy (SCRT) followed by total mesorectal excision (TME) and adjuvant fluoropyrimidine-based chemotherapy (Benson et al., J Natl Compr Canc Netw. 18: 806-815, 2020; Yuan et al., Chin J Cancer Res. 31: 423-425, 2019). However, the advancements in rectal cancer treatment have resulted in improvement only in locoregional control and failed to address distant relapse, which is the predominant mode of failure in rectal cancer. The current challenges in the treatment of LARC are two-fold: 1) prolonged of survival by increasing R0 resection rate and reducing distant metastasis; 2) preservation of quality of life in surviving patients by safely avoiding rectal resection or reducing difficulty of surgery.

This example describes a Phase II, randomized, multicenter, open-label, parallel-group study (ML43050) designed to evaluate the efficacy and safety of atezolizumab plus tiragolumab (Atezo+Tira) or atezolizumab alone (Atezo) following standard of care chemoradiotherapy in LARC.

### A. Overview of Study Design

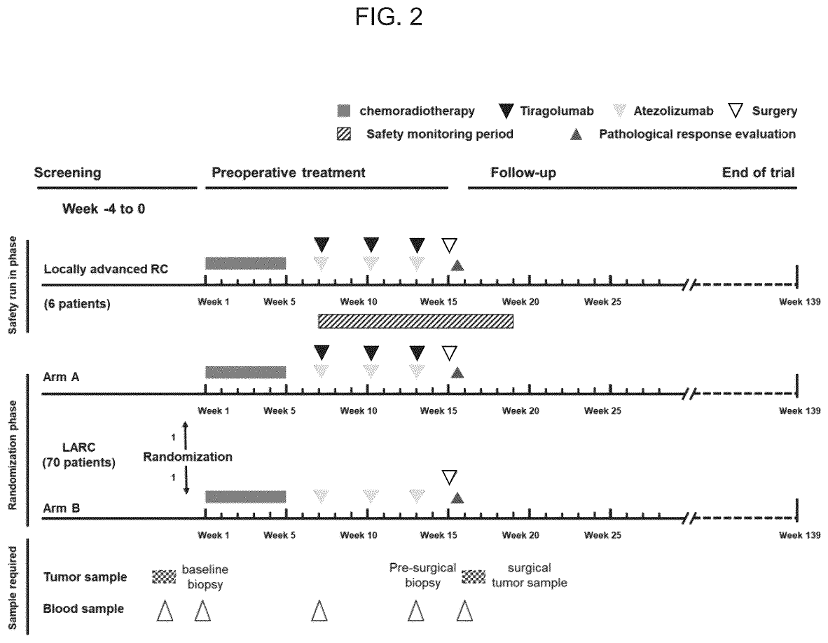

The study evaluates the efficacy and safety of atezolizumab+tiragolumab or atezolizumab alone following neoadjuvant chemoradiotherapy (nCRT) in patients with LARC. Specific objectives and corresponding endpoints for the study are outlined in Table 7. An overview of the study design is presented in FIG. 2.

Herein, “study treatment” refers to nCRT followed by the immunotherapy assigned to patients, meaning either “atezolizumab+tiragolumab” in Arm A or “atezolizumab” in Arm B.

TABLE 7
Objectives and Corresponding Endpoints

| | Corresponding Endpoints |
|---|---|
| Primary Efficacy Objective | |
| To evaluate the efficacy of | Pathological complete response (pCR) rate: defined as the |
| atezolizumab + tiragolumab | proportion of patients achieving pathological complete response in |
| or atezolizumab following | surgery- resected sample evaluated by the local pathologist at each |
| nCRT | site. |
| | pCR is defined as no evidence of vital residual tumor cells on |
| | hematoxylin and eosin evaluation of the complete resected |
| | specimen and all sampled regional lymph nodes following |
| | completion of neoadjuvant therapy (ypT0N0 in the current |
| | AJCC staging system, 8th edition). |
| | pCR rate is analyzed in the intention-to-treat (ITT) population, |
| | defined as all patients who were randomly assigned to the |
| | study treatment, regardless of whether they had surgery. It is |
| | additionally assessed in a modified ITT (mITT) population, |
| | defined as all patients who underwent resection. |
| Secondary Efficacy | |
| Objective | |
| To evaluate the efficacy of | R0 resection rate, defined as the proportion of patients with a |
| atezolizumab + tiragolumab | microscopically margin-negative resection, in which no gross or |
| or atezolizumab following | microscopic tumor remains in the primary tumor bed and/or sampled |
| nCRT | regional lymph nodes based on evaluation by the local pathologist. |
| | Objective response rate (ORR) before surgery, defined as the |
| | proportion of patients with a complete response (CR) or partial |
| | response (PR), as determined by the investigator according to |
| | RECIST v1.1. |
| | Complete clinical response (cCR) rate, defined as the |
| | proportion of patients with no local evidence of residual tumor |
| | (yield clinical T0N0, ycT0N0) after neoadjuvant |
| | chemoradiotherapy assessed by endoscopy MRI and phycial |
| | examination. |
| | One/two/three-year relapse-free survival (1/2/3y-RFS) rate, defined |
| | as the proportion of patients who have not experienced disease |
| | relapse or death from any cause at 1/2/3 year(s), as determined by |
| | the investigator. |
| | One/two/three-year event free survival (1/2/3- EFS) rate, defined as |
| | the proportion of patients who have not experienced the following |
| | events at 1/2/3 year(s) from randomization: progression of disease |
| | that precludes surgery, local or distant recurrence, or death due to |
| | any cause. |
| | Any recurrence of the original cancer, appearance of a new RC |
| | or distant metastasis should be proven by cytology or histology |
| | when possible. An isolated event of increased tumor markers, |
| | or unexplainable clinical deterioration is not considered to be |
| | evidence of relapse without support of other objective |
| | measurements (e.g., radiology, histology/cytology). Date of |
| | relapse is defined as the date of definitive assessment by |
| | objective measurements. |
| | pCR rate, DFS/EFS is additionally assessed in subgroup population |
| | with PD-L1/PVR/TIGIT positive expression in baseline, pre-surgery |
| | and surgery tumor tissue |
| | PD-L1 expression. Expression of PD-L1 is assessed in pre- |
| | treatment, pre-surgery and surgery tumor samples by the |
| | immunohistochemistry assay (investigational Ventana PD-L1 |
| | (SP263) Companion Diagnostic (CDx) Assay) in the central |
| | laboratory. Tumors are considered PD-L1 positive if the tumor |
| | and tumor-associated immune cell (TIC) score is 5% or greater. |
| | PVR expression. Expression of PVR is assessed in pre- |
| | treatment, pre-surgery and surgery tumor samples by the |
| | immunohistochemistry assay in the central laboratory. |
| | TIGIT expression. Expression of TIGIT is assessed pre- |
| | treatment, pre-surgery and surgery tumor samples by the |
| | immunohistochemistry assay (investigational Ventana TIGIT |
| | Companion Diagnostic (CDx) Assay) in the central laboratory. |
| Safety Objective | |
| To evaluate the safety of | Incidence and severity of adverse events, with severity determined |
| atezolizumab or | according to NCI CTCAE v5.0. |
| atezolizumab + tiragolumab | Severity of cytokine-release syndrome (CRS) is determined |
| following nCRT | according to the American Society for Transplantation and |
| | Cellular Therapy (ASTCT) CRS Consensus Grading scale. |

| | Corresponding Endpoints |
|---|---|
| Biomarker Objective | (performed with permission from HGRAC) |
| To identify and/or evaluate | Tumor-infiltrating lymphocytes (TILs) |
| biomarkers that are | TILs signature is assessed by multiplex immunohistochemistry |
| associated with progression | (mIHC) staining on pre-treatment, pre-surgery and surgery |
| to a more severe disease | tumor samples in the central laboratory. Briefly, 4-5 μm |
| state (i.e., prognostic | sections of FFPE are applied with several antibodies (FoxP3, |
| biomarkers), are associated | CD56, CD8, CD4, Granzyme B, CD155, PD-L1, TIGIT) to |
| with acquired resistance to | evaluate the subpopulation and function of different kinds of |
| study treatment, can provide | lymphocytes. Further analysis such as total numbers of TILs, |
| evidence of study treatment | numbers and/or density of specific type of cells, correlation of |
| activity (i.e., predictive | different markers, changes comparison between baseline and |
| biomarkers), or can increase | pre-surgery samples and their association with progression is |
| the knowledge and | conducted to identify and/or evaluate potential biomarkers or |
| understanding of disease | deepen the understanding of disease biology and drug safety. |
| biology and drug safety | Circulating tumor DNA (ctDNA) |
| | Blood samples are collected at several time points. ctDNA is |
| | extracted from the whole blood samples and analyzed in the |
| | central laboratory with a pan-cancer panel. |

Atezo + Tira = atezolizumab plus tiragolumab; nCRT = neoadjuvant chemoradiotherapy; pCR = pathological complete response; NCI CTCAE v5.0 = National Cancer Institute Common Terminology Criteria for Adverse Events, Version 5.0; RESICT v1.1 = Response Evaluation Criteria in Solid Tumor, Version 1.1. HGRAC = Human Genetic Resources Administration of China.

The study is conducted in two phases and enrolls approximately 76 patients.
Safety run-in phase: approximately 3-6 patients receive atezolizumab+tiragolumab under a “3+3” design.
Randomization phase: patients are randomized in a 1:1 ratio to the atezolizumab+tiragolumab arm (Arm A) or atezolizumab arm (Arm B).

After the safety run-in phase, a total of 70 patients are enrolled and randomized in this study (i.e., 35 patients in each arm). All patients are enrolled in China. Eligible patients receive one of the treatments as outlined below (Table 8).

TABLE 8
Study Design

| | Treatment | nCRT | Sequential immunotherapy* |
|---|---|---|---|
| Study phase | arm | Week 1-5 | Week 8-14 |
| Safety run-in | / | RT: 45-50.4 Gy in 25-28 | Atezolizumab 1200 mg IV plus |
| | | fractions to the pelvis on Day | tiragolumab 600 mg IV on Day 1 |
| | | 1-5 every week | of each 21-day cycle for 3 cycles. |
| | | CT: Capecitabine 825 mg/m² | |
| | | P.O. Bid or 5-fluorouracil (5- | |
| | | FU) 225 mg/m² IV over 24 | |
| | | hours 5 days/week during | |
| | | RT. | |
| Randomization | A | RT: 45-50.4 Gy in 25-28 | Atezolizumab 1200 mg IV plus |
| | | fractions to the pelvis on Day | tiragolumab 600 mg IV on Day 1 |
| | | 1-5 every week | of each 21-day cycle for 3 cycles. |
| | B | CT: Capecitabine 25 mg/m² | Atezolizumab 1200 mg IV on Day |
| | | P.O. BID or 5-FU 225 mg/m² | 1 of each 21-day cycle for 3 |
| | | IV over 24 hours 5 days/week | cycles. |
| | | during RT. | |

RT = radiotherapy; CT = chemotherapy.
*Patients are assessed after nCRT by the opinion of investigator, and those eligible for immunotherapy receive Atezo + Tira or Atezo.

### Safety Run-In Phase

Patients are first accrued into the safety run-in phase. Patients enrolled in the safety run-in phase receive Atezo+Tira following nCRT. Upon determination of the safety of the treatment regimen, the study proceeds to the randomization phase.

The first 3 patients are enrolled in the safety run-in phase consecutively. An overview of the study schedule is presented in FIG. 2. After signing the informed consent form, patients undergo screening procedures that include laboratory tests (e.g., hematology, chemistries, liver function tests); contrast-enhanced CT scan, or MRI of the chest, abdomen, pelvis and head; assessments (including tumor, laboratory, biomarker assessments, and endoscopy); and tumor biopsies as in the randomization phase.

Study treatment in the safety run-in phase comprises 5-FU or capecitabine-based chemoradiotherapy, followed by atezolizumab at a dose of 1200 mg combined with tiragolumab at a dose of 600 mg, on a Q3W schedule for 3 cycles (Table 8, Safety Run-in).

The safety run-in phase follows a “3+3” approach. The first 3 patients enrolled in this phase are closely monitored for all safety events until 30 days after surgery.
Any occurrence of the following events trigger an immediate review of all the relevant safety data:
   Any of the three patients experiences death of any cause from the beginning of study treatment to 30 days post-surgery.
   Any of the three patients experiences delays or cancellation of surgery of any cause.
   During the immediate review, the treatment and enrollment of the other patients in this phase continues as planned.
All safety data of the first 3 patients is reviewed at 30 days after the last of the 3 patients' finishing surgery. Based on review of these safety data, it is decided whether:
   An additional 3 patients should be enrolled, or
   Additional 3 patients are not required for the safety run-in phase, and the randomization phase should be opened, or
   The study should be suspended.
If an additional 3 patients are enrolled in the safety run-in phase, the same review process is followed. The safety data of all the 6 patients is reviewed before final decision is made as to whether:
   The randomization phase should be opened, or
   The study should be suspended.

During the safety run-in phase, if a patient does not receive at least one complete cycle of Atezo combined with Tira after nCRT, this patient is replaced.

### Randomization Phase

This phase enrolls approximately 70 patients, who are randomized in a 1:1 ratio to the Atezo+Tira arm or the Atezo arm. Eligible patients receive one of the treatments as described in Table 8. FIG. 2 presents an overview of the study design.

Patients receiving nCRT treatment should have recovered from the treatment prior to sequential immunotherapy, and must meet the following criteria:
ANC≥1.5×10⁹/L (1500/μL) without granulocyte colony-stimulating factor support.
Lymphocyte count ≥0.5×10⁹/L (500/μL).
Platelet count ≥100×10⁹/L (100,000/μL) without transfusion.
Hemoglobin ≥90 g/L (9 g/dL).

Atezo+Tira or Atezo is administered at least 2 weeks after the completion of nCRT.

Administration can be suspended for a period up to 4 weeks before all criteria are met. Administration should be resumed as soon as possible after the criteria above are met.

All patients eligible for curative surgery undergo such surgery. It is recommended that surgery be performed 2 weeks (+1 week) after the last dose of Atezo+Tira or Atezo; patients experiencing adverse events after immunotherapy should undergo surgery as soon as they have recovered from the adverse events within 4 weeks after the last dose of Atezo+Tira or Atezo. Radical surgical resection using total mesorectal excision (TME) and lymph node dissection is tailored to the individual patient with the goal of achieving R0 resection.

Adjuvant treatment after surgery is decided by the investigator according to the local standard of care.

Patients for whom atezolizumab is transiently withheld or permanently discontinued may not continue on tiragolumab as a single agent or modified dose. Patients for whom tiragolumab is transiently withheld or permanently discontinued may continue on atezolizumab single-agent therapy as long as the patients are experiencing clinical benefit in the opinion of investigator. No dose modification for either atezolizumab or tiragolumab is allowed. On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, immune-mediated adverse events should generally be attributed to both agents, and dose interruptions or treatment discontinuation in response to immune-mediated adverse events should be applied to both tiragolumab and atezolizumab.

Patients who discontinue neoadjuvant therapy prematurely as a result of disease progression or who receive non-protocol therapy prior to surgery must be discontinued from all study treatment, and are managed per local practice. These patients remain on study for survival follow-up. Patients who discontinue neoadjuvant therapy due to toxicity and patients who were not eligible for surgery are subsequently treated according to local practice.

Patients undergo tumor assessments at scheduled intervals during the study.

Tumor specimen acquired from surgery is collected for pCR evaluation and tested for pathological response by experienced pathologists at each site. Tumor tissue (via biopsies and/or surgical resection) and blood samples from eligible patients are provided to a central laboratory, and are prospectively tested and analyzed for biomarkers that might be associated with clinical benefit, tumor immunobiology, mechanisms of resistance, etc.

Patients are closely monitored for adverse events throughout the study, including the incidence, nature and severity of adverse events and laboratory abnormalities graded per National Cancer Institute Common Terminology Criteria for Adverse Events, Version 5.0 (NCI CTCAE 5.0).

All available safety data from randomization to 30 days after last dose of study treatment of the first 20 randomized patients is reviewed by the IMC in order to provide a recommendation as to whether to continue, modify or terminate the study.

Following treatment completion or discontinuation, follow-up information is collected by telephone, patient medical records, and/or clinic visits every 6 months until 3 years after surgery, death, loss to follow-up, withdrawal of informed consent, or study termination by the Sponsor, whichever occurs first.

During the randomization phase, patients who withdraw from the study are not replaced.

### B. End of Study and Length of Study

The end of this study is defined as the date when the last patient, last visit (LPLV) occurs or safety follow-up is received from the last patient, whichever occurs later. The end of the study is expected to occur 40 months after the last patient is enrolled.

The total length of the study, from enrollment of the first patient to the end of the study, is expected to be approximately 60 months.

### C. Rationale for Study Design and Patient Population

In this study, tiragolumab and atezolizumab are administered following nCRT in LARC patients. Although tiragolumab and atezolizumab have been administered in CRC patients previously and there is also an ongoing study showing safety and tolerability of immunotherapy in LARC (as there might be some potential overlapping toxicities between chemoradiotherapy and immunotherapy), the study starts with a safety run-in phase under a “3+3” design to closely monitor safety and tolerability as well as to better understand possible risks of the treatment setting. A “3+3” design both limits the number of patients exposed to this new combination and collects comprehensive safety data on tiragolumab and atezolizumab following nCRT.

This study enrolls patients with LARC, regardless of PD-L1 expression level in the tumor. This target population has been chosen to address their unsatisfactory outcomes and need for improved treatment strategy. Despite the improvement in treatment of LARC, the long-term prognosis is still dismal, with nearly one-third of patients dying from distant metastasis (Cunningham et al. Lancet. 375: 1030-1047, 2010). Furthermore, most survivors, even those achieving clinical complete response after nCRT, experience rectum removal surgery due to the unsatisfactory depth of response, which significantly impairs their quality of life (Pucciarelli et al. Ann Surg. 253: 71-77, 2011). Therefore, there is a continuing need for more efficacious, better tolerated treatments for patients with LARC.

Tumor-cell killing by cytotoxic chemotherapy may expose the immune system to high levels of tumor antigens. Boosting tumor-specific T-cell immunity in this setting by blocking the PD-L1 pathway may result in deeper and more durable responses than those observed with standard chemoradiotherapy alone (Merritt et al. J Thorac Cardiovasc Surg. 126: 1609-1617, 2003; Apetoh et al. Nat Med. 13: 1050-1059, 2007), and this may reasonably occur in tumors regardless of PD-L1 expression.

### D. Inclusion Criteria

Approximately 76 patients with rectal cancer disease are enrolled in this study. Patients must meet the following criteria for study entry:
Age ≥18 years at time of signing Informed Consent Form.
Histologically or cytologically confirmed diagnosis of adenocarcinoma of the rectum. Microsatellite instability (MSI) or mismatch-repair (MMR) status should be documented, and patients with unknown MSI status are required to undergo testing at a local laboratory and provide results at screening
Resectable locally advanced rectal cancer, with clinical stage as cT₃N+M₀ or cT₄N_{any}M₀ per AJCC/UICC 8th edition.
The inferior margin of the tumor ≤10 cm from the anal verge.
No prior anti-cancer treatment (including both local-regional and systemic treatment) for rectal cancer.
Availability of a representative tumor specimen that is suitable for pathological evaluation and biomarker expression analysis.
   A formalin-fixed, paraffin-embedded (FFPE) tumor specimen in a paraffin block (preferred) or approximately 12-15 slides containing unstained, freshly cut, serial sections should be submitted along with an associated pathology report within 4 weeks of randomization.
   If archival tumor tissue is unavailable or is determined to be unsuitable for required testing, tumor tissue must be obtained from a biopsy performed at screening.
   If fewer than 12 slides are obtained, but there are enough slides for biomarker analysis by central lab, patients are still eligible.
ECOG Performance status (PS) of 0 or 1 within 7 days prior to randomization.
At least one measurable lesion per RESIST v1.1.
Adequate hematologic and end-organ function, defined by the following laboratory test results, obtained within 7 days prior to initiation of study treatment:
   ANC≥1.5×10⁹/L (1500/μL) without granulocyte colony-stimulating factor support.
   Lymphocyte count ≥0.5×10⁹/L (500/μL).
   Platelet count ≥100×10⁹/L (100,000/μL) without transfusion.
   Hemoglobin ≥90 g/L (9 g/dL).
      Patients may be transfused to meet this criterion, but must not have been transfused within 2 weeks prior to screening.
   AST, ALT, and alkaline phosphatase (ALP)≤2.5× upper limit of normal (ULN).
   Total bilirubin ≤1.5×ULN with the following exception:
      Patients with known Gilbert disease: total bilirubin ≤3×ULN.
   Serum creatinine ≤1.5×ULN or Creatinine clearance ≥50 mL/min (calculated using the Cockcroft-Gault formula).
   Albumin ≥25 g/L (2.5 g/dL).
   For patients not receiving therapeutic anticoagulation: INR and aPTT≤1.5×ULN.
For patients receiving therapeutic anticoagulation: stable anticoagulant regimen.
Negative HIV test at screening.
For women of childbearing potential: agreement to remain abstinent (refrain from heterosexual intercourse) or use contraception, and agreement to refrain from donating eggs.
For men: agreement to remain abstinent (refrain from heterosexual intercourse) or use contraceptive methods and agreement to refrain from donating sperm.

### E. Exclusion Criteria

Patients who meet any of the following criteria are excluded from study entry:
Evidence of metastatic disease.
Histology consistent with small cell carcinoma, squamous carcinoma, or mixed carcinoma.
Presence of synchronous colorectal cancer.
Patients with obstruction or imminent obstruction, presence but not limited the symptom of stopping flatus and defecation.
   Patients with incomplete obstruction and tolerant of nCRT treatment are eligible for enrollment.
Clinical symptoms or radiological suspicion of bowel perforation.
Patients not eligible for long-course radiotherapy.
History of malignancies other than rectal cancer within 3 years prior to screening with the exception of those with a negligible risk of metastasis or death (e.g., expected 5-year overall survival >90%) treated with expected curative outcome, such as adequately treated carcinoma in situ of the cervix, non-melanoma skin carcinoma, localized prostate cancer, ducal carcinoma in situ, or stage I uterine cancer.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, multiple sclerosis, vasculitis or glomerulonephritis, with the following exceptions:
   Patients with a history of autoimmune-related hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met:
      Rash must cover <10% of body surface area.
      Disease is well controlled at baseline and requires only low-potency topical corticosteroids
      No occurrence of acute exacerbations of the underlying condition requiring psoralen plus ultraviolet A radiation, methotrexate, retinoid, biologic agents, oral calcineurin inhibitors, or high-potency or oral corticosteroids within the previous 12 months.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
   Patients with known coronary artery disease, congestive heart failure not meeting the above criteria, or left ventricular ejection fraction <50% must be on a stable medical regimen that is optimized in the opinion of the treating physician, in consultation with a cardiologist if appropriate.
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan.
   History of radiation pneumonitis in the radiation field (fibrosis) is permitted.
Severe chronic or active infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to initiation of study treatment
   Patients receiving prophylactic antibiotics (e.g., to prevent a urinary tract infection or chronic obstructive pulmonary disease exacerbation) are eligible for the study.
Active tuberculosis.
Positive EBV viral capsid antigen IgM test at screening.
   An EBV polymerase chain reaction (PCR) test should be performed as clinically indicated to screen for acute infection or suspected chronic active infection. Patients with a positive EBV PCR test are excluded.
Patients with active hepatitis B (chronic or acute; defined as having a positive hepatitis B surface antigen [HBsAg] test at screening).
   Patients with past hepatitis B virus (HBV) infection (defined as the presence of hepatitis B core antibody [anti-HBc] and absence of HBsAg) are eligible if HBV DNA is not detectable prior to C1D1.
Patients with active hepatitis C. Patients positive for hepatitis C virus (HCV) antibody are eligible only if PCR is negative for HCV RNA.
Uncontrolled tumor-related pain.
   Patients requiring pain medication must be on a stable regimen at study entry.
Uncontrolled pleural effusion, pericardial effusion, or ascites requiring recurrent drainage procedures (once monthly or more frequently).
   Patients with indwelling catheters (e.g., PLEURX®) are allowed.
Uncontrolled or symptomatic hypercalcemia (ionized calcium >1.5 mmol/L, calcium >12 mg/dl, or corrected calcium >ULN).
Uncontrolled diabetes or Grade ≥2 abnormalities in potassium, sodium, despite standard medical management within 14 days prior to initiation of study treatment.
History of leptomeningeal disease.
Prior treatment with CD137 agonists, T-cell co-stimulating, or immune checkpoint blockade therapies, including anti-CTLA-4, anti-PD-1, anti-PD-L1 and anti-TIGIT therapeutic antibodies.
Treatment with systemic immunostimulatory agents (including, but not limited to, interferon and IL-2) within 4 weeks or 5 half-lives of the drug (whichever is longer) prior to initiation of study treatment
Treatment with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) may be eligible for the study after discussion with the Medical Monitor.
   Patients who received mineralocorticoids (e.g., fludrocortisone), inhaled or low-dose corticosteroids for chronic obstructive pulmonary disease (COPD) or asthma, or low-dose corticosteroids for orthostatic hypotension or adrenal insufficiency are eligible for the study.
Prior allogeneic stem cell or solid organ transplantation.
Treatment with a live, attenuated vaccine within 4 weeks prior to initiation of study treatment, or anticipation of need for such a vaccine during study treatment or within 5 months after the final dose of study treatment.
Major surgical procedure or significant traumatic injury within 28 days prior to initiation of study treatment, or abdominal surgery, abdominal interventions or significant abdominal traumatic injury within 60 days prior to initiation of study treatment, or anticipation of need for major surgical procedure during the course of the study, or non-recovery from side effects of any such procedure.
Any other disease, medical condition or abnormality, metabolic dysfunction, alcohol or drug abuse or dependence, physical examination finding, clinical laboratory finding that contraindicates the use of an investigational drug, may affect the interpretation of the results, or may render the patient at high risk from treatment complications.
Treatment with any other investigational agent with therapeutic intent within 28 days prior to study treatment initiation.
History of severe allergic anaphylactic reactions to chimeric or humanized antibodies or fusion proteins.
Known hypersensitivity to Chinese hamster ovary cell products or to any component of the atezolizumab or tiragolumab formulation.
History of allergic reactions to chemotherapy drugs (5-FU and capecitabine).
Known dihydropyrimidine dehydrogenase (DPD) deficiency or history of severe and unexpected reactions to fluoropyrimidine therapy in patients selected to receive capecitabine.
Pregnant or breastfeeding, or intending to become pregnant during the study or within 5 months after the final dose of atezolizumab or 90 days after the final dose of tiragolumab, or 6 months after the final dose of capecitabine/5-FU.
   Women of childbearing potential must have a negative serum pregnancy test result within 14 days prior to initiation of study treatment.

### Study Treatment

The investigational medicinal products (IMPs) for this study are atezolizumab and tiragolumab. Chemotherapy drugs (5-FU or capecitabine) are administered as concomitant medication with radiation, in which they are considered to be non-investigational medicinal products (NIMPs). Patients receive study treatment until they finish the study treatment cycles or experience unacceptable toxicity or loss of benefit as determined by the investigator after an integrated assessment of radiographic and biochemical data, local biopsy results (if available), and clinical status.

Patients are randomly assigned to one of two treatment arms: Arm A (Atezo+Tira) or Arm B (Atezo). Randomization occurs in a 1:1 ratio through use of a permuted-block randomization method to ensure a balanced assignment to each treatment arm.

### Atezolizumab

Atezolizumab is administered by IV infusion at a fixed dose of 1200 mg on Day 1 of each 21-day cycle until unacceptable toxicity or loss of clinical benefit as determined by the investigator after an integrated assessment of radiographic and biochemical data, local biopsy results (if available), and clinical status.

Administration of atezolizumab is performed in a monitored setting where there is immediate access to trained personnel and adequate equipment and medicine to manage potentially serious reactions. Atezolizumab infusions are administered per the instructions outlined in Table 9.

No dose modification for atezolizumab is allowed.

TABLE 9
Administration of first and subsequent atezolizumab infusions

| | First infusion | Subsequent infusions |
|---|---|---|
| Atezolizumab | No premedication is permitted prior | If the patient experienced an infusion- |
| infusion | to the atezolizumab infusion. | related reaction with any previous |
| | Vital signs (pulse rate, respiratory | infusion, premedication with |
| | rate, blood pressure, and | antihistamines, antipyretics, and/or |
| | temperature) should be measured | analgesics may be administered for |
| | within 60 minutes prior to the | subsequent doses at the discretion of |
| | infusion. | the investigator. |
| | Atezolizumab should be infused over | Vital signs should be measured within |
| | 60 (±15) minutes. | 60 minutes prior to the infusion. |
| | If clinically indicated, vital signs | Atezolizumab should be infused over |
| | should be measured every 15 (±5) | 30 (±10) minutes if the previous infusion |
| | minutes | was tolerated without an infusion- |
| | | related reaction, or 60 (±15) minutes if |
| | | the patient experienced an infusion- |
| | | related reaction with the previous |
| | | infusion. |
| | | If clinically indicated, vital signs should |
| | | be recorded during the infusion |
| Observation | After the infusion of atezolizumab, | If the patient tolerated the previous |
| period after | the patient begins a 60-minute | atezolizumab infusion well without |
| atezolizumab | observation period. | infusion-associated adverse events, the |
| infusion | Vital signs should be recorded at 30 | observation period after the next and |
| | (±10) minutes after the infusion of | following infusions may be reduced to |
| | atezolizumab. | 30 minutes. |
| | Patients should be informed about | If the patient experienced infusion- |
| | the possibility of delayed post- | associated adverse events in the |
| | infusion symptoms and instructed to | previous infusion, the observation |
| | contact their study physician if they | period should be 60 minutes. |
| | develop such symptoms. | If clinically indicated, vital signs should |
| | | be recorded at 30 (±10) minutes after |
| | | the infusion of atezolizumab. |

### Tiragolumab

Tiragolumab is administered by IV infusion at a fixed dose of 600 mg on Day 1 of each 21-day cycle. On Day 1 of cycle 1, tiragolumab is administered 60 minutes after completion of the atezolizumab infusion. The interval between tiragolumab and atezolizumab is 30 minutes if the previous atezolizumab infusion was tolerated without an IRR or 60 minutes if the patient experienced an IRR with the previous atezolizumab infusion. Tiragolumab infusions are administered per the instructions outlined in Table 10.

No dose modification for tiragolumab is allowed.

TABLE 10
Administration of first and subsequent tiragolumab infusions

| | | Day 1 Infusion of Subsequent |
|---|---|---|
| | Day 1, Cycle 1 Infusion | Cycles |
| Infusion of | No premedication is permitted prior | If the patient experienced an IRR |
| tiragolumab | to the tiragolumab infusion. | during any previous infusion of |
| | Vital signs (pulse rate, respiratory | tiragolumab, premedication with |
| | rate, blood pressure, and | an antihistamine and/or antipyretic |
| | temperature) should be recorded | may be administered for |
| | within 60 minutes prior to the | subsequent doses, at the |
| | infusion. | discretion of the investigator. |
| | Tiragolumab should be infused over | Vital signs should be recorded |
| | 60 (±10) minutes. | within 60 minutes prior to the |
| | Vital signs should be recorded every | infusion. |
| | 15 (±5) minutes during the infusion | Tiragolumab should be infused |
| | | over 30 (±10) minutes if the |
| | | previous infusion was tolerated |
| | | without an IRR, or 60 (±10) |
| | | minutes if the patient experienced |
| | | an IRR with the previous infusion. |
| | | Vital signs should be recorded |
| | | during the infusion if clinically |
| | | indicated. |
| Observation | After the infusion of tiragolumab, the | If the patient tolerated the previous |
| period after | patient begins a 60-minute | infusion of tiragolumab well |
| infusion of | observation period. | without infusion-associated |
| tiragolumab | Vital signs should be recorded at 30 | adverse events, the observation |
| | (±10) minutes after the infusion of | period may be reduced to 30 |
| | tiragolumab. | minutes. |
| | Patients are informed about the | If the patient experienced an |
| | possibility of delayed post-infusion | infusion-associated adverse event |
| | symptoms and are instructed to | in the previous infusion, the |
| | contact their study physician if they | observation period should be 60 |
| | develop such symptoms. | minutes. |
| | | If clinically indicated, vital signs |
| | | should be recorded at 30 (±10) |
| | | minutes after the infusion of |
| | | tiragolumab. |
| | | Patients are informed about the |
| | | possibility of delayed post-infusion |
| | | symptoms and are instructed to |
| | | contact their study physician if |
| | | they develop such symptoms. |

IRR = infusion-related reaction.

### 5-FU and Capecitabine

5-FU and capecitabine are the chemotherapy agents of this study. Patients are assigned to receive either 5-FU or capecitabine concurrently with radiotherapy based on the decision of the investigator according to local clinical practice.

5-FU is intravenously administered on 5 or 7 consecutive days during the radiotherapy according to the following guidelines:
The appropriate daily dose of capecitabine is in accordance with body surface area (BSA). 5-FU at 225 mg/m² is administered by continuous IV infusion, 5 or 7 days a week during the first five weeks of study treatment.

Capecitabine is orally administered 5 or 7 days/week concurrently with radiotherapy during the first five weeks of study treatment according to the following:
The appropriate daily dose of capecitabine is in accordance with the body surface area (BSA). Capecitabine at 825 mg/m² is orally administered twice daily, 5 or 7 days a week during first five weeks of study treatment.
Capecitabine is administered orally within 30 minutes after the end of a meal (breakfast, dinner). Tablets should be swallowed with approximately 200 ml water (not fruit juice).
The capecitabine dose is the combined dose of 500 mg tablets. The total daily dose is divided into two amounts and given roughly 12 hours apart. Two doses may be separated in order to take a whole tablet.

The choice of administering capecitabine on 5 days or 7 days/week and 5-FU on 5 or 7 days/week depends on the investigator's choice and/or local management guidelines. The dose of chemotherapy is calculated according to the patient's body surface area (BSA). The BSA and the amount of drug administered must be recalculated if the patient's body weight has changed by >10% (increased or decreased) from baseline. Recalculation of the amount of drug administered on the basis of smaller changes in body weight or BSA is at the investigators' discretion.

Dose modification for 5-FU or capecitabine is allowed.

### Radiotherapy

The total irradiation dose of 45-50.4 Gy is delivered in 25-28 fractions, with a daily fraction of 1.8 Gy over the first five weeks excluding weekends, concurrently with fluoropyrimidine-based chemotherapy.

It is recommended that the radiation therapy fields include the tumor or tumor bed, with a 2-5 cm margin, and the mesorectum, the presacral nodes, and the internal iliac nodes. The external iliac nodes should also be included for T4 tumors involving anterior structures.

Multiple radiation fields should be used. Positioning and other techniques to minimize the volume of small bowel in the fields is encouraged.

### F. Supportive Medication

Supportive medications (anti-emetics, antihistamines, and analgesics) are administered per local practice standards. Because of the immunomodulatory effects of corticosteroids, premedication with corticosteroids should be minimized to the extent that is clinically feasible.

No premedication is indicated for the administration of Cycle 1 of atezolizumab and tiragolumab. However, patients who experience an IRR with Cycle 1 of atezolizumab and/or tiragolumab may receive premedication with antihistamines, antipyretics, and/or analgesics (e.g., acetaminophen) for subsequent infusions. Metamizole (dipyrone) is prohibited in treating IRRs because of its potential for causing agranulocytosis.

### G. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the treatment discontinuation visit.

### Permitted Therapy

Patients are permitted to use the following therapies during the study:
Oral contraceptives with a failure rate of <1% per year.
Hormone-replacement therapy.
Prophylactic or therapeutic anticoagulation therapy (such as warfarin at a stable dose or low-molecular-weight heparin); international normalized ratio (INR) should be intensively monitored during anticoagulation therapy.
Inactivated influenza vaccinations.
Megestrol acetate administered as an appetite stimulant.
Mineralocorticoids (e.g., fludrocortisone).
Corticosteroids administered for chronic obstructive pulmonary disease or asthma.
Low-dose corticosteroids administered for orthostatic hypotension or adrenocortical insufficiency.
Premedication with antihistamines, antipyretics, and/or analgesics may be administered for the second and subsequent atezolizumab infusions only, at the discretion of the investigator.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H₂-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and ß2-adrenergic agonists).

### H. Assessments

Patients are closely monitored for safety and tolerability throughout the study. Patients should be assessed for toxicity prior to each dose; dosing occurs only if the clinical assessment and local laboratory test values are acceptable.

### Tumor and Response Evaluations

Patients undergo tumor assessments at baseline, 8 weeks after nCRT completion, and after the last dose of Atezo+Tira or Atezo prior to surgery, every 6 months after surgery, regardless of dose delays, until radiographic disease progression per RECIST v1.1 or until disease recurrence, withdrawal of informed consent, death, or the study is terminated by the Sponsor, whichever occurs first. Thus, tumor assessments continue according to the schedule in patients who discontinue treatment for reasons other than disease progression, even if they start new anti-cancer therapy. Tumor assessments may be repeated at any time if progressive disease is suspected.

All measurable and evaluable lesions should be assessed and documented at screening. Tumor assessments performed as standard of care prior to obtaining informed consent and within 28 days prior to initiation of study treatment do not have to be repeated at screening.

All measurable and evaluable lesions identified at baseline should be re-assessed at each subsequent tumor evaluation. The same radiographic procedures used to assess disease sites at screening should be used for subsequent tumor assessments (e.g., the same contrast protocol for CT scans).

Objective response at a single timepoint is determined by the investigator according to RECIST v1.1.

### Biomarker Assessments

MSI or MMR status of the tumor at baseline samples.
Archival or newly collected tumor tissue sample obtained at baseline for efficacy objective analysis and exploratory research on biomarkers.
Biopsy tumor tissue sample obtained prior to the surgery for efficacy objective analysis and exploratory research on biomarkers.
Tumor tissue sample obtained at the surgery for efficacy objective analysis and exploratory research on biomarkers.

ctDNA from blood samples for exploratory research on biomarkers should be collected before, during and at discontinuation of study treatment, additional collection could be performed during the follow up phase if clinically necessary determined by the investigator.

Exploratory biomarker research may include analysis of protein and/or genic signatures associated with tumor immunobiology. Tumor-infiltrating lymphocytes (TILs) signature is assessed by multiplex immunohistochemistry (mIHC) staining on pre-treatment, pre-surgery and surgery tumor samples in the central laboratory. Briefly, several antibodies (FoxP3, CD56, CD8, CD4, Granzyme B, CD155, PD-L1, TIGIT) are applied to 4-5 μm FFPE sections to evaluate the subpopulation and function of different kinds of lymphocytes. Further analysis such as total numbers of TILs, numbers and/or density of specific type of cells, correlation of different markers, changes comparison between baseline and pre-surgery samples and their association with progression are conducted to identify and or evaluate potential biomarkers or deep the understanding of disease biology and drug safety. Blood samples are collected at several time points. ctDNA is extracted from the whole blood samples and analyzed in the central laboratory with a pan-cancer panel. Guided by clinical and nonclinical data, additional exploratory research to evaluate tumor pharmacodynamic biomarkers and potential predictive biomarkers to understand the mechanism of action of the drug, and of disease biology using assays for analysis of proteins and/or genes may be performed.

### I. Analysis

The statistical considerations and analysis plan are summarized below. The analysis set that is used in this study is defined as below (Table 11).

TABLE 11
Description of analysis sets

| Analysis Set | Description |
|---|---|
| Intention-to- | All patients who are randomly assigned to the study |
| Treat (ITT) | treatment, regardless of whether they had surgery. Analyses |
| | performed on the ITT population consider patients' |
| | allocation to study treatment groups as randomized. |
| Modified | A subset of ITT, which includes all randomized patients |
| ITT | who underwent resection. |
| (mITT) | |
| Safety | All randomized patients who received at least one dose of |
| | study treatment. Analyses consider patients as treated. |

### Determination of Sample Size

The purpose of this study is estimation and hypothesis generation regarding the effect of Atezo+Tira or Atezo following nCRT on the pathological complete response (pCR) rate relative to preoperative chemoradiotherapy according to historical data. Point and interval estimates of pCR rate are obtained. A total of 70 patients are randomized in this study. The sample size calculation is based on following assumptions:
15% pCR rate for preoperative chemoradiotherapy according to historical data.
35% pCR rate for Atezo+Tira (or Atezo) following nCRT.
1:1 randomization.
Alpha=0.05 (Two-sided).
Power=80%.

Based on these considerations, a total of 31 patients need to be enrolled in each arm of Atezo+Tira or Atezo to demonstrate efficacy of pCR compared with preoperative chemoradiotherapy (15% pCR rate according to historical data). Taking into consideration a 10% drop-out rate, 35 patients are needed for each arm and 70 patients need to be randomized for the study. With an additional 3 or 6 patients enrolled for the safety run-in phase, a total of 73 or 76 patients are enrolled in the study.

The sample size is calculated using “Test for one proportion” in PASS version 16.0.4.

### Efficacy Analysis

### Primary Efficacy Endpoint

No comparisons between the two arms are performed on the primary endpoint. The ITT analysis set serves as the primary analysis set for the analyses of the primary efficacy analysis.

Complete Response (pCR) Rate

The primary efficacy objective for this study is to evaluate the efficacy of Atezo+Tira or Atezo following nCRT on the pathological complete response (pCR) rate, separately.

The pCR rate is defined as the proportion of patients achieving pCR in surgery-resected sample(s) evaluated by the local pathologist at each site.

The number and percentage of the patients with pCR (pCR rate) for each arm is provided with corresponding 95% Clopper-Pearson confidence intervals. If the lower bound of 95% CI is greater than 15%, the pCR rate of the preoperative chemoradiotherapy (based on the historical data), the superiority of Atezo+Tira (or Atezo) is claimed.

pCR rate is additionally assessed in an mITT analysis set. Since this is an exploratory study, no multiplicity adjustment is performed.

### Secondary Efficacy Endpoints

No comparisons between the two arms are performed in secondary endpoint analyses.

### R0 Resection Rate

R0 resection rate is defined as the proportion of patients with a microscopically margin-negative resection, in which no gross or microscopic tumor remains in the primary tumor bed and/or sampled regional lymph nodes based on evaluation by the pathologist.

R0 resection rate of each arm is evaluated based on the mITT population with corresponding 95% Clopper-Pearson confidence intervals provided.

### Objective Response Rate (ORR) Before Surgery

ORR before surgery, defined as the proportion of patients with a complete response (CR) or partial response (PR), as determined by the investigator according to RECIST v1.1.

Objective response rate (ORR) of each arm, as assessed by the investigator according to RECIST v1.1, is estimated using binomial distributions and analyzed in frequency tables with corresponding 95% Clopper-Pearson confidence intervals.

### Relapse-Free Survival (RFS) Rate

The one/two/three-year RFS rate is defined as the proportion of patients who have not experienced disease relapse or death from any cause at 1/2/3 year(s), as determined by the investigator.

Patients who have not experienced disease relapse or death are censored at the time of the last tumor or disease recurrence assessment. Patients with no post-baseline tumor or disease recurrence assessment are censored at the date of randomization.

The RFS analysis is performed on the mITT population. RFS rates at 1, 2, and 3 years of each arm are obtained by using the Kaplan-Meier (KM) Approach together with associated 95% confidence intervals.

### Event Free Survival (1/2/3-EFS) Rate

One/two/three-year event free survival (1/2/3-EFS) rate, defined as the proportion of patients who have not experienced the following events at 1/2/3 year(s) from randomization: progression of disease that precludes surgery, local or distant recurrence, or death due to any cause.

The censoring rule and analysis of EFS rate is conducted in the same manner as the RFS rate.

### Subgroup Analysis

Subgroup analysis is performed on the primary endpoint of pCR and the secondary endpoints of DFS/EFS. The analysis population(s) are the same as those in the primary or secondary endpoint analyses.

Subgroup analysis is conducted with the subgroup population by following biomarkers with PD-L1/PVR/TIGIT positive expression in baseline, pre-surgery, and surgery tumor tissue. The descriptive statistics of the above biomarkers is provided.

### Safety Analyses

The safety analyses are performed on the safety analysis set and separated by different periods:
nCRT period: the period after randomization and before first dose of study immunotherapy (or 30 days after last exposure to chemoradiotherapy if there is no study immunotherapy received).
Sequential immunotherapy period: from first dose of study immunotherapy to surgery (or 30 days after last exposure to study immunotherapy if there is no surgery received).
Post-surgery period: the period after surgery.

Safety is assessed through summaries of exposure to study treatment, adverse events, changes in laboratory test results, and changes in vital signs and ECGs.

Study treatment exposure (such as treatment duration, total dose received, and number of cycles and dose modifications) is summarized with descriptive statistics.

All verbatim adverse event terms are mapped to Medical Dictionary for Regulatory Activities (MedDRA) thesaurus terms, and adverse event severity is graded according to NCI CTCAE v5.0, and severity for CRS is also graded by the investigator according to the ASTCT consensus grading scale. All adverse events, serious adverse events, adverse events leading to death, adverse events of special interest, and adverse events leading to study treatment discontinuation that occur on or after the first dose of study treatment (i.e., treatment-emergent adverse events) is summarized by mapped term, appropriate thesaurus level, and severity grade. For events of varying severity, the highest grade is used in the summaries. Deaths and cause of death are summarized.

Relevant laboratory, vital sign (respiratory rate, pulse rate, systolic and diastolic blood pressure, and temperature), and ECG data are displayed by time, with grades identified where appropriate. Additionally, a shift table of selected laboratory tests is used to summarize the baseline and maximum post-baseline severity grade. Changes in vital signs and ECGs are summarized.

### Exploratory Biomarker Analyses

The exploratory biomarker endpoints that are planned include TILs in baseline, pre-surgery, and surgery tissue samples, and biomarkers in ctDNA extracted from peripheral blood samples.

Descriptive statistics are provided for each exploratory biomarker endpoint. Subgroup analysis based on these exploratory endpoints is conducted if applicable.

### Interim Analyses

An IMC review evaluates safety data until 30 days after the first 20 randomized patients' completing surgery or lost-follow-up.

The final analysis of pCR rate is performed 6 months after the last patient is enrolled in the study. At the same time, one-year RFS rate is analyzed. Two-year and three-year RFS rate is performed at 18 months and 30 months after the last patient's enrollment, respectively.

### Other Embodiments

Although the foregoing invention has been described in some detail by way of illustration and example for purposes of clarity of understanding, the descriptions and examples should not be construed as limiting the scope of the invention.

What is claimed is:

1. A method for treating a subject having a gastric carcinoma (GC) or a gastroesophageal junction carcinoma (GEJC), the method comprising administering to the subject one or more dosing cycles of an anti-TIGIT antagonist antibody, a PD-1 axis binding antagonist, capecitabine, and oxaliplatin.

2. The method of claim 1, wherein the GC or GEJC is an inoperable, locally advanced, metastatic, or advanced GC or GEJC.

3. The method of claim 1 or 2, wherein the GC or GEJC is HER2-negative.

4. The method of any one of claims 1-3, wherein the GC or GEJC is an adenocarcinoma.

5. The method of any one of claims 1-4, wherein the subject has not received a prior systemic therapy for GC or GEJC.

6. The method of any one of claims 1-5, wherein the method comprises administering to the subject:
(a) the anti-TIGIT antagonist antibody at a fixed dose of about 600 mg every three weeks;
(b) the PD-1 axis binding antagonist at a fixed dose of about 1200 mg every three weeks;
(c) capecitabine at a dose of 1000 mg/m² twice daily for two weeks; and
(d) oxaliplatin at a dose of 130 mg/m² every three weeks.

7. The method of any one of claims 1-6, wherein the length of each of the one or more dosing cycles is 21 days.

8. The method of claim 7, wherein the method comprises administering to the subject the anti-TIGIT antagonist antibody, the PD-1 axis binding antagonist, and oxaliplatin on about Day 1 of each of the one or more dosing cycles.

9. The method of claim 7 or 8, wherein the method comprises administering to the subject capecitabine on Days 1-14 of each of the one or more dosing cycles.

10. The method of any one of claims 1-9, wherein the method comprises administering to the subject the PD-1 axis binding antagonist before the anti-TIGIT antagonist antibody.

11. The method of any one of claims 1-10, wherein the method comprises administering to the subject the anti-TIGIT antagonist antibody, the PD-1 axis binding antagonist, and the oxaliplatin intravenously.

12. The method of any one of claims 1-11, wherein the method comprises administering to the subject the capecitabine orally.

13. The method of any one of claims 1-12, wherein the treating results in an increase in objective response rate (ORR) as compared to a reference ORR.

14. The method of claim 13, wherein the reference ORR is an ORR of a population of subjects who have received:
(a) a treatment comprising capecitabine and oxaliplatin and not comprising a PD-1 axis binding antagonist and an anti-TIGIT antagonist antibody; and/or
(b) a treatment comprising capecitabine, oxaliplatin, and a PD-1 axis binding antagonist and not comprising an anti-TIGIT antagonist antibody.

15. The method of any one of claims 1-14, wherein the treating results in an increase in progression-free survival (PFS) as compared to a reference PFS.

16. The method of claim 15, wherein the reference PFS is a median PFS of a population of subjects who have received:
(a) a treatment comprising capecitabine and oxaliplatin and not comprising a PD-1 axis binding antagonist and an anti-TIGIT antagonist antibody; and/or
(b) a treatment comprising capecitabine, oxaliplatin, and a PD-1 axis binding antagonist and not comprising an anti-TIGIT antagonist antibody.

17. The method of any one of claims 1-16, wherein the treating results in an increase in overall survival (OS) as compared to a reference OS.

18. The method of claim 17, wherein the reference OS is a median OS of a population of subjects who have received:
(a) a treatment comprising capecitabine and oxaliplatin and not comprising a PD-1 axis binding antagonist and an anti-TIGIT antagonist antibody; and/or
(b) a treatment comprising capecitabine, oxaliplatin, and a PD-1 axis binding antagonist and not comprising an anti-TIGIT antagonist antibody.

19. The method of any one of claims 1-18, wherein the treating results in an increase in duration of response (DOR) as compared to a reference DOR.

20. The method of claim 19, wherein the reference DOR is a median DOR of a population of subjects who have received:
(a) a treatment comprising capecitabine and oxaliplatin and not comprising a PD-1 axis binding antagonist and an anti-TIGIT antagonist antibody; and/or
(b) a treatment comprising capecitabine, oxaliplatin, and a PD-1 axis binding antagonist and not comprising an anti-TIGIT antagonist antibody.

21. A method for treating a subject having a rectal cancer, the method comprising administering to the subject one or more dosing cycles of an anti-TIGIT antagonist antibody and a PD-1 axis binding antagonist, wherein the one or more dosing cycles are performed following a neoadjuvant chemotherapy (nCRT) regimen.

22. The method of claim 21, wherein the rectal cancer is a locally advanced rectal cancer (LARC).

23. The method of claim 21 or 22, wherein the rectal cancer is a stage cT₃N+M₀ or stage cT₄N_{any}M₀ rectal cancer.

24. The method of any one of claims 21-23, wherein the rectal cancer is an adenocarcinoma.

25. The method of any one of claims 21-24, wherein the subject does not have synchronous colon cancer.

26. The method of any one of claims 21-25, wherein the subject has not received a prior therapy for rectal cancer.

27. The method of any one of claims 21-26, wherein the method comprises administering to the subject the anti-TIGIT antagonist antibody at a fixed dose of about 600 mg every three weeks and the PD-1 axis binding antagonist at a fixed dose of about 1200 mg every three weeks.

28. The method of any one of claims 21-27, wherein the length of each of the one or more dosing cycles is 21 days.

29. The method of any one of claims 21-28, wherein the method comprises administering to the subject the anti-TIGIT antagonist antibody and the PD-1 axis binding antagonist on about Day 1 of each of the one or more dosing cycles.

30. The method of any one of claims 21-29, wherein the method comprises administering to the subject the PD-1 axis binding antagonist before the anti-TIGIT antagonist antibody.

31. The method of any one of claims 21-30, wherein the method comprises administering to the subject the anti-TIGIT antagonist antibody and PD-1 axis binding antagonist intravenously.

32. The method of any one of claims 21-31, wherein the one or more dosing cycles are initiated about two weeks after the last cycle of nCRT.

33. The method of any one of claims 21-32, wherein the one or more dosing cycles are initiated within four weeks after the last cycle of nCRT.

34. The method of any one of claims 21-33, wherein the nCRT regimen comprises radiotherapy delivered to the pelvis at a fraction of about 1.8 Gy per treatment.

35. The method of claim 34, wherein the radiotherapy is administered on Days 1-5 every week.

36. The method of any one of claims 21-35, wherein the nCRT regimen comprises administering a total of between about 45 and about 50.4 Gy of the radiotherapy to the subject.

37. The method of any one of claims 21-36, wherein the radiotherapy is administered in 25 to 28 fractions.

38. The method of any one of claims 21-37, wherein the nCRT regimen comprises a fluoropyrimidine-based chemotherapy.

39. The method of claim 38, wherein the fluoropyrimidine-based chemotherapy is capecitabine or 5-fluorouracil (5-FU).

40. The method of claim 39, wherein the capecitabine is administered orally at a dose of about 825 mg/m².

41. The method of claim 39 or 40, wherein the capecitabine is administered orally twice daily on five consecutive days every week.

42. The method of claim 39 or 40, wherein the capecitabine is administered orally twice daily on seven consecutive days every week.

43. The method of claim 39, wherein the 5-FU is administered intravenously at a dose of about 225 mg/m².

44. The method of claim 39 or 42, wherein the 5-FU is administered on five consecutive days every week.

45. The method of claim 39 or 42, wherein the 5-FU is administered on seven consecutive days every week.

46. The method of any one of claims 21-45, wherein the nCRT is performed for 5 cycles.

47. The method of any one of claims 21-46, wherein the first dosing cycle of the anti-TIGIT antagonist antibody and PD-1 axis binding antagonist is initiated prior to a surgery.

48. The method of claim 47, wherein three dosing cycles are completed prior to the surgery.

49. The method of claim 47 or 48, wherein the surgery is performed within about four weeks after the last dosing cycle.

50. The method of any one of claims 47-49, wherein the surgery is radical surgical resection using total mesorectal excision (TME) and lymph node dissection.

51. The method of any one of claims 21-50, wherein the treating results in a pathological complete response (pCR) and/or an increase in pCR rate as compared to a reference pCR rate.

52. The method of claim 51, wherein the reference pCR rate is a pCR rate of population of subjects who have received a treatment comprising:
(a) nCRT not followed by treatment with a PD-1 axis binding antagonist and an anti-TIGIT antagonist antibody; and/or
(b) nCRT followed by treatment with a PD-1 axis binding antagonist.

53. The method of any one of claims 21-52, wherein the treating results in an increase in R0 resection rate as compared to a reference R0 resection rate.

54. The method of claim 53, wherein the reference R0 resection rate is an R0 resection rate of a population of subjects who have received a treatment comprising:
(a) nCRT not followed by treatment with a PD-1 axis binding antagonist and an anti-TIGIT antagonist antibody; and/or
(b) nCRT followed by treatment with a PD-1 axis binding antagonist.

55. The method of any one of claims 21-54, wherein the treating results in an increase in objective response rate (ORR) as compared to a reference ORR.

56. The method of claim 55, wherein the reference ORR is an ORR of a population of subjects who have received a treatment comprising:
(a) nCRT not followed by treatment with a PD-1 axis binding antagonist and an anti-TIGIT antagonist antibody; and/or
(b) nCRT followed by treatment with a PD-1 axis binding antagonist.

57. The method of any one of claims 21-56, wherein the treating results in an increase in relapse-free survival (RFS) rate as compared to a reference RFS rate.

58. The method of claim 57, wherein the reference RFS rate is an RFS rate of a population of subjects who have received a treatment comprising:
(a) nCRT not followed by treatment with a PD-1 axis binding antagonist and an anti-TIGIT antagonist antibody; and/or
(b) nCRT followed by treatment with a PD-1 axis binding antagonist.

59. The method of claim 57 or 58, wherein the RFS rate is a one-year RFS rate, a two-year RFS rate, or a three-year RFS rate.

60. The method of any one of claims 21-59, wherein the treating results in an increase in event-free survival (EFS) rate as compared to a reference EFS rate.

61. The method of claim 60, wherein the reference EFS rate is an EFS rate of a population of subjects who have received a treatment comprising:
(a) nCRT not followed by treatment with a PD-1 axis binding antagonist and an anti-TIGIT antagonist antibody; and/or
(b) nCRT followed by treatment with a PD-1 axis binding antagonist.

62. The method of claim 60 or 61, wherein the EFS rate is a one-year RFS rate, a two-year EFS rate, or a three-year EFS rate.

63. The method of any one of claims 1-62, wherein the anti-TIGIT antagonist antibody comprises the following hypervariable regions (HVRs):
an HVR-H1 sequence comprising the amino acid sequence of SNSAAWN (SEQ ID NO: 11);
an HVR-H2 sequence comprising the amino acid sequence of KTYYRFKWYSDYAVSVKG (SEQ ID NO: 12);
an HVR-H3 sequence comprising the amino acid sequence of ESTTYDLLAGPFDY (SEQ ID NO: 13);
an HVR-L1 sequence comprising the amino acid sequence of KSSQTVLYSSNNKKYLA (SEQ ID NO: 14);
an HVR-L2 sequence comprising the amino acid sequence of WASTRES (SEQ ID NO: 15); and
an HVR-L3 sequence comprising the amino acid sequence of QQYYSTPFT (SEQ ID NO: 16).

64. The method of claim 63, wherein the anti-TIGIT antagonist antibody further comprises the following light chain variable region framework regions (FRs):
an FR-L1 comprising the amino acid sequence of DIVMTQSPDSLAVSLGERATINC (SEQ ID NO: 17);
an FR-L2 comprising the amino acid sequence of WYQQKPGQPPNLLIY (SEQ ID NO: 18);
an FR-L3 comprising the amino acid sequence of GVPDRFSGSGSGTDFTLTISSLQAEDVAVYYC (SEQ ID NO: 19); and
an FR-L4 comprising the amino acid sequence of FGPGTKVEIK (SEQ ID NO: 20).

65. The method of claim 63, wherein the anti-TIGIT antagonist antibody further comprises the following heavy chain variable region FRs:
an FR-H1 comprising the amino acid sequence of X₁VQLQQSGPGLVKPSQTLSLTCAISGDSVS (SEQ ID NO: 21), wherein X₁ is E or Q;
an FR-H2 comprising the amino acid sequence of WIRQSPSRGLEWLG (SEQ ID NO: 22);
an FR-H3 comprising the amino acid sequence of RITINPDTSKNQFSLQLNSVTPEDTAVFYCTR (SEQ ID NO: 23); and
an FR-H4 comprising the amino acid sequence of WGQGTLVTVSS (SEQ ID NO: 24).

66. The method of claim 65, wherein X₁ is E.

67. The method of claim 65, wherein X₁ is Q.

68. The method of any one of claims 1-67, wherein the anti-TIGIT antagonist antibody comprises:
(a) a heavy chain variable (VH) domain comprising an amino acid sequence having at least 95% sequence identity to the amino acid sequence of SEQ ID NO: 27 or 28;
(b) a light chain variable (VL) domain comprising an amino acid sequence having at least 95% sequence identity to the amino acid sequence of SEQ ID NO: 29; or
(c) a VH domain as in (a) and a VL domain as in (b).

69. The method of any one of claims 1-68, wherein the anti-TIGIT antagonist antibody comprises:
(a) a VH domain comprising the amino acid sequence of SEQ ID NO: 27 and a VL domain comprising the amino acid sequence of SEQ ID NO: 29; or
(b) a VH domain comprising the amino acid sequence of SEQ ID NO: 28 and a VL domain comprising the amino acid sequence of SEQ ID NO: 29.

70. The method of any one of claims 1-69, wherein the anti-TIGIT antagonist antibody is a monoclonal antibody.

71. The method of claim 70, wherein the anti-TIGIT antagonist antibody is a human antibody.

72. The method of any one of claims 1-71, wherein the anti-TIGIT antagonist antibody is a full-length antibody.

73. The method of any one of claims 1-66 and 68-72, wherein the anti-TIGIT antagonist antibody is tiragolumab.

74. The method of any one of claims 1-71, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

75. The method of any one of claims 1-74, wherein the anti-TIGIT antagonist antibody is an IgG class antibody.

76. The method of claim 75, wherein the IgG class antibody is an IgG1 subclass antibody.

77. The method of any one of claims 1-62, wherein the anti-TIGIT antagonist antibody is tiragolumab, vibostolimab, etigilimab, EOS084448, SGN-TGT, TJ-T6, BGB-A1217, AB308, domvanalimab, BMS-986207, ASP8374, or COM902.

78. The method of any one of claims 1-77, wherein the method comprises administering to the subject the PD-1 axis binding antagonist at a fixed dose of about 1200 mg every three weeks.

79. The method of any one of claims 1-78, wherein the PD-1 axis binding antagonist is selected from the group consisting of a PD-L1 binding antagonist, a PD-1 binding antagonist, and a PD-L2 binding antagonist.

80. The method of claim 79, wherein the PD-1 axis binding antagonist is a PD-L1 binding antagonist.

81. The method of claim 80, wherein the PD-L1 binding antagonist inhibits the binding of PD-L1 to one or more of its ligand binding partners.

82. The method of claim 81, wherein the PD-L1 binding antagonist inhibits the binding of PD-L1 to PD-1, B7-1, or both PD-1 and B7-1.

83. The method of any one of claims 79-82, wherein the PD-L1 binding antagonist is an anti-PD-L1 antagonist antibody.

84. The method of claim 83, wherein the anti-PD-L1 antagonist antibody is atezolizumab, MDX-1105, durvalumab, avelumab, SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636.

85. The method of claim 84, wherein the anti-PD-L1 antagonist antibody is atezolizumab.

86. The method of any one of claims 1-85, wherein the anti-PD-L1 antagonist antibody comprises the following HVRs:
an HVR-H1 sequence comprising the amino acid sequence of GFTFSDSWIH (SEQ ID NO: 3);
an HVR-H2 sequence comprising the amino acid sequence of AWISPYGGSTYYADSVKG (SEQ ID NO: 4);
an HVR-H3 sequence comprising the amino acid sequence of RHWPGGFDY (SEQ ID NO: 5);
an HVR-L1 sequence comprising the amino acid sequence of RASQDVSTAVA (SEQ ID NO: 6);
an HVR-L2 sequence comprising the amino acid sequence of SASFLYS (SEQ ID NO: 7); and
an HVR-L3 sequence comprising the amino acid sequence of QQYLYHPAT (SEQ ID NO: 8).

87. The method of any one of claims 1-86, wherein the anti-PD-L1 antagonist antibody comprises:
(a) a heavy chain variable (VH) domain comprising an amino acid sequence having at least 95% sequence identity to the amino acid sequence of SEQ ID NO: 9;
(b) a light chain variable (VL) domain comprising an amino acid sequence having at least 95% sequence identity to the amino acid sequence of SEQ ID NO: 10; or
(c) a VH domain as in (a) and a VL domain as in (b).

88. The method of any one of claims 1-87, wherein the anti-PD-L1 antagonist antibody comprises:
(a) a VH domain comprising the amino acid sequence of SEQ ID NO: 9; and
(b) a VL domain comprising the amino acid sequence of SEQ ID NO: 10.

89. The method of any one of claims 86-88, wherein the anti-PD-L1 antagonist antibody is a monoclonal antibody.

90. The method of claim 89, wherein the anti-PD-L1 antagonist antibody is a humanized antibody.

91. The method of claim 89 or 90, wherein the anti-PD-L1 antagonist antibody is a full-length antibody.

92. The method of any one of claims 89-91, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

93. The method of any one of claims 89-92, wherein the anti-PD-L1 antagonist antibody is an IgG class antibody.

94. The method of claim 93, wherein the IgG class antibody is an IgG1 subclass antibody.

95. The method of claim 79, wherein the PD-1 axis binding antagonist is a PD-1 binding antagonist.

96. The method of claim 95, wherein the PD-1 binding antagonist inhibits the binding of PD-1 to one or more of its ligand binding partners.

97. The method of claim 96, wherein the PD-1 binding antagonist inhibits the binding of PD-1 to PD-L1, PD-L2, or both PD-L1 and PD-L2.

98. The method of any one of claims 79 and 95-97, wherein the PD-1 binding antagonist is an anti-PD-1 antagonist antibody.

99. The method of claim 98, wherein the anti-PD-1 antagonist antibody is nivolumab, pembrolizumab, MEDI-0680, spartalizumab, cemiplimab, BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, or hAb21.

100. The method of any one of claims 79 and 95-97, wherein the PD-1 binding antagonist is an Fc fusion protein.

101. The method of claim 100, wherein the Fc fusion protein is AMP-224.

102. The method of any one of claims 1-101, wherein the subject is a human.


SEQUENCE LISTING (34 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 11 - protein, 7 aa, synthetic construct
SNSAAWN

SEQ ID NO: 12 - protein, 18 aa, synthetic construct
KTYYRFKWYSDYAVSVKG

SEQ ID NO: 13 - protein, 14 aa, synthetic construct
ESTTYDLLAGPFDY

SEQ ID NO: 14 - protein, 17 aa, synthetic construct
KSSQTVLYSSNNKKYLA

SEQ ID NO: 15 - protein, 7 aa, synthetic construct
WASTRES

SEQ ID NO: 16 - protein, 9 aa, synthetic construct
QQYYSTPFT

SEQ ID NO: 17 - protein, 23 aa, synthetic construct
DIVMTQSPDSLAVSLGERATINC

SEQ ID NO: 18 - protein, 15 aa, synthetic construct
WYQQKPGQPPNLLIY

SEQ ID NO: 19 - protein, 32 aa, synthetic construct
GVPDRFSGSGSGTDFTLTISSLQAEDVAVYYC

SEQ ID NO: 20 - protein, 10 aa, synthetic construct
FGPGTKVEIK

SEQ ID NO: 21 - protein, 30 aa, synthetic construct
  SITE 1: note = misc_feature - Xaa is Gln or Glu
XVQLQQSGPGLVKPSQTLSLTCAISGDSVS

SEQ ID NO: 22 - protein, 14 aa, synthetic construct
WIRQSPSRGLEWLG

SEQ ID NO: 23 - protein, 32 aa, synthetic construct
RITINPDTSKNQFSLQLNSVTPEDTAVFYCTR

SEQ ID NO: 24 - protein, 11 aa, synthetic construct
WGQGTLVTVSS

SEQ ID NO: 25 - protein, 30 aa, synthetic construct
EVQLQQSGPGLVKPSQTLSLTCAISGDSVS

SEQ ID NO: 26 - protein, 30 aa, synthetic construct
QVQLQQSGPGLVKPSQTLSLTCAISGDSVS

SEQ ID NO: 27 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 28 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 29 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC